SEQUENCE LISTING (63 sequences)

SEQ ID NO: 1 - DNA, 278 bp, synthetic construct
  source 1..278: mol_type = other DNA
ccggtagggctctgcgtttgctccaggtagtccgctgctcccttgggcctgggcccactgacagccctggtgcctctggccggctgcacacctcctggcgggcagctgtgaaaacttcttcacattcagcctgttctggcaatacctgggctgaatcaggagaagttttcacggaggcctgccctgactgcccacggtgccgtggccaaagaggatctaagggcaccgctgagggcctacctaaccatcgtggggaataaggacagtgtcaccggatc

SEQ ID NO: 2 - DNA, 279 bp, synthetic construct
  source 1..279: mol_type = other DNA
ccggtagggctctgcgtttgctccaggtagtccgctgctcccttgggcctgggcccactgacagccctggtgcctctggccggctgcacacctcctggcgggcagctgtgtaaggattcataaagcttccttgttctggcaatacctgaggaagctaaacgaatccttacacggaggcctgccctgactgcccacggtgccgtggccaaagaggatctaagggcaccgctgagggcctacctaaccatcgtggggaataaggacagtgtcacccggatc

SEQ ID NO: 3 - DNA, 279 bp, synthetic construct
  source 1..279: mol_type = other DNA
ccggtagggctctgcgtttgctccaggtagtccgctgctcccttgggcctgggcccactgacagccctggtgcctctggccggctgcacacctcctggcgggcagctgtgtcatcatagtgaagtcttccttgttctggcaatacctgaggaagacaacgctatgatgacacggaggcctgccctgactgcccacggtgccgtggccaaagaggatctaagggcaccgctgagggcctacctaaccatcgtggggaataaggacagtgtcacccggatc

SEQ ID NO: 4 - DNA, 279 bp, synthetic construct
  source 1..279: mol_type = other DNA
ccggtagggctctgcgtttgctccaggtagtccgctgctcccttgggcctgggcccactgacagccctggtgcctctggccggctgcacacctcctggcgggcagctgtgaaggattcataaagcttcctctgttctggcaatacctggaggaagcaatgtgaatccttcacggaggcctgccctgactgcccacggtgccgtggccaaagaggatctaagggcaccgctgagggcctacctaaccatcgtggggaataaggacagtgtcacccggatc

SEQ ID NO: 5 - DNA, 279 bp, synthetic construct
  source 1..279: mol_type = other DNA
ccggtagggctctgcgtttgctccaggtagtccgctgctcccttgggcctgggcccactgacagccctggtgcctctggccggctgcacacctcctggcgggcagctgtgagtgtcgtgacagcgagggcgtgttctggcaatacctgcgccctcggagccacgacactcacggaggcctgccctgactgcccacggtgccgtggccaaagaggatctaagggcaccgctgagggcctacctaaccatcgtggggaataaggacagtgtcacccggatc

SEQ ID NO: 6 - DNA, 3642 bp, synthetic construct
  source 1..3642: mol_type = other DNA
ctgcgcgctcgctcgctcactgaggccgccggcccaaagggccggcgtcgggcgacctttggtcgcccggcctcagtgagcgagcgagcgcgcagagagggagtggagatctgaattcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtagggctctgcgtttgctccaggtagtccgctgctcccttgggcctgggcccactgacagccctggtgcctctggccggctgcacacctcctggcgggcagctgtgaaaacttcttcacattcagcctgttctggcaatacctgggctgaatcaggagaagttttcacggaggcctgccctgactgcccacggtgccgtggccaaagaggatctaagggcaccgctgagggcctacctaaccatcgtggggaataaggacagtgtcaccggatccctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaacagcaggcatgctggggatgcggtgggctctatggcttctgaggcggaaagaaccagctttggacgcgtgcatgcagatctaggaacccctagtgatggagttggccactccctctctgcgcgctcgctcgctcactgaggccgggcgaccaaaggtcgcccgacgccggccctttgggccggcggcctcagtgagcgagcgagcgcgcagagagggagtggccaagatggagtcagctgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcagggtttaaaccgcggaacccctatttgtttatttttctaaatacattcaaatatgtatccgctcatgagacaataaccctgataaatgcttcaataatattgaaaaaggaagagtatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgtgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagtttaaacctgtcagaccaagtttactcatatatactttagattgatttaaaacttcatttttaatttaaaaggatctaggtacatgtgaagatcctttttgataatctcatgaccaaaatcccttaacgtgagttttcgttccactgagcgtcagaccccggcggccgcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaagcggccgcaacgccagcaacgcggcctttttacggttcctggccttttgctggccttttgctcacgtgttctttcctgcgttatcccctgattctgtggataaccgtattaccgcctttgagtgagctgataccgctcgccgcagccgaacgagacagctgaaacgtta

SEQ ID NO: 7 - DNA, 3643 bp, synthetic construct
  source 1..3643: mol_type = other DNA
ctgcgcgctcgctcgctcactgaggccgccggcccaaagggccggcgtcgggcgacctttggtcgcccggcctcagtgagcgagcgagcgcgcagagagggagtggagatctgaattcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtagggctctgcgtttgctccaggtagtccgctgctcccttgggcctgggcccactgacagccctggtgcctctggccggctgcacacctcctggcgggcagctgtgtaaggattcataaagcttccttgttctggcaatacctgaggaagctaaacgaatccttacacggaggcctgccctgactgcccacggtgccgtggccaaagaggatctaagggcaccgctgagggcctacctaaccatcgtggggaataaggacagtgtcacccggatccctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaacagcaggcatgctggggatgcggtgggctctatggcttctgaggcggaaagaaccagctttggacgcgtgcatgcagatctaggaacccctagtgatggagttggccactccctctctgcgcgctcgctcgctcactgaggccgggcgaccaaaggtcgcccgacgccggccctttgggccggcggcctcagtgagcgagcgagcgcgcagagagggagtggccaagatggagtcagctgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcagggtttaaaccgcggaacccctatttgtttatttttctaaatacattcaaatatgtatccgctcatgagacaataaccctgataaatgcttcaataatattgaaaaaggaagagtatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgtgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagtttaaacctgtcagaccaagtttactcatatatactttagattgatttaaaacttcatttttaatttaaaaggatctaggtacatgtgaagatcctttttgataatctcatgaccaaaatcccttaacgtgagttttcgttccactgagcgtcagaccccggcggccgcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaagcggccgcaacgccagcaacgcggcctttttacggttcctggccttttgctggccttttgctcacgtgttctttcctgcgttatcccctgattctgtggataaccgtattaccgcctttgagtgagctgataccgctcgccgcagccgaacgagacagctgaaacgtta

SEQ ID NO: 8 - DNA, 3643 bp, synthetic construct
  source 1..3643: mol_type = other DNA
ctgcgcgctcgctcgctcactgaggccgccggcccaaagggccggcgtcgggcgacctttggtcgcccggcctcagtgagcgagcgagcgcgcagagagggagtggagatctgaattcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtagggctctgcgtttgctccaggtagtccgctgctcccttgggcctgggcccactgacagccctggtgcctctggccggctgcacacctcctggcgggcagctgtgtcatcatagtgaagtcttccttgttctggcaatacctgaggaagacaacgctatgatgacacggaggcctgccctgactgcccacggtgccgtggccaaagaggatctaagggcaccgctgagggcctacctaaccatcgtggggaataaggacagtgtcacccggatccctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaacagcaggcatgctggggatgcggtgggctctatggcttctgaggcggaaagaaccagctttggacgcgtgcatgcagatctaggaacccctagtgatggagttggccactccctctctgcgcgctcgctcgctcactgaggccgggcgaccaaaggtcgcccgacgccggccctttgggccggcggcctcagtgagcgagcgagcgcgcagagagggagtggccaagatggagtcagctgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcagggtttaaaccgcggaacccctatttgtttatttttctaaatacattcaaatatgtatccgctcatgagacaataaccctgataaatgcttcaataatattgaaaaaggaagagtatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgtgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagtttaaacctgtcagaccaagtttactcatatatactttagattgatttaaaacttcatttttaatttaaaaggatctaggtacatgtgaagatcctttttgataatctcatgaccaaaatcccttaacgtgagttttcgttccactgagcgtcagaccccggcggccgcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaagcggccgcaacgccagcaacgcggcctttttacggttcctggccttttgctggccttttgctcacgtgttctttcctgcgttatcccctgattctgtggataaccgtattaccgcctttgagtgagctgataccgctcgccgcagccgaacgagacagctgaaacgtta

SEQ ID NO: 9 - DNA, 3643 bp, synthetic construct
  source 1..3643: mol_type = other DNA
ctgcgcgctcgctcgctcactgaggccgccggcccaaagggccggcgtcgggcgacctttggtcgcccggcctcagtgagcgagcgagcgcgcagagagggagtggagatctgaattcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtagggctctgcgtttgctccaggtagtccgctgctcccttgggcctgggcccactgacagccctggtgcctctggccggctgcacacctcctggcgggcagctgtgaaggattcataaagcttcctctgttctggcaatacctggaggaagcaatgtgaatccttcacggaggcctgccctgactgcccacggtgccgtggccaaagaggatctaagggcaccgctgagggcctacctaaccatcgtggggaataaggacagtgtcacccggatccctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaacagcaggcatgctggggatgcggtgggctctatggcttctgaggcggaaagaaccagctttggacgcgtgcatgcagatctaggaacccctagtgatggagttggccactccctctctgcgcgctcgctcgctcactgaggccgggcgaccaaaggtcgcccgacgccggccctttgggccggcggcctcagtgagcgagcgagcgcgcagagagggagtggccaagatggagtcagctgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcagggtttaaaccgcggaacccctatttgtttatttttctaaatacattcaaatatgtatccgctcatgagacaataaccctgataaatgcttcaataatattgaaaaaggaagagtatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgtgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagtttaaacctgtcagaccaagtttactcatatatactttagattgatttaaaacttcatttttaatttaaaaggatctaggtacatgtgaagatcctttttgataatctcatgaccaaaatcccttaacgtgagttttcgttccactgagcgtcagaccccggcggccgcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaagcggccgcaacgccagcaacgcggcctttttacggttcctggccttttgctggccttttgctcacgtgttctttcctgcgttatcccctgattctgtggataaccgtattaccgcctttgagtgagctgataccgctcgccgcagccgaacgagacagctgaaacgtta

SEQ ID NO: 10 - DNA, 3643 bp, synthetic construct
  source 1..3643: mol_type = other DNA
ctgcgcgctcgctcgctcactgaggccgccggcccaaagggccggcgtcgggcgacctttggtcgcccggcctcagtgagcgagcgagcgcgcagagagggagtggagatctgaattcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtagggctctgcgtttgctccaggtagtccgctgctcccttgggcctgggcccactgacagccctggtgcctctggccggctgcacacctcctggcgggcagctgtgagtgtcgtgacagcgagggcgtgttctggcaatacctgcgccctcggagccacgacactcacggaggcctgccctgactgcccacggtgccgtggccaaagaggatctaagggcaccgctgagggcctacctaaccatcgtggggaataaggacagtgtcacccggatccctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaacagcaggcatgctggggatgcggtgggctctatggcttctgaggcggaaagaaccagctttggacgcgtgcatgcagatctaggaacccctagtgatggagttggccactccctctctgcgcgctcgctcgctcactgaggccgggcgaccaaaggtcgcccgacgccggccctttgggccggcggcctcagtgagcgagcgagcgcgcagagagggagtggccaagatggagtcagctgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcagggtttaaaccgcggaacccctatttgtttatttttctaaatacattcaaatatgtatccgctcatgagacaataaccctgataaatgcttcaataatattgaaaaaggaagagtatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgtgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagtttaaacctgtcagaccaagtttactcatatatactttagattgatttaaaacttcatttttaatttaaaaggatctaggtacatgtgaagatcctttttgataatctcatgaccaaaatcccttaacgtgagttttcgttccactgagcgtcagaccccggcggccgcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaagcggccgcaacgccagcaacgcggcctttttacggttcctggccttttgctggccttttgctcacgtgttctttcctgcgttatcccctgattctgtggataaccgtattaccgcctttgagtgagctgataccgctcgccgcagccgaacgagacagctgaaacgtta

SEQ ID NO: 11 - DNA, 25180 bp, Homo sapiens
  source 1..25180: mol_type = other DNA
agtgacgctgcggcctccttctgcctaggtcccaacgcttcggggcaggggtgcggtcttgcaataggaagccgagcgtcttgcaagcttcccgtcgggcaccagctactcggccccgcaccctacctggtgcattccctagacacctccggggtccctacctggagatccccggagccccccttcctgcgccagccatgcctttaaaccgcactttgtccatgtcctcactgccaggactggaggactgggaggatgaattcgacctggagaacgcagtgctcttcgaagtggcctgggaggtggctaacaagggtgagcacgccaggcgtcggggcccttgacgagaaggacagtagtggatgggggcccggaactctgagttgtaggaggacggggctactaggggaacccttgtgacctgggcaaggaggctgtgtccttgaggaggggctgggaagccggaggtgggtgggagagggatggttcgtttgacttaagatgaaaggaggctggaagcccgggttctgggtcctagggaagagggcgctaaaatgagactcctaggtcctggaggcaggcttccgagagactagatacctgggtcctgaacagggaaggggatggggggatgggatttctgcgtgcaaacctgggaagctgccggggtttcagacctatgaccctaacagcatggtctaggggataaaactccagatttgagagagagctgactgggagataggaggcctgagcggcggtgggaggacctgaggatctggctgcttaagaataaagatgggacgggctggagactgtaatgaatgcctgggctttgaaggagctgagggcctgagctattagttcctaggagtagaggagcccatggcctgggacacatgcgtactggaataggggaaggctgggaagtcctaagttagtcagaggctgggccctaggatgggaggggctaggagcctgcatttctgtgtcttgggcttcagaaagctgaggttctggccttggagaaggctggggctgaaatccccaggaacaagccaatctcacacctcaaagagcagggtctggggggttgctttgctgggtcttggaggagaaagggactgtgtctcccaggaggagagggggcccgtgaacacccagccgggtatcgccagggcctgggctccaggatgggaggaggctgggggctggggctcctgggtctgggatagggtcatgggagaggcagagccctgcatgctggtgtccctagtcagggagaggaaattgggaggcaggtcagcaaggtccctgggggcagggaatgaccatgcaggcttctcttgggcaatggtgagaagctggattggccatgagggggtctccctcactgggttttggagcctggctgcgatttaaacccagctcctgtgagctgggaactcgggaacctgagtccgggtagtcagtcctcagctctgccatccttagagccccagtgcctaattccttcagaccaaggggtcccggcacccagccccttctctctcagacccagaagtctagcctgagccccctcctccttcagggtcctgaccccagcccccttctcccttagacccaggaggactgccctccagccctcgtgttcccctcatccaagcttccaacccctactccaaactatgggctcccagtcccctcttccccctgagctagaggtctggggcctgagtgccccctacatccccggcccaccccaccccaggccccagaactatggacctcggccggggcctgccctcacacgcttgggcccacagtgggtggcatctacacggtgctgcagacgaaggcgaaggtgacaggggacgaatggggcgacaactacttcctggtggggccgtacacggagcagggcgtgaggacccaggtggaactgctggaggcccccaccccggccctgaagaggacactggattccatgaacagcaagggctgcaaggtgggacgtggcccagcccagggcagaagcgggtggacagcctgccatcggggtgggagccacagacggagagggtgcagggtggacagccccagccagagagcgaggcaggcttggacacaggaaggaggcgtgataggacacccagacagatgcagggcctggaagtgggtgggtggatcccagacatgggcgaatggacagacagatggcaagtatgagacagactgagacggagatccaagaggccaaggtcctcagacccagagagaaagagagcaagatcgaaagacagcctggtcaacatggcgaaaccccgtctctaccaaaaatacaaaaattagctgggtgtggtggcgggtgcctgtaatcccagctactcgggaggctgaggcaggagaatcgcttgaacccgggaggcggaggttgcagtgagctgcgattgggccactgcactccagcctgggcgacggagcgagactctgtctcagaaaagaatgccagccagccaggcagagctggggagaagtgggcctgaggtcaggaaggagcaggattctggagcttgaagttcaagttcaccctccacttgctgcctgtgtgtccttggctaatggacatgctaatagcgtctaccttctagcggtgctgggaggaggaaacagttaataaggacaaagtggttagtagagttcttggcatgaaggtagcacattgtgtgggcggtgattgtcattgttatgatcatcgtcattattgttactaacagccaagcttggagagtgagggggacaggcagaccaagggagaagggtggatggagccacaggcaagagtaatagactggggacagaagagaggccaccagatgtttcagaaagccagtcctagaaggagagagaagcatggaatatcggggaagagaggagaggtcagtggacagacccacaggacagagtctgagatgggcagatcagggattagagaggagggggagttccagggccccagggtgaagacggaaagagacacgctgttgtccaaggagaaagccatcaggcggcacgctgttgtccaaggagaaagccatcaggcggcagggctaagggacagacaggtccaggctggggatggagaggcactgtggttcctttactcattcacagcctctggtttggggtggggtctggaatccattcaggtccgactgtgtccctaagggaacccagaacagcccagtcgcttcctgagggcagctgcttcccacgtgctgggggcctaagaatagcccccccccccccccagcaaaagggcagacaacagctgggagggggagggacctcagagcgaggctccaggcagcaaaaagccaggtcggcgaggagcggggctgcttctccactgctgtggcagcaggcccagaaccaaagccatggtctcagggaggaaaacagccaactggagaaatgtggcagaggcagaagtgagacagcaagtcttagaaacacggcagtggctagaaaaatgcagccctggggcacggacagaggatgggctatgttagaaatgtgtggcgtggccgagggcggtggctcatgcctggaatcccagcactttgggaggccgaggtgagaggatagcttgagcccaggagttcctgacccacctgggcaacatggcgaaactctgtctctactaaaaatacaaaaaattagccaggcatgatggtgcgtgctcgtgatcccagttactcaggaggctgaggtgggaggatgggatgtggaggctgtagtgagccataattttttttttttttttttcagatagaatctcactctgtcgctgagtctggagtgcagtggtacaatctcgactcactgcaacctccgcctcccgcgttcaagcaattctcctgcctcggcctcccaagtagctgggacctcaggcacatgctgccacgcccggctaattttttgtattttagtagagacagggtttcaccatgttgcccaggctggtctcaaactcctgagctcaggcaatccgcccgcctcggccccccaaagagctaggattacaggcgtgagccaccgcgcccagcccagtgagccatgattgcaccactgcactccagcctgggtgaccggagtgagaccctgtcaaaaacaaaaacaaacaaaaaaaaatggaagaaagaaagaaaaaggagagagggagggaaggagggagagagggaaggaaggaaggaaggaagcaaggaaggaaggaaggaggctgtggactacaaaatgggcctatggatagaggaaagaggtcgcaggtgacagatatgtgatgaaaggaacagaaacaaggcatcatggggaagaaaagtggcagcatgaacagtaagagaatcattacaggctgggtgcagtagctcacgcctgtaatcccagcactttggtaggctgaggccaggagtttgagaccagcctgggcaacatggtgaaaccctgtccttacaaaaaagttaaaaattagccgggatgtgataccttgtgcctgtggtcccagctacgtgggaagctgcggtggaaggattgcttgagcctgggagatcgaagcttcagtgaaccgtaattgcaccactcccttccaggctggaggacagagcaagaccccgtctctgaaaataaaaaagggcctgctttaggtggctcacacttctaatctcaacactttgggaggctaagcaagaaaactgcttgaacgcaggagttcacgatcagcctgggcaacatagtgagaccccatctccacaaaaaattaaaaaatcagcctggcatggtggcccacgcctgtaggaggtgaggtgggaggattgactgagccagggaggttgaggctatagtgaaccatgttcacaccactgcactccagcctgggcaacagagcgagaccctgtctgaaaacaaaaaagaagcatcccgtggaaggaattagcatcagaaagaaaagacagtgggagatgcagatggttggtaagagaaatgtggcagcaagtacagaaacaaggcagcaggtaggggaaatgatttgcagagtggccagacagggcagtgggggcatagacacaggacatgtgacatatgtggttataagcagagaaaggaggcagcgagtgggagaagggtagcagttacaggaattaaacaggaggctgggtgcggtggctcacgcctgtaatcccagcactttgggacgccgaggcgggcggatcacaaggtcgggaaatggagatcatcctggctaacacagtgaaacctcgtctctactgaaaatacaaaaaaaagtagccgggcgtggtggcgggccgctgtagtcccagctactcgggaggctgaggcaggagaatggtgtgaacccaggaggcagaggttgcagtgagccgagatcatgccacggcactccagcctgggcaacagagccagactccgtctcaagaaaaaaaaaaaattagccaggtgtgggagcgggtgcctgtaatcccagctacttgggaggctgaggcaggagaattgcttgaacccgggagacagaggttgcagtgagctgagatcgcaccactgcactccagcctgggccacagagtgagactccatctcaaaaaaaagaaagaaagaaagtaagcagggggcacggaaaatattgcaacagagatggaaaaaatgtgtagcaggaataaatcatgcaaatagagaagtaaggctaaggcagcaggtgagaaattagcagcagatatggaaacagaagataagagagctgaggcttaagtctatagacatctgcctgcaaatgggagaaccacggatgagtgaagatatgaggctgaagcccagagcctccctatggtgcctccccaaccccatcccaggtgtatttcgggcgctggctgatcgagggaggccctctggtggtgctcctggacgtgggtgcctcagcttgggccctggagcgctggaagggagagctctgggatacctgcaacatcggagtgccgtggtacgaccgcgaggccaacgacgctgtcctctttggctttctgaccacctggttcctgggtgaggtaggccccgtctgacacattccacccatacctgaaactacaatgaccatcagcccctgggccagaggatgggcttctccctgggcattctgggagtcatagggtcctttaatccaaggattagctctggcctttgaggacatgaacatccaagggcccaggctctactctcgatgccaacttcatttactctagtcttccaagcatagggattcttggaatcagcctcaaagtgaggctggcgggcttgcccgaggaattctaggagttgtagttctctagtgtgatattgctggactcagctctcagatgctgccctggatgctcatgaatccccagcctgacctggttttgacccccttctaggttttaagaggctcaagaagcttagtttccaggctgggcacggtggctcatgcctgtaatcccagcactttgggaggccgatgggggaagatcacgtgaggccaggagttcaagatggccaaacctcgtctttactaaacatacaaaaaaattagctgggtgtggtggtgggcacctgtaatcccaaatcccagctacttgggaggctgagacaggagaattgcttgaacctgggaggcgggaattgcagtgagctgagattgcatcacggcactccagcctgggccacagagtgagacactgtctcaaaaaaaaaaagaaaaagaaaaaaaaaaaaagaagcttaatttctcatgccctgggacaaataattgaggaactggctgctcctatggcattctgggagttgtagtccctttgccaatgttgggtgcagacagcgtacccttgagacaaggggtctttggaaactatgtccaggacctgggaacatgaaaacttcagtctgtccaatctaaccctgccaggacttagatggttggccaaaaaatcctcagttcctgtcaacccctgaggaggcagtcacttgagcccaggagttcaagaccagcctgggctgcctcagggatttacaggtccctgtccggttgggattgctgcagtgggccgctgcccactctgtgcagtagtggtgacagattgacctctccccgagtccttgtccctactggggattgccgattcttttcccaacactcgtggagacagtggccctgtccctgttgcccacagttcctggcacagagtgaggagaagccacatgtggttgctcacttccatgagtggttggcaggcgttggactctgcctgtgtcgtgcccggcgactgcctgtagcaaccatcttcaccacccatgccacgctgctggggcgctacctgtgtgccggtgccgtggacttctacaacaacctggagaacgtgagctgggaccgtggcagatgggggactgcgagtatgccaggaaaacccaaagctctggggggcccaaggggatggcaccccctcttctcccaggatccaggagtccaagtccccagacccctcctacctcagacccaggaatccagcccccagcaccaatttctcttgggaccccagtgtctggacctccagagtcttctccccaatccttctaaccggactgctgtcccctcccacccagttcaacgtggacaaggaagcaggggagaggcagatctaccaccgatactgcatggaaagggcggcagcccactgcgctcacgtcttcactactgtgtcccagatcaccgccatcgaggcacagcacttgctcaagaggaaaccaggtaggggctgggctgaagtccccagacgtgaatgaggtaggcagctgtctatgccaaagccagttgctcctcagcccaagaagcccactgtaagttgaaatagcataaaccgaaaacgtatttcggctgggcatggtggctcacacctgtaatctcagcactttgagaggccaaggcaggagaaccacttgagcccgggaatttgagaccagcctgggcaacatggcgaatccctgtctaaaatattcgccagacatggtgtgtgcctgtatccctagctactcaggaggctgaggtgggaggattgctcgagcctaggaggtcaagggtgcagtgagctgtgatcgtgccattgcatgatttgggtggcctggagagaccctgtcacagcctgggtgacacagtgagaccctgtctcaaaaaataaaaaaatgcatttaatacacctaacctactgaacatcatagcttaggctagtataccttaaatgtgcccagaacactttatgttagcctacgactgggcaaaatcatccaatacaaatctgttttattttatttttatttatttatttatttttgagacggagtctcgctctgtcgcccaggctggagtgcagtggcgctatcttggctcactgtaagctccgcctcccaggttcatgccattctcctgcctcagcctcccaagtagctgggactatacgcgcctgccaccacgcctggctaattttaatacaaacctattttataataaagtgttgaataggctgggcgcagtggctcacacctgtaatcccaacactttgggaggccaaggcgggcagatcacgaggtcaggagatcgagaccagcctggccaacatggcgaaactccatctctactaaaaatacaaaaattagccgggcgtggtggtgggtgcctgtaatcccagctactcgggaggctgaggcaggagaatcacttgaacccaggaggcggaggttgcagtgggccgagatcacaccactgcactccagcctggacaacagagcgagactccatctcaaaaaattaaaaaaaaaaagtgttgaatatctcatcaaaatccaaaattggactcatgtggatgtatgtaactttcacaccatcataaggttgaaaaatcataacttggctgggcacggtggcttacacctgtaatcccagcactttggaaggctgaggtgggcagatcacctgaggtcatgtgctcaagaccagcctggccaacattgtgagaccccctctctgccaaaaatacaaaaattaggtgggcatggtggcaggcgcctgtaatcccagccacccagctactcgggaggctgaggcaggagaatctcttgaacccaggaggcagaggttgtcgtgagccaagattgtgccattgcactccagcctgggcaacaagagcgaaactctgtctcgaaaagaaaaatcataacttggaccatcctaagtcctaagctggggaccatcagttaccatttttttacctaaagaatagagaactacagctcccagaaggctgtgggggtggtgacctgtatttgaggctctctgactatatactctggaggctaatgggaggtataatttctttcatattaatttcagatatgataaaattctgtaattggctaatgtagattggtcttatgaaagggaatggaagatcagtggactttgcttttgagaaaggttaggtcagaactgggcattcttcccagcccttagtatccgttctcagtgagtataaggtgagaactacaactcccagaaggcctttgactggttagctctatatgttccagaactttgccctaggcgctgatgggggttgtagtttttcgttgatatactttaatttctatgaggagaagaaatgattgggtccctgtaagaagcttggaagtagaggatatatcctaggagaagcaggactttggggatgtttaatcatttccatctcagggtgggctaagaggaaacaaaagaagtacaactttcccagaatcaccagtgtggatcagatccctgttagccatttgcagtagctgaggagacttatacttgtgcttgtgaacctaagcaagtaatattaggcaagaaggcatcttggagtcagggtaggtgtgtcaccaagggaactggccaggcatggtggctcacacctgtaatcccagcactttaggaggccagggcaggaagactgcttgaagccaggagtttgagatcagcttgggtaacatgacaagaccccatcactacaaaagtaaaataaataaattagccgggtgtggtagtgcacgcctgtagtgccagctactcgggaggctgaggagggaggattgcttgagcccaggaggtcagggatgcaatgagctgtgattgtgccactgctctccagcctgagcgacagagagaaaccctgtctccaaaaacaaacaaacaaacaaaaaaggtgggaggggggacctcataggaacgctcttgaaacaaaaaaattgcaaaaggcaaagggtgttttcttcatttattcattcaaccacaacattgatctgaatgaacaaataaaacatgggaccctcacccccaatcccagatattgtgacccccaatgggctgaatgtgaagaagttttctgccatgcatgagttccagaacctccatgctcagagcaaggctcgaatccaggagtttgtgcggggccatttttatgggtacgtggggcatatacctaggtcttaagagaggagggcgttgggagcctaaccatctaggtctgggggagggggcagctgggggcccagactcctgggtctgagggagggggcacctgggtattcggcctccttaattcttgagcgctggaacaatgggctcaggattaaaggaaatctccatctaaagagaagaactacaactcccaggaggccccgtgatgcctctgtattacctaagacatcctctgcccccccacccatagcttctgggatttgtagttttgaggccagggctagcgggtgagggctttgggctttaccgtgccttgtgggttctttaggcatctggacttcaacttggacaagaccttatacttctttatcgccggccgctatgagttctccaacaagggtgctgacgtcttcctggaggcattggctcggctcaactatctgctcagagtgaggcctgggctatgaggggacagggagggcaagcaaagcagtttataactagggagaacagccaggcacagtggctcacacctgtaatcccaacacttttggaggccaaggcgggaggatcacttgagcccaggagatggagaccagcctgggcaacatggtgaaaccccatctctacaaaaaatacaaaaattagccaggcatggtggctcacacgtgtagtcccagctactcgggagggtgaacggggaggatcacgcgagcccgggaggtggaggttgcagtgagccaagatcatatcactgcactccagcctgggcgacagaaaaagacgctgtctcaaaaacaaaacaaaacaaaacaaaacaaaaaattacagaacagaaacccaagaagccaggaagatgggggtaggaaagcccctgagtatggtggggggccgtgaggggagtatagagacccagaaagaaggtcagagtccaggtggggactgtcctataattctccttgacaggaatgggtatggcaggatcccagtggtcttgacagaaaggctggctggttccaggcttcccccacatgatcctggcctccccctcctttctttctgcgcaggtgaacggcagcgagcagacagtggttgccttcttcatcatgccagcgcggaccaacaatttcaacgtggaaaccctcaaaggccaagctgtgcgcaaacagctttggttagcagccctcgctccggcccctggcgcagcccactcccagatgcatgctttgggtcctgtgtttctcctagagtctcagttttttgttttgtttgtttgtttgtttgtttgtttttgagacagtctcactctgtcgcccaggctggagtgtagtggtgcgatcttggctcactgcaacctccacctcctgggttcaagcaagtcatctgcctcagcctctcgagtagctgggactacaggtgcacaccaccatgcctggctaatttttgtattttcagtagagatggggtttcaccacattggccagactagtctcaaactcctgatctcgtgatctgcccgcctcagcctcccaaagtgctggaattacaggcatgagccaccgcacccggccctcttttttttttttattttgagatggagttttgctctgtcgcccaagctggagtgcaatggcatgatctcggctcactgcaacctctgcctcctgggttcaagcgattttcctgcctcagcctctgagtagctgggactacaggggtgtgccaccacacctggctaactttttatttttattagagacaggttttcactatattggccaggctggtcttgaactcctgacctcaggtgttccgcctgcctcagcctcccaaagtgctgggattccaggcgtgagccaccacacctagcctttttttgtatttttagtagagatggggtttcaccatgttggccaggctgatcttgaacacctgacctcaggtgatccagccgccttggcatcccaaagtgctgggattacaggggtgagccactgcgcctggcttagtttctttttcttttctctgatttttttttttttttgaaacagagtctcactgttgcccaggctggagtgcagtggcgtaatctcagctcactgtaacctccgcctcccagcaaagctcagtttcttcatctgtaaaaccgttcattcattagcagagatactcactgagcatctactatgtgctagctaatttttatatttttagcaccgatgggatttcaccacgttgaccaggctggtcttgaactcctgacctcaagtgacctgccctcctcatgtattccaggtcctgggaatacagctgtgaaccagaaagataagatccctttcctcatggagcttgtattctagaaaatcctgtagacggaatttacttttgggcaaaaataaagcagaactggaatatgagacggggttgtcaggacaggccacactgagaaagtgttattgggcagagacgaggaaaggtgacgagggagcaagccctgtgggtatctgaggaacagcaaactcgaaggccccgaggcatgattgagacgcggtggggagtcccatggggctggagtgacatgtctcatagagaaggggtagaaagtgaggcaagatggagggcacctgtagtgccagctacaggaggcaggaggatcattttggccctggaggttgaggctgtagtgaggcatgattgcgccactgcactccagcctaggcaacagagtgagactctgtctcaaaaaaataaaaaagaagtcgggcgtggtggctcacacctgtaatcccggcactttgggaggccgaggcgggtggatcacctgaggtcgggagtttgagaccagcttgaccaacatggtgaaaccccgtctttcctaaaaataaaaaaaatcagcagggcgtggtgacacacatctgtagtcctagctactcgagaggctaagcaggagaatcgctcgaacccgggaggcagagattgcagtgagccaagatcgcgccaatgcactccagcatgggtgacagtgagactcggtctcaaaaaaaaaaaaaaaaaaaagaaagaaagaggctgggcgcggtggctcacacctgtaatcccagcactttgggaggctgaggagggcagatcacttgaggtcaggagttcgagatcagcctggtcaacgtggtgaaatcccatcggtactaaaaatataaaaattagccagatgtggtggcgcacgcttgtaattgcagctactcaggaggctgaggcaggagaatttcttgaacctagaaagtggccgagattgcgccatcctgggcgacagagcaagactctgtctcaaaaaaaaaaaaaaaaaaaaaaaaaaaaaaaaaaaaaaaaagaaaagaaaaaagaaaaagagacaaagaaagaaaaataaatgaggccagagctatgggaggattttaagtggaggagtgaccgtggggctgttgtgtgacactgctagctttacttgtgtgcctaagtggtcctttctcagaatcttactttcttttcgtataaaatggggcttagcattgccatctgcaggacaagtacaagtcctaataatagctgacatttctggaaggcttgccatgtgtactttttggcatgacatcattgggtcctcatcgcaaacctctgaggttgattctattttcatcctcctctgaaaaatgagtcactgcagtcagagagggaaggccacctgtccctgaccacacagctgatgtcaggcacgtgggtcctaagccttccagggcctgccccctgggtccccatcctccagcctggaggccccagggcacggcttccctgctcacacctcagtttccttgctatcctagaaacctaagggtagctgggcgcggcagctcatgcctgtaattccagcactttgggaggccgaggtggccaattaccagccagaggtcaggagttcgagaccagcctggccaacgtggtgaaaccccgtctctactaaagttacaaaaattagccgggcatggtggcaggtgcctgtaatcccggctactcaggaggctgaggcaggagaattgcttgaacctgagaggcagaggttgcagtgagccaagatcgcaccactgcgctccagcctggacgacagagtaactctgtcaataataataataataataataaataaactgctgggcacagtggctcaagcctctaatcccagtactttgggaggccgaggtgggcagattacctgaggttgggagtttgagaccagcctgaccaacatggagaaaccccgtctctactaaaaatagaaaattagccgggcgcggtggctcatgcctgtaatcccagctactcgggaggctgaggcaggagaattgcttgaacctgggaggcagaggttgcagtgagccgagatcacgccattgcattccagcctgggcaacaagagccaaactccgtctcaaaataaacaaacaaactaactaaagaagcctaacagtaaatggcagctggtgtgtatgtgaccctgttgctctgcttcctccagggacacggccaacacggtgaaggaaaagttcgggaggaagctttatgaatccttactggtgtgagtgccccaccctcagccctgtgccctcctaggccctgactaacccctctcattactccgttctcacatgcatgcccctgcttgccttgcagtgggagccttcccgacatgaacaagatgctggataaggaagacttcactatgatgaagagagccatctttgcaacgcaggtatggattggacctctgtgcagagagatagcagttcctggcagccccagaggcagcttgccaggccagctcagaccccagagattgctgggagtcctgctttctcctgcagccctttatgaagggatctattctgaggtccacagaagggcacgtgctgactcgtgcaacttcagttgctggaggcgaggcacttagggtgggctgttttctgtggcttattctgccacgagggttgctgggacgtgtggtttctctgggattctggggtcaatgagcaaaaggaagcttatttgtttatttatttttattatatttatttatttattttgagatggagtctcgctctgtcgcccaggctggagtgcagtggcacgatctcggctcactgcaacctctgcctcctgggttcaagcaatactcctgccttggcctcccaagtacctgggattacaggcacatgccaccacacccagctaatttatatatttttagtagaaacgagatttcaccatgttggccaggccagtctcaagctcctgacctcaagtgttctgtctgcctcagcctcccaaagtgctgagtttacaagcgtgagtccctgttcccagcccaaagggaagtttaaaaggaagtgtagaaatgtaataaagggccaggtgtggtggctcatgcctgtaatctcagcactttgagaggccaaagcaggaagatcacttgagaccaggaggctgaggctgcagtgagccatgactgtgccactgtacaccagcctgggggacagagccagatcccacactagccccccacccacccccaaaagaaagtcaaggcccagcttggtagctcacacctgtcagtattcccagcactttgggaggctgaggtgggaggatcgcttgagcccaggagtttgagaccagcctgggcaacatgatgaaaccctgtctctaccaaaaatacaaaaatgagctgggcatagtggcatacacctgttatcccagctacctgggaggctgaggccagaggatgccttgagctcaggaggtcaaggctgcagtgagctatgattatgccgctgcagtgagctatgattatgccactgcactccagactgggcaacagagcgaggcggacacatacacacacacacgaaagaaggaaaagaaagaaaagaaaaggtcaagacagccataaaagaattacaactctcagctctccttgggccttgctgcacacggctccacctctacccctaaaactgctgggagtggtagttttatcgggccactttagggacgcagtcaggaggtgagcaaaagcacccagcattctccagaaaacattgcaaactctcagcagctgcggtttcttatttccagaggctgtgtgttggtccccagacacccctgggaattgtagctgctttagatccctaacttcagagagaattggcatttataaaatgagaacttaccgtcattctattgagtctaggggcccaaaaaactacaactcccagcagcccctgctggctttctggggcattgacgcttcccttgtccagaggcttctgggagttagagttctttcattctctggggtccagggacttcagtacttgtggtctacattggggagccatttcctggggaatggaaacccagacagctaagttgtagaaacaaaacgtgaactgcttttggctaacctataatttccaatagtccctaaagcagcccacctcattatgaatgtttctgccatcgggatgctgggagttgtagtttctcgggcccctagactcagtagagtggtggtaagtaaaatctttaatgagctatgatggaacagttgctaagaactatacaccccagcagtcctcaggacttcctatcacactagctctgtcttgctccagagactcttggaaattgtggtttctttgggcctttgttagggttggaggtgaattttcttttttcttcttttttttttttttttttttttttgagacggagtctcgctctgtcgcccaggctggagtgcagtggcgcgatcttggctcacttcaacctccgcctcccggattcatgccgttctcctgccccagcctcctgaggcacccaccaccacgcctggctaattttttgtatttttagtagagacggggtttcaccgtgttagccaggatggtctcgatctcctgacctcgtcatcctcctgcctcggcctcccagagtgctgggattacaggcgtgagccaccacgcctggtggttggaagtgaattttcataaggaaactacaactcccagcagtctccaaagcttgctgccatattggcacagtctccacccccattgactggggatagttatatttcctttgggtttctttaataatagagcccagagtacagaacaaggtagctgcaactcctcatagtccttggaggtaagggagttgctaacacggctttgcctcaaagacagctgggaattctagttctttgaagctcatggtggttaagggaaggaaaacatttccaggccaggtgtggtggctcatgtctgtaatcccagcacttcgggaggccaaaacggatgcatcgcttgagcccatgagttcgagaccaacttggccaacatagtgaaaccccatctctattaaaaaaaaaaaaaaaaaaaattaggctgagcgcaatggctcacgcctgtaatcccagcactttgggaggccaaggcaggtggatcacgaggtcaggagatctagaccatcctgatttaacacagtgaaacctcgtctctactaaaaatacaaaaaattaaccgggcgtggtggtaggcgcctgtagttccagctactcgggaggctgaggcaggagaatggcatgatcctgggaggtggagcttgcagtgagccgagattgtgccactgcattccagcctgggcgacagagcgagactccgcctcaaaaataaataataataatttaaaagaaaagaaagaaaacctttacaggttgtactagtgggaatccacaactcctggtgatcacttgggtttgttgccccacgtccggtgcatttgtggtctccttggctcccccagggtccgcatggctcatgaggaagtagggagttctctgtcccttccatgaccccttacccttcttgtggctcctgcagcggcagtctttcccccctgtgtgcacccacaatatgctggatgactcctcagaccccatcctgaccaccatccgccgaatcggcctcttcaatagcagtgccgacagggtgaaggtgagggcatttggtcagggctgggttgggcacggcagagggaaggggttgggtcctgggcctcacacactccattcccctataagttttatcaaaggtattgctgtggttagaaagaaccctggcttctgaggcaggcagacctgggtttgaacattggtcctgccaaacacatgctgtgtgatgctagacaagctttttaccctctctgggccaccttgggatctcagggcttagctaaataaggcacttgggctgagtccttctttttcactaccaggtgattttccacccggagttcctctcctccacaagccccctgctccctgtggactatgaggagtttgtccgtggctgtcaccttggagtcttcccctcctactatgagccttggggctacacaccgggtgagtgtagtgggcaggggacagcgtggtcatggcatcctagtcagagctagcatgtcttggggcgaggcactggccttctctgagcctgttgccttctttgcaaaacaggaacaatggtaatcacctgttctgctacagcgcgtgcttttgtgatacaatagctcatgtattgagaaactagaagactgggttttgaggactagtgaactggatgtcacagttcacactgctttctcccagaaagcagtggccccccaaatagtggcaatgaaatgcacaattcacaaatacagccgagtgtggccaagtctcctcgtaagacgttcccagtgcccatcaactccaccttcctcttgttggttcacattagccagtgtttttaaagctaaataaggctgggcgtggtggctcatgcctgtaatcccagcacttggggaggccaagatgggcagatcatgaggtcaggagtttgagaccagcctggccaatatagtgaaaccctgtctctactaaaaatacaaaaattagccaggtgtggtggtacgcacctgtagtctcagctacttgggaggctgaggcagagaattgcttgaacttgggaggcggaggttgcagtgagctgagatcatgccactgcactccggcagtgctgtgctctgggtgacagagcgagactccgtctcaaaaaaaaaaaaaaaaaaaaattaaagctaaattagtgccatttcctaatttattagtaatttaacaagtcctgcaaactgtgcttgcatgataattaggagtcgcggtcagccccaattccctttttccataggccttgttttatagatagattgatagatagtactagtctgttttcacaccgctataaaaaaaaaatccaagactgggtaatttataacggaaagaggtgtaattgactcaccgttccacatgtctggagaggcctcagaaaactcagtcatggcagaaggggaagaaagaagcttcttctcatggcggcaggagggagaagtatgtgagggtgcaggaaaaacgaacatttataaaaccatgagatctcgtgagaattcactcactatcatgagaacagcggcgggggaaccacccccgtaatccaatcatttccctccctcgacacgtggggactacattttgagatgaaatttgggggggaacacagagtcaaactatatcataaatatatatttaatacattttactatttgtttcttttttgagatggggttattactctgttgccaaggctcacctggaactcctaggttcaggtgcttctgcctcagcctcttgagtatctgtcattataggtgcatcccaccatgcctgacagatgtattatatttagtgcctatttttgcagtttacagatgtttttaggaaaacttacataagaattatagatcaggccaggcatggtggctcacgcctataatccccacactttgggaggccgaggtgggaggatcacttgaggacatgagtttgagaccagcctggccaacatggtgaaaccctatctctactacaaatacaaaaattagctgggggtggtggcaggcacctgtaatcccagcccttcgggaagttgaggtgggagaatcgtttgaaccccagaggcagagattgcagtgagctgagatcgcgccactgcactccagcctgggcgacagagtcagaccctgtctaaaataataataataataattataggtcaggggcagtggctcacacctataatcctagcatcttggaaggctgaggtgggaggatagcttgagctcaggagttcaagaccagcctggacaacatggtgagaccctgtctctataaaagatttttttttttttttgagacagagtcttgctctattgccaggctggagtgcagtggcacagtcttggctcactgcaaccgcctcccgggttcaagtgattctcctgcctcagcctccagagtagctgggactataggcgcctgccaccacacccagctaatttttgtatttttattagacacagggtttcaccatgttggccaggatggtttttttttttttctcttcttgacctcgtgatccacccgccttggcctcccaaagtgctgggattacagacatgagccaccgtgcccggccttttttttttttatttttaattagccatgcgtagtgttgtgtgctcgtagtcccagcctcttgggtggctgaggtgggaggattgcttgagcctaggaggtcaaggctgcagtgagctatgatcaagccactgtcctccaggttgggtgataaagtgagaccttgtctcgagaaaaaaaaaaaaaaagcattgggccaggcgcagtggctcacacctataatttgaccactttgggaagccaagacggatggatcacctgaggtcaggagttcaagacctgcctggccaacatggtgaaaccctgtctatactaaaaaatacaataattaactgggcatggtggtgtgtgcctgtagtcccagctactcgggtggctaaggcacgaaaatcgcttgaacccgggaggtgaaggttgcagtgagccgagattgtgccactgcactacagcttgggtgacagagtgagaccctgtctcgaaagaaaaaaaaaaaagcataaaaatccatcaaaaaattataatacattccttgaactatttctcaaggttgttaaaggtattaaacaagacgtactagatgctctgtacacagggatactaataatgagagaccttatcttaccccccatcaaaatagactaagtccgtacaagatactggcacaagaaaggatcttacaggttgtgacaattaagtagaatgatggaccttggcccgatcctacaggttgtgagagttaagtcaaatggatattgggatgctcaataaattagcatccttatctcccaactatggactttcccgcatcttggagcaagagcaagtaattctcttttctgagcctcttagcgtcccctctgagaaatagggaaatgcacagcctggttgtgggggctcagcgacgctccgcacagcgcctggcacttggtacaggcgccagtagagggcggtgttgaggcgtctattgcgattaagacggtaggcgatgagggaagctaagtgggtctctctcctgccttcccgcagctgagtgcacggttatgggaatccccagtatctccaccaatctctccggcttcggctgcttcatggaggaacacatcgcagacccctcagcttacggtcagcgcccaggcccctgaggattctggaagggggcttgagaacagtgactcctggatcctgggaaggaggagcttgcacccttggagaccaggggcaccaggaaatgggggggctctcgggccgctgggagaggaggggtcctggtgccacagagtcctggagaccatgaagatctccccaggatgttttctgaacctgggtcctggcctccacaggtatctacattcttgaccggcggttccgcagcctggatgattcctgctcgcagctcacctccttcctctacagtttctgtcagcagagccggcggcagcgtatcatccagcggaaccgcacggagcgcctctccgaccttctggactggaaatacctaggccgggtaggacccccactttcctatcctctcctggcaaatcagcagctcctggctaggggtctttggtgcagggtgcagggtctcatcctacttgtaacagttgctgtccctttaagcaatgaaccaggccgatcgcgatggtggctcatggttgtaatcccagcactttgggaggcctaacaggaaggatcgcatgagctcaggagttaagagaccagcctgggcaacattttatttttactaaaaataaaaaaaaatcagccgagcgtgatggtgcgtgcctgtagtcccagctactggggaggctgaggtggtagggtcgcttgagccccggaggttgaggctgcagtgagccttgttcaaacccactgcactccatcctagagtaacagaacaagaccgtgtttaaaaagaaaaaaagaacattggtcaggcgttctcatttgcataagggtgtggccaaagaggagactcacttgagaattcggcccctgtttgaggggtatggctcagaaactgtccattcacaggtctgcttatctacataaggggtggagtcagatagatggattatttgtatggggtgcagcagtgtgctcatctgcataaagggtgtggccaagacccccatcatctgcctgggggtggggcctggctctcacatcctctgccccctctcctcctttcccctagtactatatgtctgcgcgccacatggcgctgtccaaggcctttccagagcacttcacctacgagcccaacgaggcggatgcggtgagtggaccctggatttctgcttagccaggagctaaagggctgggcttcggtggggtgggggtccctcctggagtgggaaggcctagaggtggctggatgcctgggtgggactgtgaggtcctcagctcaccctggtttgcacccacatcgccgtatgcaggcccaggggtaccgctacccacggccagcctcggtgccaccgtcgccctcgctgtcacgacactccagcccgcaccagagtgaggacgaggaggatccccggaacgggccgctggaggaagacggcgagcgctacgatgaggacgaggaggccgccaaggaccggcgcaacatccgtgcaccagagtggccgcgccgagcgtcctgcacctcctccaccagcggcagcaagcgcaactctgtggacacggccacctccagctcactcagcaccccgagcgagcccctcagccccaccagctccctgggcgaggagcgtaactaagtccgccccaccacactccccgcctgtcctgcctctctgctccagagagaggatgcagaggggtgctgctcctaaacccccgctccagatctgcactgggtgtggccccgcagtgcccccacccagtccgccaaacactccaccccctccagctccagtttccaagttcctgcactccagaatccacaaagccgtgcctttctctggctccagaatatgcataatcagcgccctggagtcccctgggcctggaccgcttcccagaggccaggaatctgccattactctgcggtggtgccagaggttttaggaaacctggcatggtgctttcaggtctggggcttttagagccccccgtgtggcttacaaattctacagcatacagagcaggccacgctcaggcccggcatgcgggccaccaagttctggaaaccacgtggtgtccctgcgaatggggcgatcaagtccagagccggggcactttcagagtttgaaggtaactgagagcagatggtcctccatttcaactccagaagtggggctctgggagggatgttctagccctccctggcatgtcagagccaggctctgcctggaggatccctccatccggctcctgtcatcccctacactttggccaagcaagaggtggtagaaccacttggctgctcattccttctggaggacacacagtctcagtccagatgccttcctgtctttctggccctttctggaccagatcctactcttcctttctaaatctgagatctccctccagggaatccgcctgcagaggacagagctggctgtcttcccccacccctaacctggcttattcccaactgctctgcccactgtgaaaccactaggttctaggtcctggcttctagatctggaaccttaccacgttactgcatactgatccctttcccatgatccagaactgaggtcactgggttctagaacccccacatttacctcgaggctcttccatccccaaactgtgccctgccttcagctttggtgaaagggagggcccctcatgtgtgctgtgctgtgtctgcaccgcttggtttgcagttgagaggggagggcaggaggggtgtgattggagtgtgtccggagatgagatgaaaaaaatacatctatatttaagaa

SEQ ID NO: 12 - DNA, 3569 bp, Homo sapiens
  source 1..3569: mol_type = other DNA
agtgacgctgcggcctccttctgcctaggtcccaacgcttcggggcaggggtgcggtcttgcaataggaagccgagcgtcttgcaagcttcccgtcgggcaccagctactcggccccgcaccctacctggtgcattccctagacacctccggggtccctacctggagatccccggagccccccttcctgcgccagccatgcctttaaaccgcactttgtccatgtcctcactgccaggactggaggactgggaggatgaattcgacctggagaacgcagtgctcttcgaagtggcctgggaggtggctaacaaggtgggtggcatctacacggtgctgcagacgaaggcgaaggtgacaggggacgaatggggcgacaactacttcctggtggggccgtacacggagcagggcgtgaggacccaggtggaactgctggaggcccccaccccggccctgaagaggacactggattccatgaacagcaagggctgcaaggtgtatttcgggcgctggctgatcgagggaggccctctggtggtgctcctggacgtgggtgcctcagcttgggccctggagcgctggaagggagagctctgggatacctgcaacatcggagtgccgtggtacgaccgcgaggccaacgacgctgtcctctttggctttctgaccacctggttcctgggtgagttcctggcacagagtgaggagaagccacatgtggttgctcacttccatgagtggttggcaggcgttggactctgcctgtgtcgtgcccggcgactgcctgtagcaaccatcttcaccacccatgccacgctgctggggcgctacctgtgtgccggtgccgtggacttctacaacaacctggagaacttcaacgtggacaaggaagcaggggagaggcagatctaccaccgatactgcatggaaagggcggcagcccactgcgctcacgtcttcactactgtgtcccagatcaccgccatcgaggcacagcacttgctcaagaggaaaccagatattgtgacccccaatgggctgaatgtgaagaagttttctgccatgcatgagttccagaacctccatgctcagagcaaggctcgaatccaggagtttgtgcggggccatttttatgggcatctggacttcaacttggacaagaccttatacttctttatcgccggccgctatgagttctccaacaagggtgctgacgtcttcctggaggcattggctcggctcaactatctgctcagagtgaacggcagcgagcagacagtggttgccttcttcatcatgccagcgcggaccaacaatttcaacgtggaaaccctcaaaggccaagctgtgcgcaaacagctttgggacacggccaacacggtgaaggaaaagttcgggaggaagctttatgaatccttactggttgggagccttcccgacatgaacaagatgctggataaggaagacttcactatgatgaagagagccatctttgcaacgcagcggcagtctttcccccctgtgtgcacccacaatatgctggatgactcctcagaccccatcctgaccaccatccgccgaatcggcctcttcaatagcagtgccgacagggtgaaggtgattttccacccggagttcctctcctccacaagccccctgctccctgtggactatgaggagtttgtccgtggctgtcaccttggagtcttcccctcctactatgagccttggggctacacaccggctgagtgcacggttatgggaatccccagtatctccaccaatctctccggcttcggctgcttcatggaggaacacatcgcagacccctcagcttacggtatctacattcttgaccggcggttccgcagcctggatgattcctgctcgcagctcacctccttcctctacagtttctgtcagcagagccggcggcagcgtatcatccagcggaaccgcacggagcgcctctccgaccttctggactggaaatacctaggccggtactatatgtctgcgcgccacatggcgctgtccaaggcctttccagagcacttcacctacgagcccaacgaggcggatgcggcccaggggtaccgctacccacggccagcctcggtgccaccgtcgccctcgctgtcacgacactccagcccgcaccagagtgaggacgaggaggatccccggaacgggccgctggaggaagacggcgagcgctacgatgaggacgaggaggccgccaaggaccggcgcaacatccgtgcaccagagtggccgcgccgagcgtcctgcacctcctccaccagcggcagcaagcgcaactctgtggacacggccacctccagctcactcagcaccccgagcgagcccctcagccccaccagctccctgggcgaggagcgtaactaagtccgccccaccacactccccgcctgtcctgcctctctgctccagagagaggatgcagaggggtgctgctcctaaacccccgctccagatctgcactgggtgtggccccgcagtgcccccacccagtccgccaaacactccaccccctccagctccagtttccaagttcctgcactccagaatccacaaagccgtgcctttctctggctccagaatatgcataatcagcgccctggagtcccctgggcctggaccgcttcccagaggccaggaatctgccattactctgcggtggtgccagaggttttaggaaacctggcatggtgctttcaggtctggggcttttagagccccccgtgtggcttacaaattctacagcatacagagcaggccacgctcaggcccggcatgcgggccaccaagttctggaaaccacgtggtgtccctgcgaatggggcgatcaagtccagagccggggcactttcagagtttgaaggtaactgagagcagatggtcctccatttcaactccagaagtggggctctgggagggatgttctagccctccctggcatgtcagagccaggctctgcctggaggatccctccatccggctcctgtcatcccctacactttggccaagcaagaggtggtagaaccacttggctgctcattccttctggaggacacacagtctcagtccagatgccttcctgtctttctggccctttctggaccagatcctactcttcctttctaaatctgagatctccctccagggaatccgcctgcagaggacagagctggctgtcttcccccacccctaacctggcttattcccaactgctctgcccactgtgaaaccactaggttctaggtcctggcttctagatctggaaccttaccacgttactgcatactgatccctttcccatgatccagaactgaggtcactgggttctagaacccccacatttacctcgaggctcttccatccccaaactgtgccctgccttcagctttggtgaaagggagggcccctcatgtgtgctgtgctgtgtctgcaccgcttggtttgcagttgagaggggagggcaggaggggtgtgattggagtgtgtccggagatgagatgaaaaaaatacatctatatttaagaa

SEQ ID NO: 13 - DNA, 3377 bp, Homo sapiens
  source 1..3377: mol_type = other DNA
agtgacgctgcggcctccttctgcctaggtcccaacgcttcggggcaggggtgcggtcttgcaataggaagccgagcgtcttgcaagcttcccgtcgggcaccagctactcggccccgcaccctacctggtgcattccctagacacctccggggtccctacctggagatccccggagccccccttcctgcgccagccatgcctttaaaccgcactttgtccatgtcctcactgccaggactggaggactgggaggatgaattcgacctggagaacgcagtgctcttcgaagtggcctgggaggtggctaacaaggtgggtggcatctacacggtgctgcagacgaaggcgaaggtgacaggggacgaatggggcgacaactacttcctggtggggccgtacacggagcagggcgtgaggacccaggtggaactgctggaggcccccaccccggccctgaagaggacactggattccatgaacagcaagggctgcaagttcctggcacagagtgaggagaagccacatgtggttgctcacttccatgagtggttggcaggcgttggactctgcctgtgtcgtgcccggcgactgcctgtagcaaccatcttcaccacccatgccacgctgctggggcgctacctgtgtgccggtgccgtggacttctacaacaacctggagaacttcaacgtggacaaggaagcaggggagaggcagatctaccaccgatactgcatggaaagggcggcagcccactgcgctcacgtcttcactactgtgtcccagatcaccgccatcgaggcacagcacttgctcaagaggaaaccagatattgtgacccccaatgggctgaatgtgaagaagttttctgccatgcatgagttccagaacctccatgctcagagcaaggctcgaatccaggagtttgtgcggggccatttttatgggcatctggacttcaacttggacaagaccttatacttctttatcgccggccgctatgagttctccaacaagggtgctgacgtcttcctggaggcattggctcggctcaactatctgctcagagtgaacggcagcgagcagacagtggttgccttcttcatcatgccagcgcggaccaacaatttcaacgtggaaaccctcaaaggccaagctgtgcgcaaacagctttgggacacggccaacacggtgaaggaaaagttcgggaggaagctttatgaatccttactggttgggagccttcccgacatgaacaagatgctggataaggaagacttcactatgatgaagagagccatctttgcaacgcagcggcagtctttcccccctgtgtgcacccacaatatgctggatgactcctcagaccccatcctgaccaccatccgccgaatcggcctcttcaatagcagtgccgacagggtgaaggtgattttccacccggagttcctctcctccacaagccccctgctccctgtggactatgaggagtttgtccgtggctgtcaccttggagtcttcccctcctactatgagccttggggctacacaccggctgagtgcacggttatgggaatccccagtatctccaccaatctctccggcttcggctgcttcatggaggaacacatcgcagacccctcagcttacggtatctacattcttgaccggcggttccgcagcctggatgattcctgctcgcagctcacctccttcctctacagtttctgtcagcagagccggcggcagcgtatcatccagcggaaccgcacggagcgcctctccgaccttctggactggaaatacctaggccggtactatatgtctgcgcgccacatggcgctgtccaaggcctttccagagcacttcacctacgagcccaacgaggcggatgcggcccaggggtaccgctacccacggccagcctcggtgccaccgtcgccctcgctgtcacgacactccagcccgcaccagagtgaggacgaggaggatccccggaacgggccgctggaggaagacggcgagcgctacgatgaggacgaggaggccgccaaggaccggcgcaacatccgtgcaccagagtggccgcgccgagcgtcctgcacctcctccaccagcggcagcaagcgcaactctgtggacacggccacctccagctcactcagcaccccgagcgagcccctcagccccaccagctccctgggcgaggagcgtaactaagtccgccccaccacactccccgcctgtcctgcctctctgctccagagagaggatgcagaggggtgctgctcctaaacccccgctccagatctgcactgggtgtggccccgcagtgcccccacccagtccgccaaacactccaccccctccagctccagtttccaagttcctgcactccagaatccacaaagccgtgcctttctctggctccagaatatgcataatcagcgccctggagtcccctgggcctggaccgcttcccagaggccaggaatctgccattactctgcggtggtgccagaggttttaggaaacctggcatggtgctttcaggtctggggcttttagagccccccgtgtggcttacaaattctacagcatacagagcaggccacgctcaggcccggcatgcgggccaccaagttctggaaaccacgtggtgtccctgcgaatggggcgatcaagtccagagccggggcactttcagagtttgaaggtaactgagagcagatggtcctccatttcaactccagaagtggggctctgggagggatgttctagccctccctggcatgtcagagccaggctctgcctggaggatccctccatccggctcctgtcatcccctacactttggccaagcaagaggtggtagaaccacttggctgctcattccttctggaggacacacagtctcagtccagatgccttcctgtctttctggccctttctggaccagatcctactcttcctttctaaatctgagatctccctccagggaatccgcctgcagaggacagagctggctgtcttcccccacccctaacctggcttattcccaactgctctgcccactgtgaaaccactaggttctaggtcctggcttctagatctggaaccttaccacgttactgcatactgatccctttcccatgatccagaactgaggtcactgggttctagaacccccacatttacctcgaggctcttccatccccaaactgtgccctgccttcagctttggtgaaagggagggcccctcatgtgtgctgtgctgtgtctgcaccgcttggtttgcagttgagaggggagggcaggaggggtgtgattggagtgtgtccggagatgagatgaaaaaaatacatctatatttaagaa

SEQ ID NO: 14 - DNA, 106 bp, synthetic construct
  source 1..106: mol_type = other DNA
ctgcgcgctcgctcgctcactgaggccgccggcccaaagggccggcgtcgggcgacctttggtcgcccggcctcagtgagcgagcgagcgcgcagagagggagtgg

SEQ ID NO: 15 - DNA, 145 bp, synthetic construct
  source 1..145: mol_type = other DNA
aggaacccctagtgatggagttggccactccctctctgcgcgctcgctcgctcactgaggccgggcgaccaaaggtcgcccgacgccggccctttgggccggcggcctcagtgagcgagcgagcgcgcagagagggagtggccaa

SEQ ID NO: 16 - DNA, 106 bp, synthetic construct
  source 1..106: mol_type = other DNA
ctgcgcgctcgctcgctcactgaggccgcccgggcaaagcccgggcgtcgggcgacctttggtcgcccggcctcagtgagcgagcgagcgcgcagagagggagtgg

SEQ ID NO: 17 - DNA, 170 bp, synthetic construct
  source 1..170: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttc

SEQ ID NO: 18 - DNA, 104 bp, synthetic construct
  source 1..104: mol_type = other DNA
tgcgcgctcgctcgctcactgaggccgcccgggcaaagcccgggcgtcgggcgacctttggtcgcccggcctcagtgagcgagcgagcgcgcagagagggagtg

SEQ ID NO: 19 - DNA, 106 bp, synthetic construct; identical to SEQ ID NO: 14
  source 1..106: mol_type = other DNA

SEQ ID NO: 20 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 21 - DNA, 145 bp, synthetic construct
  source 1..145: mol_type = other DNA
aggaacccctagtgatggagttggccactccctctctgcgcgctcgctcgctcactgaggccgccggcccaaagggccggcgtcgggcgacctttggtcgcccggcctcagtgagcgagcgagcgcgcagagagggagtggccaa

SEQ ID NO: 22 - DNA, 145 bp, synthetic construct; identical to SEQ ID NO: 15
  source 1..145: mol_type = other DNA

SEQ ID NO: 23 - DNA, 164 bp, synthetic construct
  source 1..164: mol_type = other DNA
gggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgt

SEQ ID NO: 24 - DNA, 225 bp, synthetic construct
  source 1..225: mol_type = other DNA
caattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaa

SEQ ID NO: 25 - DNA, 426 bp, synthetic construct
  source 1..426: mol_type = other DNA
ataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaa

SEQ ID NO: 26 - DNA, 793 bp, synthetic construct
  source 1..793: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcag

SEQ ID NO: 27 - DNA, 241 bp, synthetic construct
  source 1..241: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 28 - DNA, 105 bp, synthetic construct
  source 1..105: mol_type = other DNA
cgcggaacccctatttgtttatttttctaaatacattcaaatatgtatccgctcatgagacaataaccctgataaatgcttcaataatattgaaaaaggaagagt

SEQ ID NO: 29 - DNA, 123 bp, synthetic construct
  source 1..123: mol_type = other DNA
tgtttattgcagcttataatggttacaaataaagcaatagcatcacaaatttcacaaataaagcatttttttcactgcattctagttgtggtttgtccaaactcatcaatgtatcttatcatg

SEQ ID NO: 30 - DNA, 254 bp, synthetic construct
  source 1..254: mol_type = other DNA
ctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaacagcaggcatgctggggatgcggtgggctctatggcttctgaggcggaaagaaccagct

SEQ ID NO: 31 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaacagcaggcatgctggggatgcggtgggctctatgg

SEQ ID NO: 32 - DNA, 105 bp, synthetic construct; identical to SEQ ID NO: 28
  source 1..105: mol_type = other DNA

SEQ ID NO: 33 - DNA, 816 bp, synthetic construct
  source 1..816: mol_type = other DNA
atgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgtgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 34 - DNA, 589 bp, synthetic construct
  source 1..589: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 35 - DNA, 21 bp, synthetic construct
  source 1..21: mol_type = other DNA
aaaacttcttcacattcagcc

SEQ ID NO: 36 - DNA, 21 bp, synthetic construct
  source 1..21: mol_type = other DNA
taaggattcataaagcttcct

SEQ ID NO: 37 - DNA, 21 bp, synthetic construct
  source 1..21: mol_type = other DNA
tcatcatagtgaagtcttcct

SEQ ID NO: 38 - DNA, 21 bp, synthetic construct
  source 1..21: mol_type = other DNA
aaggattcataaagcttcctc

SEQ ID NO: 39 - DNA, 21 bp, synthetic construct
  source 1..21: mol_type = other DNA
agtgtcgtgacagcgagggcg

SEQ ID NO: 40 - RNA, 22 nt, synthetic construct
  source 1..22: mol_type = other RNA
tacgttgaaattattggtccgg

SEQ ID NO: 41 - RNA, 22 nt, synthetic construct
  source 1..22: mol_type = other RNA
gattcgtagagcttcctcccaa

SEQ ID NO: 42 - DNA, 20 bp, synthetic construct
  source 1..20: mol_type = other DNA
cagagcaaagcacgaatcca

SEQ ID NO: 43 - DNA, 20 bp, synthetic construct
  source 1..20: mol_type = other DNA
catagcggccagcgataaag

SEQ ID NO: 44 - DNA, 16 bp, synthetic construct
  source 1..16: mol_type = other DNA
ttatgggcacctggac

SEQ ID NO: 45 - DNA, 26 bp, synthetic construct
  source 1..26: mol_type = other DNA
tgcctaatatacctgtgcaaacaatc

SEQ ID NO: 46 - DNA, 24 bp, synthetic construct
  source 1..24: mol_type = other DNA
ttccttccatttttccaaatagct

SEQ ID NO: 47 - DNA, 17 bp, synthetic construct
  source 1..17: mol_type = other DNA
caagagctgctgcagaa

SEQ ID NO: 48 - DNA, 20 bp, synthetic construct
  source 1..20: mol_type = other DNA
cccttacgccaagactatgc

SEQ ID NO: 49 - DNA, 20 bp, synthetic construct
  source 1..20: mol_type = other DNA
tggaacaaccttctcggatt

SEQ ID NO: 50 - DNA, 20 bp, synthetic construct
  source 1..20: mol_type = other DNA
aagtgctgccgtcattttct

SEQ ID NO: 51 - DNA, 20 bp, synthetic construct
  source 1..20: mol_type = other DNA
ataggctcgcagggatgatt

SEQ ID NO: 52 - DNA, 20 bp, synthetic construct
  source 1..20: mol_type = other DNA
tgccaacccagagaagaagt

SEQ ID NO: 53 - DNA, 20 bp, synthetic construct
  source 1..20: mol_type = other DNA
agcaagcaatgacagggaag

SEQ ID NO: 54 - DNA, 20 bp, synthetic construct
  source 1..20: mol_type = other DNA
gcctcaaggacgacaacatc

SEQ ID NO: 55 - DNA, 20 bp, synthetic construct
  source 1..20: mol_type = other DNA
cacactcaccacccattcag

SEQ ID NO: 56 - DNA, 20 bp, synthetic construct
  source 1..20: mol_type = other DNA
ctgtgtgggtggatgtgaag

SEQ ID NO: 57 - DNA, 20 bp, synthetic construct
  source 1..20: mol_type = other DNA
tcctgagtgtcgtttgttgc

SEQ ID NO: 58 - DNA, 23 bp, synthetic construct
  source 1..23: mol_type = other DNA
ccgctatgagttctccaacaagg

SEQ ID NO: 59 - DNA, 22 bp, synthetic construct
  source 1..22: mol_type = other DNA
agaaggcaaccactgtctgctc

SEQ ID NO: 60 - DNA, 18 bp, synthetic construct
  source 1..18: mol_type = other DNA
gccacatcgctcagacac

SEQ ID NO: 61 - DNA, 23 bp, synthetic construct
  source 1..23: mol_type = other DNA
ggcaacaatatccactttaccag

SEQ ID NO: 62 - RNA, 22 nt, synthetic construct
  source 1..22: mol_type = other RNA
ggcctggttattaaagttgcat

SEQ ID NO: 63 - RNA, 37 nt, synthetic construct
  source 1..37: mol_type = other RNA
catgccggcccggaccaataatttcaacgtggaaacc

(19) United States
(12) Patent Application Publication
(10) Pub. No.: US 2025/0011791 A1
(43) Pub. Date: Jan. 9, 2025

(54) RNAI THERAPY FOR APBD AND LAFORA DISEASE

(71) Applicant: THE BOARD OF REGENTS OF THE UNIVERSITY OF TEXAS SYSTEM, Austin, TX (US)

(72) Inventors: Berge A. MINASSIAN, Austin, TX (US); Emrah GUMUSGOZ, Austin, TX (US); Mayank VERMA, Austin, TX (US)

(73) Assignee: THE BOARD OF REGENTS OF THE UNIVERSITY OF TEXAS SYSTEM, Austin, TX (US)

(21) Appl. No.: 18/709,305

(22) PCT Filed: Nov. 17, 2022
(86) PCT No.: PCT/US2022/080028

§ 371 Date: May 10, 2024

Related U.S. Application Data
(60) Provisional application No. 63/281,336, filed on Nov. 19, 2021.

(51) Int. Cl. C12N 15/113; A61P 3/00; A61P 25/28; C12N 15/86

(52) U.S. Cl. CPC C12N 15/1137; A61P 3/00; A61P 25/28; C12N 15/86; C12N 2310/141; C12N 2750/14143; C12Y 204/01011

(57) ABSTRACT

The present disclosure provides methods and compositions for the treatment of glycogen storage disorders, such as, for example, Lafora Disease and adult polyglucosan body disease. The methods and compositions of the present disclosure comprise isolated nucleic acid molecules, rAAV vectors and rAAV viral vectors comprising polynucleotide sequences encoding for artificial micro RNAs (amiRNAs) directed against GYS1.

### CROSS-REFERENCE TO RELATED APPLICATIONS

The present application claims the benefit of U.S. Provisional Patent Application No. 63/281,336 filed Nov. 19, 2021, which is incorporated herein by reference in its entirety.

### INCORPORATION-BY-REFERENCE OF SEQUENCE LISTING

The Sequence Listing XML associated with this application is provided electronically in XML file format and is hereby incorporated by reference into the specification. The name of the XML file containing the Sequence Listing XML is “TAYS-018_001WO_SeqList_ST26”. The XML file is 112,626 bytes, created on Nov. 17, 2022 and is being submitted electronically via USPTO Patent Center.

### FIELD

The present disclosure provides methods and compositions for the treatment of glycogen storage disorders, such as, for example, Lafora Disease and adult polyglucosan body disease. The methods and compositions of the present disclosure comprise isolated nucleic acid molecules, rAAV vectors and rAAV viral vectors comprising polynucleotide sequences encoding for artificial micro RNAs (amiRNAs) directed against GYS1.

### BACKGROUND

Adult polyglucosan body disease (APBD) and Lafora disease (LD) are autosomal recessive glycogen storage neurological disorders. Adult polyglucosan body disease (APBD) is a fatal adult-onset neurodegenerative disorder characterized by progressive sensory deficits, upper and lower motor neuron signs, ataxia, loss of urinary bladder control, and premature death. It is associated with mutations in the GBE1 gene, which encodes the glycogen branching enzyme (GBE) (Lossos et al., Ann Neurol. 1998; 44(6):867-72; Akman et al., JAMA Neurol. 2015; 72(4):441-5). This is one of the two main enzymes that synthesize glycogen, the other being glycogen synthase. As soon as glycogen synthase elongates a glycogen branch, GBE cleaves this extension, en bloc, and reattaches it upstream, converting the linear glucan to a fork. Glycogen synthase thus elongates each prong of the fork, which GBE branches, and so forth. The final macromolecule, the largest molecule in the cytosol, occupies a spherical space with all the branches pointing away from each other, allowing it to be perfused with water and thus soluble (Roach et al., Curr Mol Med. 2002; 2(2):101-20). In APBD, GBE cannot keep pace with glycogen synthase, and glucan chains become too long. Glycogen with overlong branches (termed polyglucosan) precipitates, possibly because long branches wind around each other and extrude water, as in plant starches (Sullivan et al., Cell Rep. 2019; 27(5):1334-44 e6). Polyglucosans aggregate, and accumulate into polyglucosan bodies (PBs), which provoke neuroinflammation and neurodegeneration (Lossos 1998; Akman 2015; Gumusgoz et al., Neurotherapeutics. 2021; 18(2):1414-25). PBs form in glia and neurons, in the latter in axons, which they appear to clog, perhaps explaining the disease's axonopathic neurological presentation (Robitaille et al., Brain. 1980; 103(2):315-36).

Lafora disease (LD) is a fatal teenage-onset progressive myoclonus epilepsy. It is caused by mutations in the EPM2A or EPM2B genes, which encode the laforin glycogen phosphatase and its interacting malin E3 ubiquitin ligase (Minassian et al., Nat Genet. 1998; 20(2):171-4; Chan et al., Nat Genet. 2003; 35(2):125-7). The precise functions of laforin and malin are not known, but loss of either leads to overlong glycogen branches and profuse PB formation very similar to APBD, minus the peculiar axonal localization. As in APBD, there is neuroinflammation and neurodegeneration (Nitschke et al., Nat Rev Neurol. 2018; 14(10):606-17; Lahuerta et al., Mol Neurobiol. 2020; 57(3):1607-21; Muccioli et al., Eur J Nucl Med Mol Imaging. 2020; 47(6):1576-84; Van Heycop Ten Ham et al., The Epilepsies Handbook of Clinical Neurology 15. Amsterdam, New York: North-Holland Publishing Elsevier; 1974. p. 382-422; Lafora et al., Z Ges Neurol Psychiat. 1911; 6:1-14). LD is further characterized by a severe epilepsy (Ortolano et al., Mol Brain. 2014; 7:7; Valles-Ortega et al., EMBO Mol Med. 2011; 3(11):667-81).

Glycogen synthase was downregulated in mouse models of APBD and LD by targeting of the glycogen synthase gene Gys1, or of genes (Ppp1r3c, Ppp1r3d) that encode glycogen synthase activating enzymes. In all cases, PB formation was prevented or halted, neuropathological features corrected, and, where tested, behavioral, motor or epileptic abnormalities rescued (Gumusgoz 2021; Pederson 2013; Duran 2014; Varea 2021; Chown 2020; Israelian 2021; Nitschke 2021; Turnbull 2011; Turnbull 2014; Ahonen 2021).

There are currently no treatments for LD or APBD, and therapeutic intervention is limited to treating specific symptoms. Thus, there is a need in the art for compositions and methods directed to the treatment of LD and/or APBD, including treatments directed at decreasing Gys1 expression in a subject so as to downregulate glycogen synthase expression.

### SUMMARY

The present disclosure relates generally to the field of RNA interference (RNAi) and in particular, to recombinant adeno-associated viral (AAV) vector particles (also known as rAAV viral vectors) comprising sequences encoding artificial micro RNA molecules (amiRNAs) targeting the glycogen synthase 1 gene (GYS1; NCBI Reference Sequence: NG_012923.1; SEQ ID NO: 11), their manufacture, and their use to deliver amiRNA-encoding polynucleotide sequences to treat or prevent a disease or disorder, including, for example, LD and APBD.

In one aspect, provided herein is an rAAV vector comprising at least one polynucleotide sequence encoding at least one amiRNA directed against GYS1, wherein the at least one polynucleotide comprises the nucleic acid sequences set forth in any one of SEQ ID NOS: 1-5. In some embodiments, the rAAV vector further comprises a first AAV ITR sequence. In some embodiments, the first AAV ITR sequence is an AAV2 ITR sequence. In some embodiments, the first AAV ITR sequence comprises the sequence set forth in SEQ ID NO: 14. In some embodiments, the rAAV vector further comprises a second AAV ITR sequence. In some embodiments, the second AAV ITR sequence is an AAV2 ITR sequence. In some embodiments, the second AAV ITR sequence comprises the sequence set forth in SEQ ID NO: 15.

In some embodiments, the rAAV vector further comprises a promoter sequence. In some embodiments, the promoter sequence is a CBh promoter sequence. In some embodiments, the CBh promoter sequence comprises the nucleic acid sequence set forth in SEQ ID NO: 26.

In another aspect, provided herein is an rAAV vector comprising, in the 5′ to 3′ direction: a first AAV2 ITR sequence; a CBh promoter sequence; a polynucleotide sequence encoding for at least one amiRNA directed against GYS1; and a second AAV2 ITR sequence. In some embodiments, the rAAV vector comprises the sequence set forth in any one of SEQ ID NOs: 6-10.

In another aspect, provided herein is an rAAV viral vector comprising an AAV capsid protein; and an rAAV vector described herein. In some embodiments, the AAV capsid protein is an AAV9 capsid protein.

In another aspect, provided herein is a pharmaceutical composition comprising an rAAV vector or an rAAV viral vector and at least one pharmaceutically acceptable excipient and/or additive.

In another aspect, provided herein is a method for treating a subject having a glycogen storage disorder, the method comprising administering to the subject a therapeutically effective amount of an rAAV vector, an rAAV viral vector, or a pharmaceutical composition provided herein. In some embodiments, the rAAV vector, the rAAV viral vector, or the pharmaceutical composition is administered to the subject at a dose ranging from about 10¹¹ to about 10¹⁸ viral vector particles. In some embodiments, the rAAV vector, the rAAV viral vector, or pharmaceutical composition is administered intravenously. In some embodiments, the glycogen storage disorder is Lafora Disease or adult polyglucosan body disease.

In another aspect, provided herein is an rAAV vector, an rAAV viral vector, or a pharmaceutical composition described herein for use in the treatment of a glycogen storage disorder.

In some embodiments, the rAAV vector, the rAAV viral vector, or the pharmaceutical composition is for administration to a subject at a dose ranging from about 10¹¹ to about 10¹⁸ viral vector particles. In some embodiments, the rAAV vector, the rAAV viral vector, or pharmaceutical composition is for intravenous administration. In some embodiments, the glycogen storage disorder is Lafora Disease or adult polyglucosan body disease.

### BRIEF DESCRIPTION OF THE DRAWINGS

The above and further features will be more clearly appreciated from the following detailed description when taken in conjunction with the accompanying drawings.

FIGS. 1A-1C: AAV-amiRNA targeting murine Gys1 mRNA. Schematic representation of AAV-amiRNA and target site sequences (FIG. 1A). GCCUGGUUAUUAAAGUUGCAU (SEQ ID NO: 62) and CAUGCCGGCCCGGACCAAUAAUUUCAACGUGGAAACC (SEQ ID NO: 63). AAV-amiRNA vector components: CBh, hybrid Chicken Beta Actin promoter; GFP, enhanced green fluorescent protein; bGH-pA, bovine growth hormone polyadenylation signal (FIG. 1B). Representative images show GFP expression in sagittal brain sections of AAV-amiRNA treated (FIG. 1C) vs PBS treated mice (inset of FIG. 1C). Scale bar, 2 mm.

FIGS. 2A-2I: Gys1 targeting AAV-amiRNA reduces GYS1 mRNA and protein levels, insoluble glycogen and PB accumulation in brains of the GbelY239S APBD mouse model. Gys1 mRNA level (FIG. 2A) was measured by ddPCR. Representative brain GYS1 western blots with stain-free gel as loading control (FIG. 2B). Quantification of GYS1 western blots normalized to stain-free gel shown in (FIG. 2C). PB quantification in the hippocampus (FIG. 2D) and whole-brain degradation-resistant glycogen content (FIG. 2E). Representative micrographs of PASD stained hippocampus of PBS (FIGS. 2F and 2H) vs AAV-amiRNA (FIGS. 2G and 2I) treated mice. Scale bar, 300 μm. Data are presented as mean±SEM. Significance levels are indicated as *, p<0.05; **, p<0.01; ***, p<0.001; ****, and p<0.0001.

FIGS. 3A-3I: Gys1 targeting AAV-amiRNA reduces Gys1 mRNA and protein levels, insoluble glycogen and PB accumulation in brains of the Epm2a−/− LD mouse model. Gys1 mRNA level (FIG. 3A) was measured by ddPCR. Representative brain GYS1 western blots with stain-free gel as loading control (FIG. 3B). Quantification of GYS1 western blots normalized to stain-free gel shown in (FIG. 3C). PB quantification in the hippocampus (FIG. 3D) and whole-brain degradation-resistant glycogen content (FIG. 3E). Representative micrographs of PASD stained hippocampus of PBS (FIGS. 3F and 3H) vs AAV-amiRNA (FIGS. 3G and 3I) treated mice. Scale bar, 300 μm. Data are presented as mean±SEM. Significance levels are indicated as *, p<0.05; **, p<0.01; ***, p<0.001; ****, and p<0.0001.

FIGS. 4A-4I: Gys1 targeting AAV-amiRNA reduces Gys1 mRNA and protein levels, insoluble glycogen and PB accumulation in brains of the Epm2b−/− LD mouse model. Gys1 mRNA level (FIG. 4A) was measured by ddPCR. Representative brain GYS1 Western blots with stain-free gel as loading control (FIG. 4B). Quantification of GYS1 Western blots normalized to stain-free gel shown in (FIG. 4C). PB quantification in the hippocampus (FIG. 4D) and degradation-resistant glycogen content (FIG. 4E). Representative micrographs of PASD stained hippocampus of PBS (FIGS. 4F and 4H) vs AAV-amiRNA (FIGS. 4G and 4I) treated mice. Scale bar, 300 μm. Data are presented as mean±SEM. Significance levels are indicated as *, p<0.05; **, p<0.01; ***, p<0.001; ****, and p<0.0001.

FIGS. 5A-5C: AAV-amiRNA ameliorates PB-associated immune activation. “WT” indicates the wild type control group. For each panel, from left to right, Cxcl10, Ccl5, Lcn2 and C3 were used as neuroinflammation markers, and relative mRNA expression levels were analyzed by qRT-PCR for Gbe1Y239S (FIG. 5A), Epm2a−/− (FIG. 5B) and Epm2b−/− (FIG. 5C) mice. In FIG. 5A: WT (N=13), PBS (N=12) and AAV-amiRNA (N=11). In FIG. 5B: WT (N=8), PBS (N=13), and AAV-amiRNA (N=12). In FIG. 5C: WT (N=11), PBS (N=10), and AAV-amiRNA (N=13). Data are presented as mean±SEM. Significance levels are indicated as *, p<0.05; **, p<0.01; ***, p<0.001, ****, and p<0.0001.

FIG. 6 shows an illustrative AAV-amiRNA vector.

FIG. 7 shows GYS1 knockdown in HEK293 cells using amiRNA molecule directed against human GYS1.

### DETAILED DESCRIPTION

The present disclosure provides, inter alia, isolated nucleic acid molecules, recombinant adeno-associated virus (rAAV) vectors, and rAAV viral vectors comprising at least one polynucleotide sequence encoding at least one amiRNA molecule directed against GYS1. The present disclosure also provides methods of manufacturing these isolated polynucleotides, rAAV vectors, and rAAV viral vectors, as well as their use to deliver shRNA molecules to treat or prevent LD or APBD.

The term “adeno-associated virus” or “AAV” as used herein refers to a member of the class of viruses associated with this name and belonging to the genus Dependoparvovirus, family Parvoviridae. Adeno-associated virus is a single-stranded DNA virus that grows in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3: 1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control. Multiple serotypes of this virus are known to be suitable for gene delivery; all known serotypes can infect cells from various tissue types. At least 11 sequentially numbered AAV serotypes are known in the art. Non-limiting exemplary serotypes useful in the methods disclosed herein include any of the 11 serotypes, e.g., AAV2, AAV8, AAV9, or variant serotypes, e.g., AAV-DJ and AAV PHP.B. The AAV particle comprises, consists essentially of, or consists of three major viral proteins: VP1, VP2 and VP3. In some aspects, the AAV refers to the serotype AAV1, AAV2, AAV4, AAV5, AAV6, AAV7, AAV8, AAV9, AAV10, AAV11, AAV12, AAV13, AAVPHP.B, AAVrh74 or AAVrh.10.

Exemplary adeno-associated viruses and recombinant adeno-associated viruses include, but are not limited to all serotypes (e.g., AAV1, AAV2, AAV3, AAV4, AAV5, AAV6, AAV7, AAV8, AAV9, AAV10, AAV11, AAV12, AAV13, AAVPHP.B, AAVrh74 and AAVrh.10). Exemplary adeno-associated viruses and recombinant adeno-associated viruses include, but are not limited to, self-complementary AAV (scAAV) and AAV hybrids containing the genome of one serotype and the capsid of another serotype (e.g., AAV2/5, AAV-DJ and AAV-DJ8). Exemplary adeno-associated viruses and recombinant adeno-associated viruses include, but are not limited to, rAAV-LK03, AAV-KP-1 (described in detail in Kerun et al. JCI Insight, 2019; 4(22):e131610) and AAV-NP59 (described in detail in Paulk et al. Molecular Therapy, 2018; 26(1): 289-303).

### AAV Structure and Function

AAV is a replication-deficient parvovirus, the single-stranded DNA genome of which is about 4.7 kb in length, including two 145-nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_001862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). The sequence of the AAV rh.74 genome is provided in U.S. Pat. No. 9,434,928. U.S. Pat. No. 9,434,928 also provides the sequences of the capsid proteins and a self-complementary genome. In one aspect, an AAV genome is a self-complementary genome. In some embodiments, the AAV viral vector is an AAV9 viral vector.

Cis-acting sequences directing viral DNA replication (rep), encapsidation/packaging, and host cell chromosome integration are contained within AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome.

The cap gene is expressed from the p40 promoter and encodes the three capsid proteins, VP1, VP2, and VP3. Alternative splicing and non-consensus translational start sites are responsible for the production of the three related capsid proteins. More specifically, after the single mRNA from which each of the VP1, VP2 and VP3 proteins are translated is transcribed, it can be spliced in two different manners: either a longer or shorter intron can be excised, resulting in the formation of two pools of mRNAs: a 2.3 kb- and a 2.6 kb-long mRNA pool. The longer intron is often preferred and thus the 2.3-kb-long mRNA can be called the major splice variant. This form lacks the first AUG codon, from which the synthesis of VP1 protein starts, resulting in a reduced overall level of VP1 protein synthesis. The first AUG codon that remains in the major splice variant is the initiation codon for the VP3 protein. However, upstream of that codon in the same open reading frame lies an ACG sequence (encoding threonine) which is surrounded by an optimal Kozak (translation initiation) context. This contributes to a low level of synthesis of the VP2 protein, which is actually the VP3 protein with additional N terminal residues, as is VP1, as described in Becerra S P et al., (December 1985). “Direct mapping of adeno-associated virus capsid proteins B and C: a possible ACG initiation codon”. Proceedings of the National Academy of Sciences of the United States of America. 82 (23): 7919-23, Cassinotti P et al., (November 1988). “Organization of the adeno-associated virus (AAV) capsid gene: mapping of a minor spliced mRNA coding for virus capsid protein 1”. Virology. 167 (1): 176-84, Muralidhar S et al., (January 1994). “Site-directed mutagenesis of adeno-associated virus type 2 structural protein initiation codons: effects on regulation of synthesis and biological activity”. Journal of Virology. 68 (1): 170-6, and Trempe J P, Carter B J (September 1988). “Alternate mRNA splicing is required for synthesis of adeno-associated virus VP1 capsid protein” Journal of Virology. 62 (9): 3356-63, each of which is herein incorporated by reference. A single consensus polyA site is located at map position 95 of the AAV genome. The life cycle and genetics of AAV are reviewed in Muzyczka, Current Topics in Microbiology and Immunology, 158: 97-129 (1992).

Each VP1 protein contains a VP1 portion, a VP2 portion and a VP3 portion. The VP1 portion is the N-terminal portion of the VP1 protein that is unique to the VP1 protein. The VP2 portion is the amino acid sequence present within the VP1 protein that is also found in the N-terminal portion of the VP2 protein. The VP3 portion and the VP3 protein have the same sequence. The VP3 portion is the C-terminal portion of the VP1 protein that is shared with the VP1 and VP2 proteins.

The VP3 protein can be further divided into discrete variable surface regions I-IX (VR-I-IX). Each of the variable surface regions (VRs) can comprise or contain specific amino acid sequences that either alone or in combination with the specific amino acid sequences of each of the other VRs can confer unique infection phenotypes (e.g., decreased antigenicity, improved transduction and/or tissue-specific tropism relative to other AAV serotypes) to a particular serotype as described in DiMatta et al., “Structural Insight into the Unique Properties of Adeno-Associated Virus Serotype 9” J. Virol., Vol. 86 (12): 6947-6958, June 2012, the contents of which are incorporated herein by reference.

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA to generate rAAV vectors. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8: 659-669 (1997); Kessler et al., Proc Nat Acad Sc USA, 93: 14082-14087 (1996); and Xiao et al., J Virol, 70: 8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94: 5804-5809 (1997) and Murphy et al., Proc Natl Acad Sci USA, 94: 13921-13926 (1997). Moreover, Lewis et al., J Virol, 76: 8769-8775 (2002) demonstrated that skeletal myofibers possess the necessary cellular factors for correct antibody glycosylation, folding, and secretion, indicating that muscle is capable of stable expression of secreted protein therapeutics.

Recombinant AAV (rAAV) genomes of the invention comprise, consist essentially of, or consist of a nucleic acid molecule comprising a polynucleotide sequencing encoding at least one amiRNA directed against GYS1 and one or more AAV ITRs flanking the nucleic acid molecule. Production of pseudotyped rAAV is disclosed in, for example, WO2001083692. Other types of rAAV variants, for example rAAV with capsid mutations, are also contemplated. See, e.g., Marsic et al., Molecular Therapy, 22(11): 1900-1909 (2014). The nucleotide sequences of the genomes of various AAV serotypes are known in the art. In some embodiments, the rAAV genome is an AAV9 genome.

### Isolated Nucleic Acid Molecules

The present disclosure provides isolated nucleic acid molecules comprising a polynucleotide sequence encoding at least one amiRNA directed against GYS1. In some embodiments, the amiRNA is directed against exon 8 of GYS1.

Illustrative sequences of polynucleotides encoding amiRNA sequences directed against GYS1 are set forth in Table 1. In some embodiments, the polynucleotide sequence encoding the amiRNA directed against GYS1 comprises, consists essentially of, or consists of the nucleic acid sequence set forth in SEQ ID NO: 1. In some embodiments, the polynucleotide sequence encoding the amiRNA directed against GYS1 comprises, consists essentially of, or consists of the nucleic acid sequence set forth in SEQ ID NO: 2. In some embodiments, the polynucleotide sequence encoding the amiRNA directed against GYS1 comprises, consists essentially of, or consists of the nucleic acid sequence set forth in SEQ ID NO: 3. In some embodiments, the polynucleotide sequence encoding the amiRNA directed against GYS1 comprises, consists essentially of, or consists of the nucleic acid sequence set forth in SEQ ID NO: 4. In some embodiments, the polynucleotide sequence encoding the amiRNA directed against GYS1 comprises, consists essentially of, or consists of the nucleic acid sequence set forth in SEQ ID NO: 5.

In some embodiments, the polynucleotide sequence encoding the amiRNA directed against GYS1 comprises, consists essentially of, or consists of a nucleic acid sequence that is at least 65%, 70%, 75%, 80%, 85%, 90%, 95%, 96%, 97%, 98%, 99 or 100% (or any percentage in between) identical to the nucleic acid sequence set forth in SEQ ID NO: 1. In some embodiments, the polynucleotide sequence encoding the amiRNA directed against GYS1 comprises, consists essentially of, or consists of a nucleic acid sequence that is at least 65%, 70%, 75%, 80%, 85%, 90%, 95%, 96%, 97%, 98%, 99 or 100% (or any percentage in between) identical to the nucleic acid sequence set forth in SEQ ID NO: 2. In some embodiments, the polynucleotide sequence encoding the amiRNA directed against GYS1 comprises, consists essentially of, or consists of a nucleic acid sequence that is at least 65%, 70%, 75%, 80%, 85%, 90%, 95%, 96%, 97%, 98%, 99% or 100% (or any percentage in between) identical to the nucleic acid sequence set forth in SEQ ID NO: 3. In some embodiments, the polynucleotide sequence encoding the amiRNA directed against GYS1 comprises, consists essentially of, or consists of a nucleic acid sequence that is at least 65%, 70%, 75%, 80%, 85%, 90%, 95%, 96%, 97%, 98%, 99% or 100% (or any percentage in between) identical to the nucleic acid sequence set forth in SEQ ID NO: 4. In some embodiments, the polynucleotide sequence encoding the amiRNA directed against GYS1 comprises, consists essentially of, or consists of a nucleic acid sequence that is at least 65%, 70%, 75%, 80%, 85%, 90%, 95%, 96%, 97%, 98%, 99% or 100% (or any percentage in between) identical to the nucleic acid sequence set forth in SEQ ID NO: 5.

TABLE 1
Illustrative sequences encoding amiRNAs directed against GYS1

| miRNA | Sequence |
|---|---|
| amiRNA2-33 | CCGGTAGGGCTCTGCGTTTGCTCCAGGTAGTCCGCTGCTCCCTTGG |
| | GCCTGGGCCCACTGACAGCCCTGGTGCCTCTGGCCGGCTGCACAC |
| | CTCCTGGCGGGCAGCTGTGAAAACTTCTTCACATTCAGCCTGTTC |
| | TGGCAATACCTGGGCTGAATCAGGAGAAGTTTTCACGGAGGCCTGC |
| | CCTGACTGCCCACGGTGCCGTGGCCAAAGAGGATCTAAGGGCACC |
| | GCTGAGGGCCTACCTAACCATCGTGGGGAATAAGGACAGTGTCAC |
| | CGGATC (SEQ ID NO: 1) |
| amiRNA4-33 | CCGGTAGGGCTCTGCGTTTGCTCCAGGTAGTCCGCTGCTCCCTTGG |
| | GCCTGGGCCCACTGACAGCCCTGGTGCCTCTGGCCGGCTGCACAC |
| | CTCCTGGCGGGCAGCTGTGTAAGGATTCATAAAGCTTCCTTGTTC |
| | TGGCAATACCTGAGGAAGCTAAACGAATCCTTACACGGAGGCCTGC |
| | CCTGACTGCCCACGGTGCCGTGGCCAAAGAGGATCTAAGGGCACC |
| | GCTGAGGGCCTACCTAACCATCGTGGGGAATAAGGACAGTGTCAC |
| | CCGGATC (SEQ ID NO: 2) |
| amiRNA5-33 | CCGGTAGGGCTCTGCGTTTGCTCCAGGTAGTCCGCTGCTCCCTTGG |
| | GCCTGGGCCCACTGACAGCCCTGGTGCCTCTGGCCGGCTGCACAC |
| | CTCCTGGCGGGCAGCTGTGTCATCATAGTGAAGTCTTCCTTGTTC |
| | TGGCAATACCTGAGGAAGACAACGCTATGATGACACGGAGGCCTG |
| | CCCTGACTGCCCACGGTGCCGTGGCCAAAGAGGATCTAAGGGCAC |
| | CGCTGAGGGCCTACCTAACCATCGTGGGGAATAAGGACAGTGTCA |
| | CCCGGATC (SEQ ID NO: 3) |
| amiRNA9-33 | CCGGTAGGGCTCTGCGTTTGCTCCAGGTAGTCCGCTGCTCCCTTGG |
| | GCCTGGGCCCACTGACAGCCCTGGTGCCTCTGGCCGGCTGCACAC |
| | CTCCTGGCGGGCAGCTGTGAAGGATTCATAAAGCTTCCTCTGTTC |
| | TGGCAATACCTGGAGGAAGCAATGTGAATCCTTCACGGAGGCCTGC |
| | CCTGACTGCCCACGGTGCCGTGGCCAAAGAGGATCTAAGGGCACC |
| | GCTGAGGGCCTACCTAACCATCGTGGGGAATAAGGACAGTGTCAC |
| | CCGGATC (SEQ ID NO: 4) |
| amiRNA24-33 | CCGGTAGGGCTCTGCGTTTGCTCCAGGTAGTCCGCTGCTCCCTTG |
| | GGCCTGGGCCCACTGACAGCCCTGGTGCCTCTGGCCGGCTGCACA |
| | CCTCCTGGCGGGCAGCTGTGAGTGTCGTGACAGCGAGGGCGTGTT |
| | CTGGCAATACCTGCGCCCTCGGAGCCACGACACTCACGGAGGCCT |
| | GCCCTGACTGCCCACGGTGCCGTGGCCAAAGAGGATCTAAGGGCA |
| | CCGCTGAGGGCCTACCTAACCATCGTGGGGAATAAGGACAGTGTC |
| | ACCCGGATC (SEQ ID NO: 5) |

Generally, a polynucleotide sequence encoding the amiRNA directed against GYS1 comprises provided herein comprises a the amiRNA directed against GYS1 (a mature antisense sequence) and a passenger sequence flanked by 5′ and 3′ overhangs. The sequences of the amiRNAs directed against GYS1 are shown in bold in the sequences presented in Table 1. The loop region sequences are italicized in the sequences presented in Table 1. The passenger sequences are underlined in the sequences presented in Table 1. The remaining parts of the sequences presented in Table 1 (in regular type) are the 5′ and 3′ overhangs. The loop sequence and overhangs together make up the miRNA “backbone.”

Without wishing to be bound by theory, it is believed that the passenger sequence and the amiRNA directed against GYS1 adhere to form a hairpin loop structure. The hairpin loop structures are then believed to be further processed by Dicer, an endoribonuclease which removes the loop of the hairpin, leaving a miRNA duplex. The amiRNA directed against GYS1 of the miRNA duplex is then integrated into the RNA-induced silencing complex (RISC), where it interacts with its target mRNA (e.g., GYS1 mRNA) and thus blocks translation. See Myburgh et al., Molecular Therapy—Nucleic Acids (2014) 3, e207.

Examples of the sequences of the amiRNA directed against GYS1 comprises are set forth in Table 2. In some embodiments, the amiRNA directed against GYS1 comprises, consists essentially of, or consists of the nucleic acid sequence set forth in SEQ ID NO: 35. In some embodiments, the amiRNA directed against GYS1 comprises, consists essentially of, or consists of the nucleic acid sequence set forth in SEQ ID NO: 36. In some embodiments, the amiRNA directed against GYS1 comprises, consists essentially of, or consists of the nucleic acid sequence set forth in SEQ ID NO: 37. In some embodiments, the amiRNA directed against GYS1 comprises, consists essentially of, or consists of the nucleic acid sequence set forth in SEQ ID NO: 38. In some embodiments, the amiRNA directed against GYS1 comprises, consists essentially of, or consists of the nucleic acid sequence set forth in SEQ ID NO: 39.

In some embodiments, the amiRNA directed against GYS1 comprises, consists essentially of, or consists of a nucleic acid sequence at least 65%, 70%, 75%, 80%, 85%, 90%, 95%, 96%, 97%, 98%, 99 or 100% (or any percentage in between) identical to the sequence set forth in SEQ ID NO: 35 or a complement thereof. In some embodiments, the amiRNA directed against GYS1 comprises, consists essentially of, or consists of a nucleic acid sequence at least 65%, 70%, 75%, 80%, 85%, 90%, 95%, 96%, 97%, 98%, 99 or 100% (or any percentage in between) identical to the sequence set forth in SEQ ID NO: 36 or a complement thereof. In some embodiments, the amiRNA directed against GYS1 comprises, consists essentially of, or consists of a nucleic acid sequence at least 65%, 70%, 75%, 80%, 85%, 90%, 95%, 96%, 97%, 98%, 99 or 100% (or any percentage in between) identical to the sequence set forth in SEQ ID NO: 37 or a complement thereof. In some embodiments, the amiRNA directed against GYS1 comprises, consists essentially of, or consists of a nucleic acid sequence at least 65%, 70%, 75%, 80%, 85%, 90%, 95%, 96%, 97%, 98%, 99 or 100% (or any percentage in between) identical to the sequence set forth in SEQ ID NO: 38 or a complement thereof.

In some embodiments, the amiRNA directed against GYS1 comprises, consists essentially of, or consists of a nucleic acid sequence at least 65%, 70%, 75%, 80%, 85%, 90%, 95%, 96%, 97%, 98%, 99 or 100% (or any percentage in between) identical to the sequence set forth in SEQ ID NO: 39 or a complement thereof.

TABLE 2
Examples of Sequences of amiRNAs
directed against GYSI

| miRNA | Sequence |
|---|---|
| amiRNA2-33 | AAAACTTCTTCACATTCAGCC (SEQ ID NO: 35) |
| amiRNA4-33 | TAAGGATTCATAAAGCTTCCT (SEQ ID NO: 36) |
| amiRNA5-33 | TCATCATAGTGAAGTCTTCCT (SEQ ID NO: 37) |
| amiRNA9-33 | AAGGATTCATAAAGCTTCCTC (SEQ ID NO: 38) |
| amiRNA24-33 | AGTGTCGTGACAGCGAGGGCG (SEQ ID NO: 39) |

In some embodiments, the GYS1 transcript against an amiRNA described herein is directed is a human GYS1 transcript. Illustrative human GYS1 transcripts against which the amiRNAs provided herein may be directed include those set forth in NCBI Reference Sequences NM_002103.5 (SEQ ID NO: 12) and NM_001161587.2 (SEQ ID NO: 13).

As would be appreciated by the skilled artisan, the phrase “amiRNA directed against GYS1” refers to an RNA molecule that, once produced within a cell, directs endogenous RNAi pathways (e.g. the Dicer pathway, the RNA-induced silencing complex (RISC) pathway) to initiate degradation and/or downregulation of GYS1 mRNA. The term “Gys1 targeting AAV-amiRNA” refers to an rAAV vector comprising at least one polynucleotide sequence encoding an amiRNA directed against GYS1.

In some aspects, an isolated nucleic acid molecule can comprise more than one polynucleotide sequence encoding an amiRNA directed against GYS1. Thus, in some embodiments, an isolated nucleic acid molecules comprises at least about two, or at least about three, or at least about four, or at least about five, or at least about six, or at least about seven, or at least about eight, or at least about nine, or at least about ten, or at least about 11, or at least about 12, or at least about 13, or at least about 14, or at least about 15, or at least about 16, or at least about 17, or at least about 18, or at least about 19, or at least about 20 polynucleotide sequences, each encoding an amiRNA directed against GYS1.

In aspects wherein an isolated nucleic acid molecule comprises more than one polynucleotide sequence encoding an amiRNA directed against GYS1, any number of the polynucleotide sequences may be the same sequence, and any number may be a different sequence. In a non-limiting example wherein an isolated nucleic acid molecule comprises three polynucleotide sequences each encoding an amiRNA directed against GYS1 (i.e. a first polynucleotide sequence, a second polynucleotide sequence, and a third polynucleotide sequence) all three of the polynucleotide sequences can have the same sequence. Alternatively, all three of the polynucleotide sequences can have a different sequence. Alternatively still, two of the three polynucleotide sequences can have the same sequence and the third polynucleotide sequence can have a different sequence.

rAAV Vectors

In some aspects, the isolated polynucleotides comprising at least one polynucleotide sequence encoding at least one amiRNA directed against GYS1 described herein can be a recombinant AAV (rAAV) vector.

As used herein, the term “vector” refers to a nucleic acid comprising, consisting essentially of, or consisting of an intact replicon such that the vector may be replicated when placed within a cell, for example by a process of transfection, infection, or transformation. It is understood in the art that once inside a cell, a vector may replicate as an extrachromosomal (episomal) element or may be integrated into a host cell chromosome. Vectors may include nucleic acids derived from retroviruses, adenoviruses, herpesvirus, baculoviruses, modified baculoviruses, papovaviruses, or otherwise modified naturally-occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising, consisting essentially of, or consisting of DNA condensed with cationic polymers such as heterogeneous polylysine, defined-length oligopeptides, and polyethyleneimine, in some cases contained in liposomes; and the use of ternary complexes comprising, consisting essentially of, or consisting of a virus and polylysine-DNA.

With respect to general recombinant techniques, vectors that contain both a promoter and a cloning site into which a polynucleotide can be operatively linked are well known in the art. Such vectors are capable of transcribing RNA in vitro or in vivo, and are commercially available from sources such as Agilent Technologies (Santa Clara, Calif) and Promega Biotech (Madison, Wis.). In order to optimize expression and/or in vitro transcription, it may be necessary to remove, add or alter 5′ and/or 3′ untranslated portions of cloned transgenes to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

An “rAAV vector” as used herein refers to a vector comprising, consisting essentially of, or consisting of one or more transgene and/or exogenous polynucleotide sequences and one or more AAV inverted terminal repeat sequences (ITRs). Such rAAV vectors can be replicated and packaged into infectious viral particles when present in a host cell that provides the functionality of rep and cap gene products; for example, by transfection of the host cell. In some aspects, rAAV vectors contain a promoter, at least one nucleic acid that may encode at least one protein or RNA, and/or an enhancer and/or a terminator within the flanking ITRs that is packaged into the infectious AAV particle. The encapsidated nucleic acid portion may be referred to as the rAAV vector genome. Plasmids containing rAAV vectors may also contain elements for manufacturing purposes, e.g., antibiotic resistance genes, origin of replication sequences etc., but these are not encapsidated and thus do not form part of the AAV particle.

In some aspects, an rAAV vector can comprise at least one polynucleotide sequence encoding at least one amiRNA directed against GYS1. In some aspects, an rAAV vector can comprise at least one AAV inverted terminal (ITR) sequence. In some aspects, an rAAV vector can comprise at least one promoter sequence. In some aspects, an rAAV vector can comprise at least one enhancer sequence. In some aspects, an rAAV vector can comprise at least one polyA sequence.

In some aspects, an rAAV vector can comprise a first AAV ITR sequence, a promoter sequence, at least one polynucleotide sequence encoding at least one amiRNA directed against GYS1 and a second AAV ITR sequence. In some aspects, an rAAV vector can comprise, in the 5′ to 3′ direction, a first AAV ITR sequence, a promoter sequence, at least one polynucleotide sequence encoding at least amiRNA directed against GYS1 and a second AAV ITR sequence.

In some aspects, an rAAV vector can comprise a first AAV ITR sequence, a promoter sequence, at least one polynucleotide sequence encoding at least one amiRNA directed against GYS1, a polyA sequence, and a second AAV ITR sequence. In some aspects, an rAAV vector can comprise, in the 5′ to 3′ direction, a first AAV ITR sequence, a promoter sequence, at least one polynucleotide sequence encoding at least one amiRNA directed against GYS1, a polyA sequence, and a second AAV ITR sequence.

In some aspects, an rAAV vector can comprise more than one polynucleotide sequence encoding at least one amiRNA directed against GYS1.

In some aspects, the present disclosure provides rAAV vectors comprising at least about two, or at least about three, or at least about four, or at least about five, or at least about six, or at least about seven, or at least about eight, or at least about nine, or at least about ten, or at least about 11, or at least about 12, or at least about 13, or at least about 14, or at least about 15, or at least about 16, or at least about 17, or at least about 18, or at least about 19, or at least about 20 polynucleotide sequences, each encoding at least one amiRNA directed against GYS1.

In some aspects, the present disclosure provides rAAV vectors comprising about two, or about three, or about four, or about five, or about six, or about seven, or about eight, or about nine, or about ten, or about 11, or about 12, or about 13, or about 14, or about 15, or about 16, or about 17, or about 18, or about 19, or about 20 polynucleotide sequences, each encoding at least one amiRNA directed against GYS1.

In aspects wherein an rAAV vector comprises more than one polynucleotide sequence encoding at least one amiRNA directed against GYS1, any number of the polynucleotide sequences may be the same sequence, and any number may be a different sequence. In a non-limiting example wherein an rAAV vector comprises three polynucleotide sequences encoding at least one amiRNA directed against GYS1 (i.e. a first polynucleotide sequence, a second polynucleotide sequence, and a third polynucleotide sequence), all three of the polynucleotide sequences can have the same sequence. Alternatively, all three of the polynucleotide sequences can have a different sequence. Alternatively still, two of the three polynucleotide sequences can have the same sequence and the third polynucleotide sequence can have a different sequence.

In some aspects, an rAAV vector can comprise more than one promoter sequence. In some aspects, an rAAV vector can comprise at least two promoter sequences, such that the rAAV vector comprises a first promoter sequence and an at least second promoter sequence. In some aspects, the first and the at least second promoter sequences can comprise the same sequence. In some aspects, the first and the at least second promoter sequences can comprise different sequences. In some aspects, the first and the at least second promoter sequences can be adjacent to each other. In some aspects wherein an rAAV vector also comprises a first polynucleotide sequence encoding at least one amiRNA directed against GYS1 and an at least second polynucleotide sequence encoding at least one amiRNA directed against GYS1, the first promoter can be located upstream (5′) of the first polynucleotide sequence and the at least second promoter can be located between the first polynucleotide sequence and the at least second polynucleotide sequence, such that the at least second promoter is downstream (3′) of the first polynucleotide sequence and upstream (5′) of the at least second polynucleotide sequence.

Any of the preceding rAAV vectors can further comprise at least one enhancer. The at least one enhancer can be located anywhere in the rAAV vector.

In some aspects, the at least one enhancer can be located immediately upstream (5′) of a promoter. Thus, an rAAV vector can comprise, in the 5′ to 3′ direction, a first AAV ITR sequence, an enhancer, a promoter sequence, at least one polynucleotide sequence encoding at least one amiRNA directed against GYS1 and a second AAV ITR sequence. In some aspects, the at least one enhancer can be located immediately downstream (3′) of a promoter. Thus, an rAAV vector can comprise, in the 5′ to 3′ direction, a first AAV ITR sequence, a promoter sequence, an enhancer, at least one polynucleotide sequence encoding at least one amiRNA directed against GYS1, and a second AAV ITR sequence. In some aspects, the at least one enhancer can be located immediately downstream of an at least one polynucleotide sequence encoding at least one amiRNA directed against GYS1. Thus, an rAAV vector can comprise, in the 5′ to 3′ direction, a first AAV ITR sequence, a promoter sequence, at least one polynucleotide sequence encoding at least one amiRNA directed against GYS1, an enhancer, a polyA sequence, and a second AAV ITR sequence.

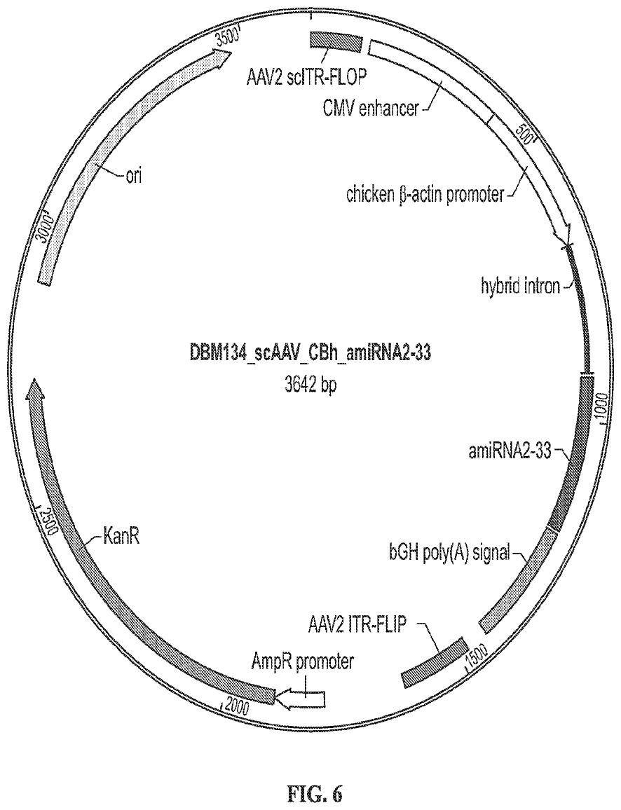

Illustrative sequences of AAV genomes described herein are set forth in Table 3. FIG. 6 shows an exemplary AAV genome comprising a first AAV2 ITR, a CMV enhancer, a chicken β, a hybrid intron, a polynucleotide sequence encoding an amiRNA directed against GYS1, a bGH poly(a) signal, a second AAV2 ITR. In some embodiments, the rAAV vector comprises the sequence set forth in any one of SEQ ID NOs: 6-10.

TABLE 3
Illustrative AAV vector sequences

| Name | Sequence |
|---|---|
| amiRN | CTGCGCGCTCGCTCGCTCACTGAGGCCGCC |
| A2-33 | GGCCCAAAGGGCCGGCGTCGGGCGACCTTT |
| AAV | GGTCGCCCGGCCTCAGTGAGCGAGCGAGCG |
| vector | CGCAGAGAGGGAGTGGAGATCTGAATTCGG |
| | TACCCGTTACATAACTTACGGTAAATGGCC |
| | CGCCTGGCTGACCGCCCAACGACCCCCGCC |
| | CATTGACGTCAATAGTAACGCCAATAGGGA |
| | CTTTCCATTGACGTCAATGGGTGGAGTATT |
| | TACGGTAAACTGCCCACTTGGCAGTACATC |
| | AAGTGTATCATATGCCAAGTACGCCCCCTA |
| | TTGACGTCAATGACGGTAAATGGCCCGCCT |
| | GGCATTGTGCCCAGTACATGACCTTATGGG |
| | ACTTTCCTACTTGGCAGTACATCTACGTAT |
| | TAGTCATCGCTATTACCATGGTCGAGGTGA |
| | GCCCCACGTTCTGCTTCACTCTCCCCATCT |
| | CCCCCCCCTCCCCACCCCCAATTTTGTATT |
| | TATTTATTTTTTAATTATTTTGTGCAGCGA |
| | TGGGGGCGGGGGGGGGGGGGGGGCGCGCGC |
| | CAGGCGGGGCGGGGCGGGGCGAGGGGCGGG |
| | GCGGGGCGAGGCGGAGAGGTGCGGCGGCAG |
| | CCAATCAGAGCGGCGCGCTCCGAAAGTTTC |
| | CTTTTATGGCGAGGCGGCGGCGGCGGCGGC |
| | CCTATAAAAAGCGAAGCGCGCGGCGGGCGG |
| | GAGTCGCTGCGCGCTGCCTTCGCCCCGTGC |
| | CCCGCTCCGCCGCCGCCTCGCGCCGCCCGC |
| | CCCGGCTCTGACTGACCGCGTTACTCCCAC |
| | AGGTGAGCGGGCGGGACGGCCCTTCTCCTC |
| | CGGGCTGTAATTAGCTGAGCAAGAGGTAAG |
| | GGTTTAAGGGATGGTTGGTTGGTGGGGTAT |
| | TAATGTTTAATTACCTGGAGCACCTGCCTG |
| | AAATCACTTTTTTTCAGGTTGGACCGGTAG |
| | GGCTCTGCGTTTGCTCCAGGTAGTCCGCTG |
| | CTCCCTTGGGCCTGGGCCCACTGACAGCCC |
| | TGGTGCCTCTGGCCGGCTGCACACCTCCTG |
| | GCGGGCAGCTGTGAAAACTTCTTCACATTC |
| | AGCCTGTTCTGGCAATACCTGGGCTGAATC |
| | AGGAGAAGTTTTCACGGAGGCCTGCCCTGA |
| | CTGCCCACGGTGCCGTGGCCAAAGAGGATC |
| | TAAGGGCACCGCTGAGGGCCTACCTAACCA |
| | TCGTGGGGAATAAGGACAGTGTCACCGGAT |
| | CCCTCGACTGTGCCTTCTAGTTGCCAGCCA |
| | TCTGTTGTTTGCCCCTCCCCCGTGCCTTCC |
| | TTGACCCTGGAAGGTGCCACTCCCACTGTC |
| | CTTTCCTAATAAAATGAGGAAATTGCATCG |
| | CATTGTCTGAGTAGGTGTCATTCTATTCTG |
| | GGGGGTGGGGTGGGGCAGGACAGCAAGGGG |
| | GAGGATTGGGAAGACAACAGCAGGCATGCT |
| | GGGGATGCGGTGGGCTCTATGGCTTCTGAG |
| | GCGGAAAGAACCAGCTTTGGACGCGTGCAT |
| | GCAGATCTAGGAACCCCTAGTGATGGAGTT |
| | GGCCACTCCCTCTCTGCGCGCTCGCTCGCT |
| | CACTGAGGCCGGGCGACCAAAGGTCGCCCG |
| | ACGCCGGCCCTTTGGGCCGGCGGCCTCAGT |
| | GAGCGAGCGAGCGCGCAGAGAGGGAGTGGC |
| | CAAGATGGAGTCAGCTGCTTACAGACAAGC |
| | TGTGACCGTCTCCGGGAGCTGCATGTGTCA |
| | GAGGTTTTCACCGTCATCACCGAAACGCGC |
| | GAGACGAAAGGGCCTCGTGATACGCCTATT |
| | TTTATAGGTTAATGTCATGATAATAATGGT |
| | TTCTTAGACGTCAGGGTTTAAACCGCGGAA |
| | CCCCTATTTGTTTATTTTTCTAAATACATT |
| | CAAATATGTATCCGCTCATGAGACAATAAC |
| | CCTGATAAATGCTTCAATAATATTGAAAAA |
| | GGAAGAGTATGAGCCATATTCAACGGGAAA |
| | CGTCTTGCTCGAGGCCGCGATTAAATTCCA |
| | ACATGGATGCTGATTTATATGGGTATAAAT |
| | GGGCTCGCGATAATGTCGGGCAATCAGGTG |
| | CGACAATCTATCGATTGTATGGGAAGCCCG |
| | ATGCGCCAGAGTTGTTTCTGAAACATGGCA |
| | AAGGTAGCGTTGCCAATGATGTTACAGATG |
| | AGATGGTCAGACTAAACTGGCTGACGGAAT |
| | TTATGCCTCTTCCGACCATCAAGCATTTTA |
| | TCCGTACTCCTGATGATGCATGGTTACTCA |
| | CCACTGCGATCCCCGGCAAAACAGCATTCC |
| | AGGTATTAGAAGAATATCCTGATTCAGGTG |
| | AAAATATTGTTGATGCGCTGGCAGTGTTCC |
| | TGCGCCGGTTGCATTCGATTCCTGTTTGTA |
| | ATTGTCCTTTTAACAGCGATCGTGTATTTC |
| | GTCTCGCTCAGGCGCAATCACGAATGAATA |
| | ACGGTTTGGTTGATGCGAGTGATTTTGATG |
| | ACGAGCGTAATGGCTGGCCTGTTGAACAAG |
| | TCTGGAAAGAAATGCATAAGCTTTTGCCAT |
| | TCTCACCGGATTCAGTCGTCACTCATGGTG |
| | ATTTCTCACTTGATAACCTTATTTTTGACG |
| | AGGGGAAATTAATAGGTTGTATTGATGTTG |
| | GACGAGTCGGAATCGCAGACCGATACCAGG |
| | ATCTTGCCATCCTATGGAACTGCCTCGGTG |
| | AGTTTTCTCCTTCATTACAGAAACGGCTTT |
| | TTCAAAAATATGGTATTGATAATCCTGATA |
| | TGAATAAATTGCAGTTTCATTTGATGCTCG |
| | ATGAGTTTTTCTAAGTTTAAACCTGTCAGA |
| | CCAAGTTTACTCATATATACTTTAGATTGA |
| | TTTAAAACTTCATTTTTAATTTAAAAGGAT |
| | CTAGGTACATGTGAAGATCCTTTTTGATAA |
| | TCTCATGACCAAAATCCCTTAACGTGAGTT |
| | TTCGTTCCACTGAGCGTCAGACCCCGGCGG |
| | CCGCTTGAGATCCTTTTTTTCTGCGCGTAA |
| | TCTGCTGCTTGCAAACAAAAAAACCACCGC |
| | TACCAGCGGTGGTTTGTTTGCCGGATCAAG |
| | AGCTACCAACTCTTTTTCCGAAGGTAACTG |
| | GCTTCAGCAGAGCGCAGATACCAAATACTG |
| | TTCTTCTAGTGTAGCCGTAGTTAGGCCACC |
| | ACTTCAAGAACTCTGTAGCACCGCCTACAT |
| | ACCTCGCTCTGCTAATCCTGTTACCAGTGG |
| | CTGCTGCCAGTGGCGATAAGTCGTGTCTTA |
| | CCGGGTTGGACTCAAGACGATAGTTACCGG |
| | ATAAGGCGCAGCGGTCGGGCTGAACGGGGG |
| | GTTCGTGCACACAGCCCAGCTTGGAGCGAA |
| | CGACCTACACCGAACTGAGATACCTACAGC |
| | GTGAGCTATGAGAAAGCGCCACGCTTCCCG |
| | AAGGGAGAAAGGCGGACAGGTATCCGGTAA |
| | GCGGCAGGGTCGGAACAGGAGAGCGCACGA |
| | GGGAGCTTCCAGGGGGAAACGCCTGGTATC |
| | TTTATAGTCCTGTCGGGTTTCGCCACCTCT |
| | GACTTGAGCGTCGATTTTTGTGATGCTCGT |
| | CAGGGGGGCGGAGCCTATGGAAAGCGGCCG |
| | CAACGCCAGCAACGCGGCCTTTTTACGGTT |
| | CCTGGCCTTTTGCTGGCCTTTTGCTCACGT |
| | GTTCTTTCCTGCGTTATCCCCTGATTCTGT |
| | GGATAACCGTATTACCGCCTTTGAGTGAGC |
| | TGATACCGCTCGCCGCAGCCGAACGAGACA |
| | GCTGAAACGTTA |
| | (SEQ ID NO: 6) |
| amiRN | CTGCGCGCTCGCTCGCTCACTGAGGCCGCC |
| A4-33 | GGCCCAAAGGGCCGGCGTCGGGCGACCTTT |
| AAV | GGTCGCCCGGCCTCAGTGAGCGAGCGAGCG |
| vector | CGCAGAGAGGGAGTGGAGATCTGAATTCGG |
| | TACCCGTTACATAACTTACGGTAAATGGCC |
| | CGCCTGGCTGACCGCCCAACGACCCCCGCC |
| | CATTGACGTCAATAGTAACGCCAATAGGGA |
| | CTTTCCATTGACGTCAATGGGTGGAGTATT |
| | TACGGTAAACTGCCCACTTGGCAGTACATC |
| | AAGTGTATCATATGCCAAGTACGCCCCCTA |
| | TTGACGTCAATGACGGTAAATGGCCCGCCT |
| | GGCATTGTGCCCAGTACATGACCTTATGGG |
| | ACTTTCCTACTTGGCAGTACATCTACGTAT |
| | TAGTCATCGCTATTACCATGGTCGAGGTGA |
| | GCCCCACGTTCTGCTTCACTCTCCCCATCT |
| | CCCCCCCCTCCCCACCCCCAATTTTGTATT |
| | TATTTATTTTTTAATTATTTTGTGCAGCGA |
| | TGGGGGCGGGGGGGGGGGGGGGGCGCGCGC |
| | CAGGCGGGGCGGGGCGGGGCGAGGGGCGGG |
| | GCGGGGCGAGGCGGAGAGGTGCGGCGGCAG |
| | CCAATCAGAGCGGCGCGCTCCGAAAGTTTC |
| | CTTTTATGGCGAGGCGGCGGCGGCGGCGGC |
| | CCTATAAAAAGCGAAGCGCGCGGCGGGCGG |
| | GAGTCGCTGCGCGCTGCCTTCGCCCCGTGC |
| | CCCGCTCCGCCGCCGCCTCGCGCCGCCCGC |
| | CCCGGCTCTGACTGACCGCGTTACTCCCAC |
| | AGGTGAGCGGGCGGGACGGCCCTTCTCCTC |
| | CGGGCTGTAATTAGCTGAGCAAGAGGTAAG |
| | GGTTTAAGGGATGGTTGGTTGGTGGGGTAT |
| | TAATGTTTAATTACCTGGAGCACCTGCCTG |
| | AAATCACTTTTTTTCAGGTTGGACCGGTAG |
| | GGCTCTGCGTTTGCTCCAGGTAGTCCGCTG |
| | CTCCCTTGGGCCTGGGCCCACTGACAGCCC |
| | TGGTGCCTCTGGCCGGCTGCACACCTCCTG |
| | GCGGGCAGCTGTGTAAGGATTCATAAAGCT |
| | TCCTTGTTCTGGCAATACCTGAGGAAGCTA |
| | AACGAATCCTTACACGGAGGCCTGCCCTGA |
| | CTGCCCACGGTGCCGTGGCCAAAGAGGATC |
| | TAAGGGCACCGCTGAGGGCCTACCTAACCA |
| | TCGTGGGGAATAAGGACAGTGTCACCCGGA |
| | TCCCTCGACTGTGCCTTCTAGTTGCCAGCC |
| | ATCTGTTGTTTGCCCCTCCCCCGTGCCTTC |
| | CTTGACCCTGGAAGGTGCCACTCCCACTGT |
| | CCTTTCCTAATAAAATGAGGAAATTGCATC |
| | GCATTGTCTGAGTAGGTGTCATTCTATTCT |
| | GGGGGGTGGGGTGGGGCAGGACAGCAAGGG |
| | GGAGGATTGGGAAGACAACAGCAGGCATGC |
| | TGGGGATGCGGTGGGCTCTATGGCTTCTGA |
| | GGCGGAAAGAACCAGCTTTGGACGCGTGCA |
| | TGCAGATCTAGGAACCCCTAGTGATGGAGT |
| | TGGCCACTCCCTCTCTGCGCGCTCGCTCGC |
| | TCACTGAGGCCGGGCGACCAAAGGTCGCCC |
| | GACGCCGGCCCTTTGGGCCGGCGGCCTCAG |
| | TGAGCGAGCGAGCGCGCAGAGAGGGAGTGG |
| | CCAAGATGGAGTCAGCTGCTTACAGACAAG |
| | CTGTGACCGTCTCCGGGAGCTGCATGTGTC |
| | AGAGGTTTTCACCGTCATCACCGAAACGCG |
| | CGAGACGAAAGGGCCTCGTGATACGCCTAT |
| | TTTTATAGGTTAATGTCATGATAATAATGG |
| | TTTCTTAGACGTCAGGGTTTAAACCGCGGA |
| | ACCCCTATTTGTTTATTTTTCTAAATACAT |
| | TCAAATATGTATCCGCTCATGAGACAATAA |
| | CCCTGATAAATGCTTCAATAATATTGAAAA |
| | AGGAAGAGTATGAGCCATATTCAACGGGAA |
| | ACGTCTTGCTCGAGGCCGCGATTAAATTCC |
| | AACATGGATGCTGATTTATATGGGTATAAA |
| | TGGGCTCGCGATAATGTCGGGCAATCAGGT |
| | GCGACAATCTATCGATTGTATGGGAAGCCC |
| | GATGCGCCAGAGTTGTTTCTGAAACATGGC |
| | AAAGGTAGCGTTGCCAATGATGTTACAGAT |
| | GAGATGGTCAGACTAAACTGGCTGACGGAA |
| | TTTATGCCTCTTCCGACCATCAAGCATTTT |
| | ATCCGTACTCCTGATGATGCATGGTTACTC |
| | ACCACTGCGATCCCCGGCAAAACAGCATTC |
| | CAGGTATTAGAAGAATATCCTGATTCAGGT |
| | GAAAATATTGTTGATGCGCTGGCAGTGTTC |
| | CTGCGCCGGTTGCATTCGATTCCTGTTTGT |
| | AATTGTCCTTTTAACAGCGATCGTGTATTT |
| | CGTCTCGCTCAGGCGCAATCACGAATGAAT |
| | AACGGTTTGGTTGATGCGAGTGATTTTGAT |
| | GACGAGCGTAATGGCTGGCCTGTTGAACAA |
| | GTCTGGAAAGAAATGCATAAGCTTTTGCCA |
| | TTCTCACCGGATTCAGTCGTCACTCATGGT |
| | GATTTCTCACTTGATAACCTTATTTTTGAC |
| | GAGGGGAAATTAATAGGTTGTATTGATGTT |
| | GGACGAGTCGGAATCGCAGACCGATACCAG |
| | GATCTTGCCATCCTATGGAACTGCCTCGGT |
| | GAGTTTTCTCCTTCATTACAGAAACGGCTT |
| | TTTCAAAAATATGGTATTGATAATCCTGAT |
| | ATGAATAAATTGCAGTTTCATTTGATGCTC |
| | GATGAGTTTTTCTAAGTTTAAACCTGTCAG |
| | ACCAAGTTTACTCATATATACTTTAGATTG |
| | ATTTAAAACTTCATTTTTAATTTAAAAGGA |
| | TCTAGGTACATGTGAAGATCCTTTTTGATA |
| | ATCTCATGACCAAAATCCCTTAACGTGAGT |
| | TTTCGTTCCACTGAGCGTCAGACCCCGGCG |
| | GCCGCTTGAGATCCTTTTTTTCTGCGCGTA |
| | ATCTGCTGCTTGCAAACAAAAAAACCACCG |
| | CTACCAGCGGTGGTTTGTTTGCCGGATCAA |
| | GAGCTACCAACTCTTTTTCCGAAGGTAACT |
| | GGCTTCAGCAGAGCGCAGATACCAAATACT |
| | GTTCTTCTAGTGTAGCCGTAGTTAGGCCAC |
| | CACTTCAAGAACTCTGTAGCACCGCCTACA |
| | TACCTCGCTCTGCTAATCCTGTTACCAGTG |
| | GCTGCTGCCAGTGGCGATAAGTCGTGTCTT |
| | ACCGGGTTGGACTCAAGACGATAGTTACCG |
| | GATAAGGCGCAGCGGTCGGGCTGAACGGGG |
| | GGTTCGTGCACACAGCCCAGCTTGGAGCGA |
| | ACGACCTACACCGAACTGAGATACCTACAG |
| | CGTGAGCTATGAGAAAGCGCCACGCTTCCC |
| | GAAGGGAGAAAGGCGGACAGGTATCCGGTA |
| | AGCGGCAGGGTCGGAACAGGAGAGCGCACG |
| | AGGGAGCTTCCAGGGGGAAACGCCTGGTAT |
| | CTTTATAGTCCTGTCGGGTTTCGCCACCTC |
| | TGACTTGAGCGTCGATTTTTGTGATGCTCG |
| | TCAGGGGGGCGGAGCCTATGGAAAGCGGCC |
| | GCAACGCCAGCAACGCGGCCTTTTTACGGT |
| | TCCTGGCCTTTTGCTGGCCTTTTGCTCACG |
| | TGTTCTTTCCTGCGTTATCCCCTGATTCTG |
| | TGGATAACCGTATTACCGCCTTTGAGTGAG |
| | CTGATACCGCTCGCCGCAGCCGAACGAGAC |
| | AGCTGAAACGTTA |
| | (SEQ ID NO: 7) |
| amiRN | CTGCGCGCTCGCTCGCTCACTGAGGCCGCC |
| A5-33 | GGCCCAAAGGGCCGGCGTCGGGCGACCTTT |
| AAV | GGTCGCCCGGCCTCAGTGAGCGAGCGAGCG |
| vector | CGCAGAGAGGGAGTGGAGATCTGAATTCGG |
| | TACCCGTTACATAACTTACGGTAAATGGCC |
| | CGCCTGGCTGACCGCCCAACGACCCCCGCC |
| | CATTGACGTCAATAGTAACGCCAATAGGGA |
| | CTTTCCATTGACGTCAATGGGTGGAGTATT |
| | TACGGTAAACTGCCCACTTGGCAGTACATC |
| | AAGTGTATCATATGCCAAGTACGCCCCCTA |
| | TTGACGTCAATGACGGTAAATGGCCCGCCT |
| | GGCATTGTGCCCAGTACATGACCTTATGGG |
| | ACTTTCCTACTTGGCAGTACATCTACGTAT |
| | TAGTCATCGCTATTACCATGGTCGAGGTGA |
| | GCCCCACGTTCTGCTTCACTCTCCCCATCT |
| | CCCCCCCCTCCCCACCCCCAATTTTGTATT |
| | TATTTATTTTTTAATTATTTTGTGCAGCGA |
| | TGGGGGCGGGGGGGGGGGGGGGGCGCGCGC |
| | CAGGCGGGGCGGGGCGGGGCGAGGGGCGGG |
| | GCGGGGCGAGGCGGAGAGGTGCGGCGGCAG |
| | CCAATCAGAGCGGCGCGCTCCGAAAGTTTC |
| | CTTTTATGGCGAGGCGGCGGCGGCGGCGGC |
| | CCTATAAAAAGCGAAGCGCGCGGCGGGCGG |
| | GAGTCGCTGCGCGCTGCCTTCGCCCCGTGC |
| | CCCGCTCCGCCGCCGCCTCGCGCCGCCCGC |
| | CCCGGCTCTGACTGACCGCGTTACTCCCAC |
| | AGGTGAGCGGGCGGGACGGCCCTTCTCCTC |
| | CGGGCTGTAATTAGCTGAGCAAGAGGTAAG |
| | GGTTTAAGGGATGGTTGGTTGGTGGGGTAT |
| | TAATGTTTAATTACCTGGAGCACCTGCCTG |
| | AAATCACTTTTTTTCAGGTTGGACCGGTAG |
| | GGCTCTGCGTTTGCTCCAGGTAGTCCGCTG |
| | CTCCCTTGGGCCTGGGCCCACTGACAGCCC |
| | TGGTGCCTCTGGCCGGCTGCACACCTCCTG |
| | GCGGGCAGCTGTGTCATCATAGTGAAGTCT |
| | TCCTTGTTCTGGCAATACCTGAGGAAGACA |
| | ACGCTATGATGACACGGAGGCCTGCCCTGA |
| | CTGCCCACGGTGCCGTGGCCAAAGAGGATC |
| | TAAGGGCACCGCTGAGGGCCTACCTAACCA |
| | TCGTGGGGAATAAGGACAGTGTCACCCGGA |
| | TCCCTCGACTGTGCCTTCTAGTTGCCAGCC |
| | ATCTGTTGTTTGCCCCTCCCCCGTGCCTTC |
| | CTTGACCCTGGAAGGTGCCACTCCCACTGT |
| | CCTTTCCTAATAAAATGAGGAAATTGCATC |
| | GCATTGTCTGAGTAGGTGTCATTCTATTCT |
| | GGGGGGTGGGGTGGGGCAGGACAGCAAGGG |
| | GGAGGATTGGGAAGACAACAGCAGGCATGC |
| | TGGGGATGCGGTGGGCTCTATGGCTTCTGA |
| | GGCGGAAAGAACCAGCTTTGGACGCGTGCA |
| | TGCAGATCTAGGAACCCCTAGTGATGGAGT |
| | TGGCCACTCCCTCTCTGCGCGCTCGCTCGC |
| | TCACTGAGGCCGGGCGACCAAAGGTCGCCC |
| | GACGCCGGCCCTTTGGGCCGGCGGCCTCAG |
| | TGAGCGAGCGAGCGCGCAGAGAGGGAGTGG |
| | CCAAGATGGAGTCAGCTGCTTACAGACAAG |
| | CTGTGACCGTCTCCGGGAGCTGCATGTGTC |
| | AGAGGTTTTCACCGTCATCACCGAAACGCG |
| | CGAGACGAAAGGGCCTCGTGATACGCCTAT |
| | TTTTATAGGTTAATGTCATGATAATAATGG |
| | TTTCTTAGACGTCAGGGTTTAAACCGCGGA |
| | ACCCCTATTTGTTTATTTTTCTAAATACAT |
| | TCAAATATGTATCCGCTCATGAGACAATAA |
| | CCCTGATAAATGCTTCAATAATATTGAAAA |
| | AGGAAGAGTATGAGCCATATTCAACGGGAA |
| | ACGTCTTGCTCGAGGCCGCGATTAAATTCC |
| | AACATGGATGCTGATTTATATGGGTATAAA |
| | TGGGCTCGCGATAATGTCGGGCAATCAGGT |
| | GCGACAATCTATCGATTGTATGGGAAGCCC |
| | GATGCGCCAGAGTTGTTTCTGAAACATGGC |
| | AAAGGTAGCGTTGCCAATGATGTTACAGAT |
| | GAGATGGTCAGACTAAACTGGCTGACGGAA |
| | TTTATGCCTCTTCCGACCATCAAGCATTTT |
| | ATCCGTACTCCTGATGATGCATGGTTACTC |
| | ACCACTGCGATCCCCGGCAAAACAGCATTC |
| | CAGGTATTAGAAGAATATCCTGATTCAGGT |
| | GAAAATATTGTTGATGCGCTGGCAGTGTTC |
| | CTGCGCCGGTTGCATTCGATTCCTGTTTGT |
| | AATTGTCCTTTTAACAGCGATCGTGTATTT |
| | CGTCTCGCTCAGGCGCAATCACGAATGAAT |
| | AACGGTTTGGTTGATGCGAGTGATTTTGAT |
| | GACGAGCGTAATGGCTGGCCTGTTGAACAA |
| | GTCTGGAAAGAAATGCATAAGCTTTTGCCA |
| | TTCTCACCGGATTCAGTCGTCACTCATGGT |
| | GATTTCTCACTTGATAACCTTATTTTTGAC |
| | GAGGGGAAATTAATAGGTTGTATTGATGTT |
| | GGACGAGTCGGAATCGCAGACCGATACCAG |
| | GATCTTGCCATCCTATGGAACTGCCTCGGT |
| | GAGTTTTCTCCTTCATTACAGAAACGGCTT |
| | TTTCAAAAATATGGTATTGATAATCCTGAT |
| | ATGAATAAATTGCAGTTTCATTTGATGCTC |
| | GATGAGTTTTTCTAAGTTTAAACCTGTCAG |
| | ACCAAGTTTACTCATATATACTTTAGATTG |
| | ATTTAAAACTTCATTTTTAATTTAAAAGGA |
| | TCTAGGTACATGTGAAGATCCTTTTTGATA |
| | ATCTCATGACCAAAATCCCTTAACGTGAGT |
| | TTTCGTTCCACTGAGCGTCAGACCCCGGCG |
| | GCCGCTTGAGATCCTTTTTTTCTGCGCGTA |
| | ATCTGCTGCTTGCAAACAAAAAAACCACCG |
| | CTACCAGCGGTGGTTTGTTTGCCGGATCAA |
| | GAGCTACCAACTCTTTTTCCGAAGGTAACT |
| | GGCTTCAGCAGAGCGCAGATACCAAATACT |
| | GTTCTTCTAGTGTAGCCGTAGTTAGGCCAC |
| | CACTTCAAGAACTCTGTAGCACCGCCTACA |
| | TACCTCGCTCTGCTAATCCTGTTACCAGTG |
| | GCTGCTGCCAGTGGCGATAAGTCGTGTCTT |
| | ACCGGGTTGGACTCAAGACGATAGTTACCG |
| | GATAAGGCGCAGCGGTCGGGCTGAACGGGG |
| | GGTTCGTGCACACAGCCCAGCTTGGAGCGA |
| | ACGACCTACACCGAACTGAGATACCTACAG |
| | CGTGAGCTATGAGAAAGCGCCACGCTTCCC |
| | GAAGGGAGAAAGGCGGACAGGTATCCGGTA |
| | AGCGGCAGGGTCGGAACAGGAGAGCGCACG |
| | AGGGAGCTTCCAGGGGGAAACGCCTGGTAT |
| | CTTTATAGTCCTGTCGGGTTTCGCCACCTC |
| | TGACTTGAGCGTCGATTTTTGTGATGCTCG |
| | TCAGGGGGGCGGAGCCTATGGAAAGCGGCC |
| | GCAACGCCAGCAACGCGGCCTTTTTACGGT |
| | TCCTGGCCTTTTGCTGGCCTTTTGCTCACG |
| | TGTTCTTTCCTGCGTTATCCCCTGATTCTG |
| | TGGATAACCGTATTACCGCCTTTGAGTGAG |
| | CTGATACCGCTCGCCGCAGCCGAACGAGAC |
| | AGCTGAAACGTTA |
| | (SEQ ID NO: 8) |
| amiRN | CTGCGCGCTCGCTCGCTCACTGAGGCCGCC |
| A9-33 | GGCCCAAAGGGCCGGCGTCGGGCGACCTTT |
| AAV | GGTCGCCCGGCCTCAGTGAGCGAGCGAGCG |
| vector | CGCAGAGAGGGAGTGGAGATCTGAATTCGG |
| | TACCCGTTACATAACTTACGGTAAATGGCC |
| | CGCCTGGCTGACCGCCCAACGACCCCCGCC |
| | CATTGACGTCAATAGTAACGCCAATAGGGA |
| | CTTTCCATTGACGTCAATGGGTGGAGTATT |
| | TACGGTAAACTGCCCACTTGGCAGTACATC |
| | AAGTGTATCATATGCCAAGTACGCCCCCTA |
| | TTGACGTCAATGACGGTAAATGGCCCGCCT |
| | GGCATTGTGCCCAGTACATGACCTTATGGG |
| | ACTTTCCTACTTGGCAGTACATCTACGTAT |
| | TAGTCATCGCTATTACCATGGTCGAGGTGA |
| | GCCCCACGTTCTGCTTCACTCTCCCCATCT |
| | CCCCCCCCTCCCCACCCCCAATTTTGTATT |
| | TATTTATTTTTTAATTATTTTGTGCAGCGA |
| | TGGGGGCGGGGGGGGGGGGGGGGCGCGCGC |
| | CAGGCGGGGCGGGGCGGGGCGAGGGGCGGG |
| | GGGGGCGAGGCGGAGAGGTGCGGCGGCAGC |
| | CAATCAGAGCGGCGCGCTCCGAAAGTTTCC |
| | TTTTATGGCGAGGCGGCGGCGGCGGCGGCC |
| | CTATAAAAAGCGAAGCGCGCGGCGGGCGGG |
| | AGTCGCTGCGCGCTGCCTTCGCCCCGTGCC |
| | CCGCTCCGCCGCCGCCTCGCGCCGCCCGCC |
| | CCGGCTCTGACTGACCGCGTTACTCCCACA |
| | GGTGAGCGGGCGGGACGGCCCTTCTCCTCC |
| | GGGCTGTAATTAGCTGAGCAAGAGGTAAGG |
| | GTTTAAGGGATGGTTGGTTGGTGGGGTATT |
| | AATGTTTAATTACCTGGAGCACCTGCCTGA |
| | AATCACTTTTTTTCAGGTTGGACCGGTAGG |
| | GCTCTGCGTTTGCTCCAGGTAGTCCGCTGC |
| | TCCCTTGGGCCTGGGCCCACTGACAGCCCT |
| | GGTGCCTCTGGCCGGCTGCACACCTCCTGG |
| | CGGGCAGCTGTGAAGGATTCATAAAGCTTC |
| | CTCTGTTCTGGCAATACCTGGAGGAAGCAA |
| | TGTGAATCCTTCACGGAGGCCTGCCCTGAC |
| | TGCCCACGGTGCCGTGGCCAAAGAGGATCT |
| | AAGGGCACCGCTGAGGGCCTACCTAACCAT |
| | CGTGGGGAATAAGGACAGTGTCACCCGGAT |
| | CCCTCGACTGTGCCTTCTAGTTGCCAGCCA |
| | TCTGTTGTTTGCCCCTCCCCCGTGCCTTCC |
| | TTGACCCTGGAAGGTGCCACTCCCACTGTC |
| | CTTTCCTAATAAAATGAGGAAATTGCATCG |
| | CATTGTCTGAGTAGGTGTCATTCTATTCTG |
| | GGGGGTGGGGTGGGGCAGGACAGCAAGGGG |
| | GAGGATTGGGAAGACAACAGCAGGCATGCT |
| | GGGGATGCGGTGGGCTCTATGGCTTCTGAG |
| | GCGGAAAGAACCAGCTTTGGACGCGTGCAT |
| | GCAGATCTAGGAACCCCTAGTGATGGAGTT |
| | GGCCACTCCCTCTCTGCGCGCTCGCTCGCT |
| | CACTGAGGCCGGGCGACCAAAGGTCGCCCG |
| | ACGCCGGCCCTTTGGGCCGGCGGCCTCAGT |
| | GAGCGAGCGAGCGCGCAGAGAGGGAGTGGC |
| | CAAGATGGAGTCAGCTGCTTACAGACAAGC |
| | TGTGACCGTCTCCGGGAGCTGCATGTGTCA |
| | GAGGTTTTCACCGTCATCACCGAAACGCGC |
| | GAGACGAAAGGGCCTCGTGATACGCCTATT |
| | TTTATAGGTTAATGTCATGATAATAATGGT |
| | TTCTTAGACGTCAGGGTTTAAACCGCGGAA |
| | CCCCTATTTGTTTATTTTTCTAAATACATT |
| | CAAATATGTATCCGCTCATGAGACAATAAC |
| | CCTGATAAATGCTTCAATAATATTGAAAAA |
| | GGAAGAGTATGAGCCATATTCAACGGGAAA |
| | CGTCTTGCTCGAGGCCGCGATTAAATTCCA |
| | ACATGGATGCTGATTTATATGGGTATAAAT |
| | GGGCTCGCGATAATGTCGGGCAATCAGGTG |
| | CGACAATCTATCGATTGTATGGGAAGCCCG |
| | ATGCGCCAGAGTTGTTTCTGAAACATGGCA |
| | AAGGTAGCGTTGCCAATGATGTTACAGATG |
| | AGATGGTCAGACTAAACTGGCTGACGGAAT |
| | TTATGCCTCTTCCGACCATCAAGCATTTTA |
| | TCCGTACTCCTGATGATGCATGGTTACTCA |
| | CCACTGCGATCCCCGGCAAAACAGCATTCC |
| | AGGTATTAGAAGAATATCCTGATTCAGGTG |
| | AAAATATTGTTGATGCGCTGGCAGTGTTCC |
| | TGCGCCGGTTGCATTCGATTCCTGTTTGTA |
| | ATTGTCCTTTTAACAGCGATCGTGTATTTC |
| | GTCTCGCTCAGGCGCAATCACGAATGAATA |
| | ACGGTTTGGTTGATGCGAGTGATTTTGATG |
| | ACGAGCGTAATGGCTGGCCTGTTGAACAAG |
| | TCTGGAAAGAAATGCATAAGCTTTTGCCAT |
| | TCTCACCGGATTCAGTCGTCACTCATGGTG |
| | ATTTCTCACTTGATAACCTTATTTTTGACG |
| | AGGGGAAATTAATAGGTTGTATTGATGTTG |
| | GACGAGTCGGAATCGCAGACCGATACCAGG |
| | ATCTTGCCATCCTATGGAACTGCCTCGGTG |
| | AGTTTTCTCCTTCATTACAGAAACGGCTTT |
| | TTCAAAAATATGGTATTGATAATCCTGATA |
| | TGAATAAATTGCAGTTTCATTTGATGCTCG |
| | ATGAGTTTTTCTAAGTTTAAACCTGTCAGA |
| | CCAAGTTTACTCATATATACTTTAGATTGA |
| | TTTAAAACTTCATTTTTAATTTAAAAGGAT |
| | CTAGGTACATGTGAAGATCCTTTTTGATAA |
| | TCTCATGACCAAAATCCCTTAACGTGAGTT |
| | TTCGTTCCACTGAGCGTCAGACCCCGGCGG |
| | CCGCTTGAGATCCTTTTTTTCTGCGCGTAA |
| | TCTGCTGCTTGCAAACAAAAAAACCACCGC |
| | TACCAGCGGTGGTTTGTTTGCCGGATCAAG |
| | AGCTACCAACTCTTTTTCCGAAGGTAACTG |
| | GCTTCAGCAGAGCGCAGATACCAAATACTG |
| | TTCTTCTAGTGTAGCCGTAGTTAGGCCACC |
| | ACTTCAAGAACTCTGTAGCACCGCCTACAT |
| | ACCTCGCTCTGCTAATCCTGTTACCAGTGG |
| | CTGCTGCCAGTGGCGATAAGTCGTGTCTTA |
| | CCGGGTTGGACTCAAGACGATAGTTACCGG |
| | ATAAGGCGCAGCGGTCGGGCTGAACGGGGG |
| | GTTCGTGCACACAGCCCAGCTTGGAGCGAA |
| | CGACCTACACCGAACTGAGATACCTACAGC |
| | GTGAGCTATGAGAAAGCGCCACGCTTCCCG |
| | AAGGGAGAAAGGCGGACAGGTATCCGGTAA |
| | GCGGCAGGGTCGGAACAGGAGAGCGCACGA |
| | GGGAGCTTCCAGGGGGAAACGCCTGGTATC |
| | TTTATAGTCCTGTCGGGTTTCGCCACCTCT |
| | GACTTGAGCGTCGATTTTTGTGATGCTCGT |
| | CAGGGGGGCGGAGCCTATGGAAAGCGGCCG |
| | CAACGCCAGCAACGCGGCCTTTTTACGGTT |
| | CCTGGCCTTTTGCTGGCCTTTTGCTCACGT |
| | GTTCTTTCCTGCGTTATCCCCTGATTCTGT |
| | GGATAACCGTATTACCGCCTTTGAGTGAGC |
| | TGATACCGCTCGCCGCAGCCGAACGAGACA |
| | GCTGAAACGTTA |
| | (SEQ ID NO: 9) |
| amiRN | CTGCGCGCTCGCTCGCTCACTGAGGCCGCC |
| A24-33 | GGCCCAAAGGGCCGGCGTCGGGCGACCTTT |
| AAV | GGTCGCCCGGCCTCAGTGAGCGAGCGAGCG |
| vector | CGCAGAGAGGGAGTGGAGATCTGAATTCGG |
| | TACCCGTTACATAACTTACGGTAAATGGCC |
| | CGCCTGGCTGACCGCCCAACGACCCCCGCC |
| | CATTGACGTCAATAGTAACGCCAATAGGGA |
| | CTTTCCATTGACGTCAATGGGTGGAGTATT |
| | TACGGTAAACTGCCCACTTGGCAGTACATC |
| | AAGTGTATCATATGCCAAGTACGCCCCCTA |
| | TTGACGTCAATGACGGTAAATGGCCCGCCT |
| | GGCATTGTGCCCAGTACATGACCTTATGGG |
| | ACTTTCCTACTTGGCAGTACATCTACGTAT |
| | TAGTCATCGCTATTACCATGGTCGAGGTGA |
| | GCCCCACGTTCTGCTTCACTCTCCCCATCT |
| | CCCCCCCCTCCCCACCCCCAATTTTGTATT |
| | TATTTATTTTTTAATTATTTTGTGCAGCGA |
| | TGGGGGCGGGGGGGGGGGGGGGGCGCGCGC |
| | CAGGCGGGGCGGGGCGGGGCGAGGGGCGGG |
| | GCGGGGCGAGGCGGAGAGGTGCGGCGGCAG |
| | CCAATCAGAGCGGCGCGCTCCGAAAGTTTC |
| | CTTTTATGGCGAGGCGGCGGCGGCGGCGGC |
| | CCTATAAAAAGCGAAGCGCGCGGCGGGCGG |
| | GAGTCGCTGCGCGCTGCCTTCGCCCCGTGC |
| | CCCGCTCCGCCGCCGCCTCGCGCCGCCCGC |
| | CCCGGCTCTGACTGACCGCGTTACTCCCAC |
| | AGGTGAGCGGGCGGGACGGCCCTTCTCCTC |
| | CGGGCTGTAATTAGCTGAGCAAGAGGTAAG |
| | GGTTTAAGGGATGGTTGGTTGGTGGGGTAT |
| | TAATGTTTAATTACCTGGAGCACCTGCCTG |
| | AAATCACTTTTTTTCAGGTTGGACCGGTAG |
| | GGCTCTGCGTTTGCTCCAGGTAGTCCGCTG |
| | CTCCCTTGGGCCTGGGCCCACTGACAGCCC |
| | TGGTGCCTCTGGCCGGCTGCACACCTCCTG |
| | GCGGGCAGCTGTGAGTGTCGTGACAGCGAG |
| | GGCGTGTTCTGGCAATACCTGCGCCCTCGG |
| | AGCCACGACACTCACGGAGGCCTGCCCTGA |
| | CTGCCCACGGTGCCGTGGCCAAAGAGGATC |
| | TAAGGGCACCGCTGAGGGCCTACCTAACCA |
| | TCGTGGGGAATAAGGACAGTGTCACCCGGA |
| | TCCCTCGACTGTGCCTTCTAGTTGCCAGCC |
| | ATCTGTTGTTTGCCCCTCCCCCGTGCCTTC |
| | CTTGACCCTGGAAGGTGCCACTCCCACTGT |
| | CCTTTCCTAATAAAATGAGGAAATTGCATC |
| | GCATTGTCTGAGTAGGTGTCATTCTATTCT |
| | GGGGGGTGGGGTGGGGCAGGACAGCAAGGG |
| | GGAGGATTGGGAAGACAACAGCAGGCATGC |
| | TGGGGATGCGGTGGGCTCTATGGCTTCTGA |
| | GGCGGAAAGAACCAGCTTTGGACGCGTGCA |
| | TGCAGATCTAGGAACCCCTAGTGATGGAGT |
| | TGGCCACTCCCTCTCTGCGCGCTCGCTCGC |
| | TCACTGAGGCCGGGCGACCAAAGGTCGCCC |
| | GACGCCGGCCCTTTGGGCCGGCGGCCTCAG |
| | TGAGCGAGCGAGCGCGCAGAGAGGGAGTGG |
| | CCAAGATGGAGTCAGCTGCTTACAGACAAG |
| | CTGTGACCGTCTCCGGGAGCTGCATGTGTC |
| | AGAGGTTTTCACCGTCATCACCGAAACGCG |
| | CGAGACGAAAGGGCCTCGTGATACGCCTAT |
| | TTTTATAGGTTAATGTCATGATAATAATGG |
| | TTTCTTAGACGTCAGGGTTTAAACCGCGGA |
| | ACCCCTATTTGTTTATTTTTCTAAATACAT |
| | TCAAATATGTATCCGCTCATGAGACAATAA |
| | CCCTGATAAATGCTTCAATAATATTGAAAA |
| | AGGAAGAGTATGAGCCATATTCAACGGGAA |
| | ACGTCTTGCTCGAGGCCGCGATTAAATTCC |
| | AACATGGATGCTGATTTATATGGGTATAAA |
| | TGGGCTCGCGATAATGTCGGGCAATCAGGT |
| | GCGACAATCTATCGATTGTATGGGAAGCCC |
| | GATGCGCCAGAGTTGTTTCTGAAACATGGC |
| | AAAGGTAGCGTTGCCAATGATGTTACAGAT |
| | GAGATGGTCAGACTAAACTGGCTGACGGAA |
| | TTTATGCCTCTTCCGACCATCAAGCATTTT |
| | ATCCGTACTCCTGATGATGCATGGTTACTC |
| | ACCACTGCGATCCCCGGCAAAACAGCATTC |
| | CAGGTATTAGAAGAATATCCTGATTCAGGT |
| | GAAAATATTGTTGATGCGCTGGCAGTGTTC |
| | CTGCGCCGGTTGCATTCGATTCCTGTTTGT |
| | AATTGTCCTTTTAACAGCGATCGTGTATTT |
| | CGTCTCGCTCAGGCGCAATCACGAATGAAT |
| | AACGGTTTGGTTGATGCGAGTGATTTTGAT |
| | GACGAGCGTAATGGCTGGCCTGTTGAACAA |
| | GTCTGGAAAGAAATGCATAAGCTTTTGCCA |
| | TTCTCACCGGATTCAGTCGTCACTCATGGT |
| | GATTTCTCACTTGATAACCTTATTTTTGAC |
| | GAGGGGAAATTAATAGGTTGTATTGATGTT |
| | GGACGAGTCGGAATCGCAGACCGATACCAG |
| | GATCTTGCCATCCTATGGAACTGCCTCGGT |
| | GAGTTTTCTCCTTCATTACAGAAACGGCTT |
| | TTTCAAAAATATGGTATTGATAATCCTGAT |
| | ATGAATAAATTGCAGTTTCATTTGATGCTC |
| | GATGAGTTTTTCTAAGTTTAAACCTGTCAG |
| | ACCAAGTTTACTCATATATACTTTAGATTG |
| | ATTTAAAACTTCATTTTTAATTTAAAAGGA |
| | TCTAGGTACATGTGAAGATCCTTTTTGATA |
| | ATCTCATGACCAAAATCCCTTAACGTGAGT |
| | TTTCGTTCCACTGAGCGTCAGACCCCGGCG |
| | GCCGCTTGAGATCCTTTTTTTCTGCGCGTA |
| | ATCTGCTGCTTGCAAACAAAAAAACCACCG |
| | CTACCAGCGGTGGTTTGTTTGCCGGATCAA |
| | GAGCTACCAACTCTTTTTCCGAAGGTAACT |
| | GGCTTCAGCAGAGCGCAGATACCAAATACT |
| | GTTCTTCTAGTGTAGCCGTAGTTAGGCCAC |
| | CACTTCAAGAACTCTGTAGCACCGCCTACA |
| | TACCTCGCTCTGCTAATCCTGTTACCAGTG |
| | GCTGCTGCCAGTGGCGATAAGTCGTGTCTT |
| | ACCGGGTTGGACTCAAGACGATAGTTACCG |
| | GATAAGGCGCAGCGGTCGGGCTGAACGGGG |
| | GGTTCGTGCACACAGCCCAGCTTGGAGCGA |
| | ACGACCTACACCGAACTGAGATACCTACAG |
| | CGTGAGCTATGAGAAAGCGCCACGCTTCCC |
| | GAAGGGAGAAAGGCGGACAGGTATCCGGTA |
| | AGCGGCAGGGTCGGAACAGGAGAGCGCACG |
| | AGGGAGCTTCCAGGGGGAAACGCCTGGTAT |
| | CTTTATAGTCCTGTCGGGTTTCGCCACCTC |
| | TGACTTGAGCGTCGATTTTTGTGATGCTCG |
| | TCAGGGGGGCGGAGCCTATGGAAAGCGGCC |
| | GCAACGCCAGCAACGCGGCCTTTTTACGGT |
| | TCCTGGCCTTTTGCTGGCCTTTTGCTCACG |
| | TGTTCTTTCCTGCGTTATCCCCTGATTCTG |
| | TGGATAACCGTATTACCGCCTTTGAGTGAG |
| | CTGATACCGCTCGCCGCAGCCGAACGAGAC |
| | AGCTGAAACGTTA |
| | (SEQ ID NO: 10) |

### AAV ITR Sequences

In some aspects, an AAV ITR sequence can comprise any AAV ITR sequence known in the art. In some aspects, an AAV ITR sequence can be an AAV1 ITR sequence, an AAV2 ITR sequence, an AAV4 ITR sequence, an AAV5 ITR sequence, an AAV6 ITR sequence, an AAV7 ITR sequence, an AAV8 ITR sequence, an AAV9 ITR sequence, an AAV10 ITR sequence, an AAV11 ITR sequence, an AAV12 ITR sequence, an AAV13 ITR sequence, an AAVrh74 ITR sequence or an AAVrh.10 ITR sequence.

Thus, in some aspects, an AAV ITR sequence can comprise, consist essentially of, or consist of an AAV1 ITR sequence, an AAV2 ITR sequence, an AAV4 ITR sequence, an AAV5 ITR sequence, an AAV6 ITR sequence, an AAV7 ITR sequence, an AAV8 ITR sequence, an AAV9 ITR sequence, an AAV10 ITR sequence, an AAV11 ITR sequence, an AAV12 ITR sequence, an AAV13 ITR sequence, an AAVrh74 ITR sequence, or an AAVrh.10 ITR sequence.

In some aspects, an rAAV vector of the present disclosure can comprise, consist essentially of, or consist of AAV2 ITR sequences. In some aspects, an rAAV vector of the present disclosure can comprise, consist essentially of, or consist of AAV2 ITR sequences or a modified AAV2 ITR sequence.

In some aspects, an AAV ITR sequence can comprise, consist essentially of, or consist of a nucleic acid sequence at least 65%, 70%, 75%, 80%, 85%, 90%, 95%, 96%, 97%, 98%, 99 or 100% (or any percentage in between) identical to any one of the nucleic acid sequences put forth in any one of SEQ ID NOs: 14-22, or complement thereof.

In some aspects, a first AAV ITR sequence can comprise, consist essentially of, or consist of a nucleic acid sequence at least 65%, 70%, 75%, 80%, 85%, 90%, 95%, 96%, 97%, 98%, 99 or 100% (or any percentage in between) identical to the nucleic acid sequence set forth in SEQ ID NO: 14, or complement thereof.

In some aspects, a second AAV ITR sequence can comprise, consist essentially of, or consist of a nucleic acid sequence at least 65%, 70%, 75%, 80%, 85%, 90%, 95%, 96%, 97%, 98%, 99 or 100% (or any percentage in between) identical to the nucleic acid sequence set forth in SEQ ID NO: 15, or complement thereof.

### Promoter Sequence and Enhancers

The term “promoter” and “promoter sequence” as used herein means a control sequence that is a region of a polynucleotide sequence at which the initiation and rate of transcription of a coding sequence, such as a gene or a transgene or an shRNA sequence, are controlled. Promoters may be constitutive, inducible, repressible, or tissue-specific, for example. Promoters may contain genetic elements at which regulatory proteins and molecules such as RNA polymerase and transcription factors may bind. Non-limiting exemplary promoters include Rous sarcoma virus (RSV) LTR promoter (optionally with the RSV enhancer), a cytomegalovirus (CMV) promoter, an SV40 promoter, a dihydrofolate reductase promoter, a β-actin promoter, a phosphoglycerol kinase (PGK) promoter, a U6 promoter, an H1 promoter, a ubiquitous chicken β-actin hybrid (CBh) promoter, a small nuclear RNA (U1a or U1b) promoter, an MeCP2 promoter, an MeP418 promoter, an MeP426 promoter, a minimal MeCP2 promoter, a VMD2 promoter, an mRho promoter, or an EF1 promoter.

Additional non-limiting exemplary promoters provided herein include, but are not limited to EFla, Ubc, human β-actin, CAG, TRE, Ac5, Polyhedrin, CaMKIIa, Gall, TEF1, GDS, ADH1, Ubi, and α-1-antitrypsin (hAAT). It is known in the art that the nucleotide sequences of such promoters may be modified in order to increase or decrease the efficiency of mRNA transcription. See, e.g., Gao et al. (2018) Mol. Ther.: Nucleic Acids 12:135-145 (modifying TATA box of 7SK, U6 and H1 promoters to abolish RNA polymerase III transcription and stimulate RNA polymerase II-dependent mRNA transcription). Synthetically-derived promoters may be used for ubiquitous or tissue specific expression. Further, virus-derived promoters, some of which are noted above, may be useful in the methods disclosed herein, e.g., CMV, HIV, adenovirus, and AAV promoters. In some aspects, the promoter is used together with at least one enhancer to increase the transcription efficiency. Non-limiting examples of enhancers include an interstitial retinoid-binding protein (IRBP) enhancer, an RSV enhancer or a CMV enhancer.

In some aspects, a promoter sequence can comprise, consist essentially of, or consist of a Rous sarcoma virus (RSV) LTR promoter sequence (optionally with the RSV enhancer), a cytomegalovirus (CMV) promoter sequence, an SV40 promoter sequence, a dihydrofolate reductase promoter sequence, a β-actin promoter sequence, a phosphoglycerol kinase (PGK) promoter sequence, a U6 promoter sequence, an H1 promoter sequence, a ubiquitous chicken β-actin hybrid (CBh) promoter sequence, a small nuclear RNA (U1a or U1b) promoter sequence, an MeCP2 promoter sequence, an MeP418 promoter sequence, an MeP426 promoter sequence, a meP229 promoter sequence, a minimal MeCP2 promoter sequence, a VMD2 promoter sequence, an mRho promoter sequence, an EFI promoter sequence, an EFla promoter sequence, a Ubc promoter sequence, a human β-actin promoter sequence, a CAG promoter sequence, a TRE promoter sequence, an Ac5 promoter sequence, a Polyhedrin promoter sequence, a CaMKIIa promoter sequence, a Gall promoter sequence, a TEF1 promoter sequence, a GDS promoter sequence, an ADH1 promoter sequence, a Ubi promoter sequence or an α-1-antitrypsin (hAAT) promoter sequence.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) or synthetic techniques such that transcription of that gene is directed by the linked enhancer/promoter.

Non-limiting examples of linked enhancer/promoter for use in the methods, compositions and constructs provided herein include a PDE promoter plus IRBP enhancer or a CMV enhancer plus U1a promoter. It is understood in the art that enhancers can operate from a distance and irrespective of their orientation relative to the location of an endogenous or heterologous promoter. It is thus further understood that an enhancer operating at a distance from a promoter is thus “operably linked” to that promoter irrespective of its location in the vector or its orientation relative to the location of the promoter.

As used throughout the disclosure, the term “operably linked” refers to the expression of a polynucleotide sequence (i.e. a polynucleotide sequence encoding at least one amiRNA directed against GYS1) that is under the control of a promoter with which it is spatially connected. A promoter can be positioned 5′ (upstream) or 3′ (downstream) of a polynucleotide sequence under its control. A promoter can be positioned 5′(upstream) of a polynucleotide sequence under its control. The distance between a promoter and a polynucleotide sequence under its control can be approximately the same as the distance between that promoter and the polynucleotide sequence it controls in the gene from which the promoter is derived. Variation in the distance between a promoter and a polynucleotide sequence can be accommodated without loss of promoter function.

In some aspects, a promoter sequence can comprise, consist essentially of, or consist of a JeT promoter sequence. A JeT promoter sequence can comprise, consist essentially of, or consist of a nucleic acid sequence at least 65%, 70%, 75%, 80%, 85%, 90%, 95%, 96%, 97%, 98%, 99 or 100% (or any percentage in between) identical to SEQ ID NO: 23, or complement thereof.

In some aspects, a promoter sequence can comprise, consist essentially of, or consist of a MeP229 promoter sequence. A meP229 promoter sequence can comprise, consist essentially of, or consist of a nucleic acid sequence at least 65%, 70%, 75%, 80%, 85%, 90%, 95%, 96%, 97%, 98%, 99 or 100% (or any percentage in between) identical to SEQ ID NO: 24, or a complement thereof.

In some aspects, a promoter sequence can comprise, consist essentially of, or consist of a MeP426 promoter sequence. A meP426 promoter sequence can comprise, consist essentially of, or consist of a nucleic acid sequence at least 65%, 70%, 75%, 80%, 85%, 90%, 95%, 96%, 97%, 98%, 99 or 100% (or any percentage in between) identical to SEQ ID NO: 25, or a complement thereof.

In some aspects, a promoter sequence can comprise, consist essentially of, or consist of a CBh promoter sequence. A CBh promoter sequence can comprise, consist essentially of, or consist of a nucleic acid sequence at least 65%, 70%, 75%, 80%, 85%, 90%, 95%, 96%, 97%, 98%, 99 or 100% (or any percentage in between) identical to SEQ ID NO: 26, or a complement thereof.

In some aspects, a promoter sequence can comprise, consist essentially of, or consist of a U6 promoter sequence. A U6 promoter sequence can comprise, consist essentially of, or consist of a nucleic acid sequence at least 65%, 70%, 75%, 80%, 85%, 90%, 95%, 96%, 97%, 98%, 99 or 100% (or any percentage in between) identical to SEQ ID NO: 27, or a complement thereof.

In some aspects, bacterial plasmids of the present disclosure can comprise a prokaryotic promoter. A prokaryotic promoter can comprise, consist essentially of, or consist of a nucleic acid sequence at least 65%, 70%, 75%, 80%, 85%, 90%, 95%, 96%, 97%, 98%, 99 or 100% (or any percentage in between) identical to SEQ ID NO: 28, or a complement thereof.

### PolyA Sequences

In some aspects, a polyadenylation (polyA) sequence can comprise any polyA sequence known in the art. Non-limiting examples of polyA sequences include, but are not limited to, an MeCP2 polyA sequence, a retinol dehydrogenase 1 (RDH1) polyA sequence, a bovine growth hormone (BGH) polyA sequence, an SV40 polyA sequence, a SPA49 polyA sequence, a sNRP-TK65 polyA sequence, a sNRP polyA sequence, or a TK65 polyA sequence.

Thus, a polyA sequence can comprise, consist essentially of, or consist of an MeCP2 polyA sequence, a retinol dehydrogenase 1 (RDH1) polyA sequence, a bovine growth hormone (BGH) polyA sequence, an SV40 polyA sequence, a SPA49 polyA sequence, a sNRP-TK65 polyA sequence, a sNRP polyA sequence, or a TK65 polyA sequence.

In some aspects, a polyA sequence can comprise, consist essentially of, or consist of an SV40 pA sequence. In some aspects, an SV40 pA sequence can comprise, consist essentially of, or consist of a nucleic acid sequence at least 65%, 70%, 75%, 80%, 85%, 90%, 95%, 96%, 97%, 98%, 99 or 100% (or any percentage in between) identical to the sequence put forth in SEQ ID NOs: 29, or a complement thereof.

In some aspects, a polyA sequence can comprise, consist essentially of, or consist of a BGHpA sequence. In some aspects, an BGHpA sequence can comprise, consist essentially of, or consist of a nucleic acid sequence at least 65%, 70%, 75%, 80%, 85%, 90%, 95%, 96%, 97%, 98%, 99 or 100% (or any percentage in between) identical to the sequence put forth in SEQ ID NO: 30, or complement thereof. In some aspects, an BGHpA sequence can comprise, consist essentially of, or consist of a nucleic acid sequence at least 65%, 70%, 75%, 80%, 85%, 90%, 95%, 96%, 97%, 98%, 99 or 100% (or any percentage in between) identical to the sequence put forth in SEQ ID NO: 31, or complement thereof.

In some aspects, the rAAV vectors of the present disclosure can be contained within a bacterial plasmid to allow for propagation of the rAAV vector in vitro. Thus, the present disclosure provides bacterial plasmids comprising any of the rAAV vectors described herein.

A bacterial plasmid, an rAAV vector, or an rAAV viral vector can further comprise an origin of replication sequence. In some aspects, an origin of replication sequence can comprise, consist essentially of, or consist of any origin of replication sequence known in the art. The origin of replication sequence can be a bacterial origin of replication sequence, thereby allowing the rAAV vector comprising said bacterial origin of replication sequence to be produced, propagated and maintained in bacteria, using methods standard in the art. In some embodiments, the origin of replication comprises, essentially consists of, or consists of the sequences set forth in SEQ ID NO: 34.

A bacterial plasmid, an rAAV vector, or an rAAV viral vector can further comprise an antibiotic resistance gene. In some aspects, an antibiotic resistance gene can comprise, consist essentially of, or consist of any antibiotic resistance genes known in the art. Examples of antibiotic resistance genes known in the art include, but are not limited to kanamycin resistance genes, spectinomycin resistance genes, streptomycin resistance genes, ampicillin resistance genes, carbenicillin resistance genes, bleomycin resistance genes, erythromycin resistance genes, polymyxin B resistance genes, tetracycline resistance genes and chloramphenicol resistance genes. In some embodiments, a kanamycin resistance gene comprises, essentially consists of, or consists of the sequence set forth in SEQ ID NO: 33. In some embodiments, the kanamycin resistance gene is operatively linked to a promoter. In some embodiments, the kanamycin resistance gene is operatively linked to an ampicillin resistance gene promoter (AmpR promoter). In some embodiments, the AmpR promoter comprises, essentially consists of, or consists of the sequence set forth in SEQ ID NO: 32.

A bacterial plasmid, an rAAV vector, or an rAAV viral vector can further comprise a prokaryotic promoter.

### AAV Viral Vectors

A “viral vector” is defined as a recombinantly produced virus or viral particle that contains a polynucleotide to be delivered into a host cell, either in vivo, ex vivo or in vitro. Examples of viral vectors include retroviral vectors, AAV vectors, lentiviral vectors, adenovirus vectors, alphavirus vectors and the like. Alphavirus vectors, such as Semliki Forest virus-based vectors and Sindbis virus-based vectors, have also been developed for use in gene therapy and immunotherapy. See, e.g., Schlesinger and Dubensky (1999) Curr. Opin. Biotechnol. 5:434-439 and Ying, et al. (1999) Nat. Med. 5(7):823-827.

An “AAV virion” or “AAV viral particle” or “AAV viral vector” or “rAAV viral vector” or “AAV vector particle” or “AAV particle” refers to a viral particle composed of at least one AAV capsid protein and an encapsidated polynucleotide rAAV vector. Thus, production of an rAAV viral vector necessarily includes production of an rAAV vector, as such a vector is contained within an rAAV vector.

As used herein, the term “viral capsid” or “capsid” refers to the proteinaceous shell or coat of a viral particle. Capsids function to encapsidate, protect, transport, and release into the host cell a viral genome. Capsids are generally comprised of oligomeric structural subunits of protein (“capsid proteins”). As used herein, the term “encapsidated” means enclosed within a viral capsid. The viral capsid of AAV is composed of a mixture of three viral capsid proteins: VP1, VP2, and VP3. The mixture of VP1, VP2 and VP3 contains 60 monomers that are arranged in a T=1 icosahedral symmetry in a ratio of 1:1:10 (VP1:VP2:VP3) or 1:1:20 (VP1:VP2:VP3) as described in Sonntag F et al., (June 2010). “A viral assembly factor promotes AAV2 capsid formation in the nucleolus”. Proceedings of the National Academy of Sciences of the United States of America. 107 (22): 10220-5, and Rabinowitz J E, Samulski R J (December 2000). “Building a better vector: the manipulation of AAV virions”. Virology. 278 (2): 301-8, each of which is incorporated herein by reference in its entirety.

The present disclosure provides an rAAV viral vector comprising: a) any of the rAAV vectors described herein, or a complement thereof; and b) an AAV capsid protein.

An AAV capsid protein can be any AAV capsid protein known in the art. An AAV capsid protein can be an AAV1 capsid protein, an AAV2 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV11 capsid protein, an AAV12 capsid protein, an AAV13 capsid protein, an AAVPHP.B capsid protein, an AAVrh74 capsid protein or an AAVrh.10 capsid protein. In some embodiments, the AAV capsid of an rAAV vector described herein is an AAV9 capsid.

### Compositions and Pharmaceutical Compositions

The present disclosure also provides compositions comprising any of the isolated polynucleotides, rAAV vectors, and/or rAAV viral vectors described herein. In some aspects, the compositions can be pharmaceutical compositions. Accordingly, the present disclosure provides pharmaceutical compositions comprising any of the isolated polynucleotides, rAAV vectors, and/or rAAV viral vectors described herein and optionally a pharmaceutically acceptable carrier.

The pharmaceutical composition, as described herein, may be formulated by any methods known or developed in the art of pharmacology, which include but are not limited to contacting the active ingredients (e.g., viral particles or recombinant vectors) with an excipient and/or additive and/or other accessory ingredient, dividing or packaging the product to a dose unit. The viral particles of this disclosure may be formulated with desirable features, e.g., increased stability, increased cell transfection, sustained or delayed release, biodistributions or tropisms, modulated or enhanced translation of encoded protein in vivo, and the release profile of encoded protein in vivo.

As such, the pharmaceutical composition may further comprise saline, lipidoids, liposomes, lipid nanoparticles, polymers, lipoplexes, core-shell nanoparticles, peptides, proteins, cells transfected with viral vectors (e.g., for transplantation into a subject), nanoparticle mimics or combinations thereof. In some aspects, the pharmaceutical composition is formulated as a nanoparticle. In some aspects, the nanoparticle is a self-assembled nucleic acid nanoparticle.

A pharmaceutical composition in accordance with the present disclosure may be prepared, packaged, and/or sold in bulk, as a single unit dose, and/or as a plurality of single unit doses. The amount of the active ingredient is generally equal to the dosage of the active ingredient which would be administered to a subject and/or a convenient fraction of such a dosage such as, for example, one-half or one-third of such a dosage. The formulations of the invention can include one or more excipients and/or additives, each in an amount that together increases the stability of the viral vector, increases cell transfection or transduction by the viral vector, increases the expression of viral vector encoded protein, and/or alters the release profile of viral vector encoded proteins. In some aspects, the pharmaceutical composition comprises an excipient and/or additive. Non limiting examples of excipients and/or additives include solvents, dispersion media, diluents, or other liquid vehicles, dispersion or suspension aids, surface active agents, isotonic agents, thickening or emulsifying agents, preservatives, or combination thereof.

In some aspects, the pharmaceutical composition comprises a cryoprotectant. The term “cryoprotectant” refers to an agent capable of reducing or eliminating damage to a substance during freezing. Non-limiting examples of cryoprotectants include sucrose, trehalose, lactose, glycerol, dextrose, raffinose and/or mannitol.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

In some aspects, a pharmaceutical composition of the present disclosure can comprise phosphate-buffered saline, D-sorbitol, sodium chloride, pluronic F-68 or any combination thereof.

In some aspects, a pharmaceutical composition can comprise sodium chloride, wherein the sodium chloride is present at a concentration of about 100 mM to about 500 mM, or about 200 mM to about 400 mM, or about 300 mM to about 400 mM. In some aspects, the sodium chloride can be present at a concentration of about 350 mM.

In some aspects, a pharmaceutical composition can comprise D-sorbitol, wherein the D-sorbitol is present at a concentration of about 1% to about 10%, or about 2.5% to about 7.5%. In some aspects, the D-sorbitol can be present at a concentration of about 5%.

In some aspects, a pharmaceutical composition can comprise pluronic F-68, wherein the pluronic F-68 is present at a concentration of about 0.00001% to about 0.01%, or about 0.0005% to about 0.005%. In some aspects, the pluronic F-68 can be present at a concentration of about 0.001%.

Thus, the present disclosure provides a pharmaceutical composition comprising an rAAV vector and/or rAAV viral vector of the present disclosure in a phosphate-buffered saline solution, wherein the pharmaceutical composition further comprises sodium chloride at a concentration of 350 mM, D-sorbitol at a concentration of 5% and pluronic F-68 at a concentration of 0.001%.

Thus, the present disclosure provides a pharmaceutical composition comprising an rAAV vector and/or rAAV viral vector of the present disclosure, wherein the pharmaceutical composition further comprises sodium chloride at a concentration of 350 mM, D-sorbitol at a concentration of 5% and pluronic F-68 at a concentration of 0.001%.

Thus, the present disclosure provides a pharmaceutical composition comprising an rAAV vector and/or rAAV viral vector of the present disclosure in a phosphate-buffered saline solution, wherein the pharmaceutical composition further comprises sodium chloride at a concentration of 350 mM, D-sorbitol at a concentration of 5%.

Thus, the present disclosure provides a pharmaceutical composition comprising an rAAV vector and/or rAAV viral vector of the present disclosure, wherein the pharmaceutical composition further comprises sodium chloride at a concentration of 350 mM, D-sorbitol at a concentration of 5%.

### Methods of Using the Compositions of the Disclosure

The present disclosure provides the use of a disclosed composition or pharmaceutical composition for the treatment of a disease or disorder in a cell, tissue, organ, animal, or subject, as known in the art or as described herein, using the disclosed compositions and pharmaceutical compositions, e.g., administering or contacting the cell, tissue, organ, animal, or subject with a therapeutic effective amount of the composition or pharmaceutical composition. In one aspect, the subject is a mammal. Preferably, the subject is human. The terms “subject” and “patient” are used interchangeably herein.

This disclosure provides methods of preventing or treating a disorder, comprising, consisting essentially of, or consisting of administering to a subject a therapeutically effective amount of any one of the isolated nucleic acid molecules, rAAV vectors, rAAV viral vectors, compositions and/or pharmaceutical compositions disclosed herein.

In one aspect, provided herein is a method of treating a glycogen storage disease in a subject, the method comprising administering to the subject and effective amount of an isolated nucleic acid, an rAAV vector, an rAAV viral vector, or a composition described herein. In some embodiments, the subject suffers from a disorder which is associated with dysregulation of glycogen synthase.

In some embodiments, the subject suffers from a disorder that is associated with the accumulation of normally branched glycogen in lysosomes. Accumulation of normally branched glycogen may occur, for example, due to acid maltase deficiency.

In some embodiments, the subject suffers from a disorder that is associated with overextended glycogen branches. Overextended glycogen branches may manifest as polyglucosan bodies (PBs). In some embodiments, the subject is suffering from neuroinflammation and/or neurodegeneration. In some embodiments, the subject suffers from a disorder, which is associated with decreased levels and/or functionality of glycogen branching enzyme (GBE). Such decreased levels or function of GBE may be caused, for example, by mutations in the GBE1 gene. In some embodiments, the subject suffers from a disease associated with mutations in the EPM2A and/or EPM2B genes.

In some embodiments, a subject treated in accordance with the methods described herein has one or more mutations in the GBE1 gene. In some embodiments, a subject treated in accordance with the methods described herein has one or more mutations in the EPM2A gene. In some embodiments, a subject treated in accordance with the methods described herein has one or more mutations in the EPM2B gene. One, two, or three of GBE1, EMP2A and EPM2B may be mutated in a subject.

In some embodiments, the disease is adult polyglucosan body disease (APBD). In some embodiments, the disease is Lafora disease (LD). In some embodiments, the disease is Pompe disease.

Accordingly, the present disclosure provides methods of treating LD in a subject, comprising, consisting essentially of, or consisting of administering to the subject a therapeutically effective amount of any one of the isolated nucleic acid molecules, rAAV vectors, rAAV viral vectors, compositions and/or pharmaceutical compositions disclosed herein. The present disclosure provides the use of any one of the isolated nucleic acid molecules, rAAV vectors, rAAV viral vectors, compositions and/or pharmaceutical compositions disclosed herein for the manufacture of a medicament for the treatment or prevention of LD. The present disclosure provides any one of the isolated nucleic acid molecules, rAAV vectors, rAAV viral vectors, compositions and/or pharmaceutical compositions disclosed herein for use in treating LD.

Furthermore, the present disclosure provides methods of treating APBD in a subject, comprising, consisting essentially of, or consisting of administering to the subject a therapeutically effective amount of any one of the isolated nucleic acid molecules, rAAV vectors, rAAV viral vectors, compositions and/or pharmaceutical compositions disclosed herein.

The present disclosure also provides the use of any one of the isolated nucleic acid molecules, rAAV vectors, rAAV viral vectors, compositions and/or pharmaceutical compositions disclosed herein for the manufacture of a medicament for the treatment of APBD. The present disclosure provides any one of the isolated nucleic acid molecules, rAAV vectors, rAAV viral vectors, compositions and/or pharmaceutical compositions disclosed herein for use in treating APBD.

In some aspects, a subject can be less than 0.5 years of age, or less than 1 year of age, or less than 1.5 years of age, or less than 2 years of age, or at less than 2.5 years of age, or less than 3 years of age, or less than 3.5 years of age, or less than 3.5 years of age, or less than 4 years of age, or less than 4.5 years of age, or less than 5 years of age, or less than 5.5 years of age, or less than 6 years of age, or less than 6.5 years of age, or less than 7 years of age, or less than 7.5 years of age, or less than 8 years of age, or less than 8.5 years of age, or less than 9 years of age, or less than 9.5 years of age, or less than 10 years of age. In some aspects the subject can be less than 11 years of age, less than 12 years of age, less than 13 years of age, less than 14 years of age, less than 15 years of age, less than 20 years of age, less than 30 years of age, less than 40 years of age, less than 50 years of age, less than 60 years of age, less than 70 years of age, less than 80 years of age, less than 90 years of age, less than 100 years of age, less than 110 years of age, or less than 120 years of age. In some aspects, a subject can be less than 0.5 years of age. In some aspects, a subject can be less than 4 years of age. In some aspects, a subject can be less than 10 years of age.

The methods of treatment disclosed herein may be combined with appropriate diagnostic techniques to identify and select patients for the therapy or prevention.

The disclosure provides methods of increasing the level of one or more proteins in a host cell, comprising contacting the host cell with any one of the rAAV viral vectors disclosed herein, wherein the rAAV viral vectors comprises any one of the rAAV vectors disclosed herein. In some aspects, the host cell is in vitro, in vivo, or ex vivo. In some aspects, the host cell is derived from a subject, e.g., a subject suffering from LD or from APDB.

In some aspects, administration of an isolated nucleic acid, an rAAV vector or an rAAV viral vector herein results in a decrease in GYS1 protein expression in the host cell. The level of GYS1 may be decreased to level of about 1×10⁻⁷ ng, about 3×10⁻⁷ ng, about 5×10⁻⁷ ng, about 7×10⁻⁷ ng, about 9×10⁻⁷ ng, about 1×10⁻⁶ ng, about 2×10⁻⁶ ng, about 3×10⁻⁶ ng, about 4×10⁻⁶ ng, about 6×10⁻⁶ ng, about 7×10⁻⁶ ng, about 8×10⁻⁶ ng, about 9×10⁻⁶ ng, about 10×10⁻⁶ ng, about 12×10⁻⁶ ng, about 14×10⁻⁶ ng, about 16×10⁻⁶ ng, about 18×10⁻⁶ ng, about 20×10⁻⁶ ng, about 25×10⁻⁶ ng, about 30×10⁻⁶ ng, about 35×10⁻⁶ ng, about 40×10⁻⁶ ng, about 45×10⁻⁶ ng, about 50×10⁻⁶ ng, about 55×10⁻⁶ ng, about 60×10⁻⁶ ng, about 65×10⁻⁶ ng, about 70×10⁻⁶ ng, about 75×10⁻⁶ ng, about 80×10⁻⁶ ng, about 85×10⁻⁶ ng, about 90×10⁻⁶ ng, about 95×10⁻⁶ ng, about 10×10⁻⁵ ng, about 20×10⁻⁵ ng, about 30×10⁻⁵ ng, about 40×10⁻⁵ ng, about 50×10⁻⁵ ng, about 60×10⁻⁵ ng, about 70×10⁻⁵ ng, about 80×10⁻⁵ ng, or about 90×10⁻⁵ ng in the host cell.

In some embodiments, the levels of GYS1 protein may be decreased by at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 65%, at least about 70%, or at least about 75%, or at least about 80%, or at least about 90%, or at least about 95%, or at least about 100%, or at least about 200%, or at least about 300%, or at least about 400%, or at least about 500%, or at least about 600%, or at least about 700%, or at least about 800%, or at least about 900%, or at least about 1,000%.

In some embodiments, the levels of GYS1 protein may be decreased at least about 1.25 fold, or at least bout 1.5 fold, or at least about 1.25 fold, or at least about 2 fold, or at least about 2.25 fold, or at least about 2.5 fold, or at least about 2.75 fold, or at least about 3 fold, or at least about 4 fold, or at least about 5 fold, or at least about 6 fold, or at least about 7 fold, or at least about 8 fold, or at least about 9 fold, or at least about 10 fold.

The disclosure provides methods of decreasing the level of an RNA molecule in a host cell, comprising contacting the host cell with any one of the rAAV viral vectors disclosed herein, wherein the rAAV viral vectors comprises any one of the rAAV vectors disclosed herein. In some aspects the RNA molecule is an mRNA molecule or a non-coding RNA molecule. In some aspects, the host cell is in vitro, in vivo, or ex vivo. In some aspects, the host cell is derived from a subject, e.g., a subject suffering from LD or from APBD.

In some aspects, administration of an isolated nucleic acid, an rAAV vector or an rAAV viral vector herein results in a decrease in GYS1 mRNA in the host cell. The level of the mRNA molecule may be decreased to level of about 1×10⁻⁷ ng, about 3×10⁻⁷ ng, about 5×10⁻⁷ ng, about 7×10⁻⁷ ng, about 9×10⁻⁷ ng, about 1×10⁻⁶ ng, about 2×10⁻⁶ ng, about 3×10⁻⁶ ng, about 4×10⁻⁶ ng, about 6×10⁻⁶ ng, about 7×10⁻⁶ ng, about 8×10⁻⁶ ng, about 9×10⁻⁶ ng, about 10×10⁻⁶ ng, about 12×10⁻⁶ ng, about 14×10⁻⁶ ng, about 16×10⁻⁶ ng, about 18×10⁻⁶ ng, about 20×10⁻⁶ ng, about 25×10⁻⁶ ng, about 30×10⁻⁶ ng, about 35×10⁻⁶ ng, about 40×10⁻⁶ ng, about 45×10⁻⁶ ng, about 50×10⁻⁶ ng, about 55×10⁻⁶ ng, about 60×10⁻⁶ ng, about 65×10⁻⁶ ng, about 70×10⁻⁶ ng, about 75×10⁻⁶ ng, about 80×10⁻⁶ ng, about 85×10⁻⁶ ng, about 90×10⁻⁶ ng, about 95×10⁻⁶ ng, about 10×10⁻⁵ ng, about 20×10⁻⁵ ng, about 30×10⁻⁵ ng, about 40×10⁻⁵ ng, about 50×10⁻⁵ ng, about 60×10⁻⁵ ng, about 70×10⁻⁵ ng, about 80×10⁻⁵ ng, or about 90×10⁻⁵ ng in the host cell.

In some embodiments, the level of GYS1 mRNA is decreased by at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 65%, at least about 70%, or at least about 75%, or at least about 80%, or at least about 90%, or at least about 95%, or at least about 100%, or at least about 200%, or at least about 300%, or at least about 400%, or at least about 500%, or at least about 600%, or at least about 700%, or at least about 800%, or at least about 900%, or at least about 1,000%.

In some embodiments, the level of GYS1 mRNA is decreased at least about 1.25 fold, or at least bout 1.5 fold, or at least about 1.25 fold, or at least about 2 fold, or at least about 2.25 fold, or at least about 2.5 fold, or at least about 2.75 fold, or at least about 3 fold, or at least about 4 fold, or at least about 5 fold, or at least about 6 fold, or at least about 7 fold, or at least about 8 fold, or at least about 9 fold, or at least about 10 fold.

The disclosure provides methods of introducing a polynucleotide sequence of interest (e.g. a polynucleotide sequence encoding at least one amiRNA directed against GYS1) to a cell in a subject comprising contacting the cell with an effective amount of any one of the rAAV viral vectors disclosed herein, wherein the rAAV viral vectors contain any one of the rAAV vectors disclosed herein, comprising the polynucleotide sequence of interest.

In some aspects of the methods of the present disclosure, a subject can also be administered a prophylactic immunosuppressant treatment regimen in addition to being administered an rAAV vector or rAAV viral vector of the present disclosure. In some aspects, an immunosuppressant treatment regimen can comprise administering at least one immunosuppressive therapeutic. Non limiting examples of immunosuppressive therapeutics include, but are not limited to, Sirolimus (rapamycin), acetaminophen, diphenhydramine, IV methylprednisolone, prednisone, or any combination thereof.

An immunosuppressive therapeutic can be administered prior to the day of administration of the rAAV vector and/or rAAV viral vector, on the same day as the administration of the rAAV vector and/or rAAV viral vector, or any day following the administration of the rAAV vector and/or rAAV viral vector.

A “subject” of diagnosis or treatment is a cell or an animal such as a mammal, or a human. A subject is not limited to a specific species and includes non-human animals subject to diagnosis or treatment and those subject to infections or animal models, including, without limitation, simian, murine, rat, canine, or leporid species, as well as other livestock, sport animals, or pets. In some aspects, the subject is a human.

As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. The ameliorating may be compared to another subject (e.g., a subject suffering from LD or APBD not being administered a composition, an isolated nucleic acid molecule, an rAAV vector, or rAAV viral vector described herein) or to the subject being treated prior to the first administration of the composition, isolated nucleic acid molecule, rAAV vector, or rAAV viral vector described herein.

In some embodiments, the symptom being ameliorated by administration of a composition, an isolated nucleic acid molecule, an rAAV vector, or rAAV viral vector described herein is the accumulation of polyglucosan bodies (PBs), for example, accumulation of PBs in the glia, neurones, and/or axons. PBs may be detected using any suitable method known in the art or described herein, including, for example, immunohistochemistry.

In some embodiments, the symptom being ameliorated by administration of a composition, an isolated nucleic acid molecule, an rAAV vector, or rAAV viral vector described herein is a sensory deficit, upper and/or lower motor neuron dysfunction, ataxia, and/or loss of urinary bladder control.

As used herein the term “effective amount” intends to mean a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of gene therapy, the effective amount can be the amount sufficient to result in normal or improved function of a gene that is deficient in a subject. In some embodiments, the effective amount of an isolated polynucleotide, an rAAV vector, an rAAV viral vector or a composition provided herein is the amount sufficient to result in expression of an amiRNA directed against GYS1 in the subject. In some embodiments, the effective amount of an isolated polynucleotide, an rAAV vector, an rAAV viral vector or a composition provided herein is the amount sufficient to result in expression of an amiRNA directed against GYS1 in the subject such that GBE activity is downregulated. The skilled artisan will be able to determine appropriate amounts depending on these and other factors.

In some aspects, the effective amount will depend on the size and nature of the application in question. It will also depend on the nature and sensitivity of the target subject and the methods in use. The skilled artisan will be able to determine the effective amount based on these and other considerations. The effective amount may comprise, consist essentially of, or consist of one or more administrations of a composition depending on the embodiment.

As used herein, the term “administer” or “administration” intends to mean delivery of a substance to a subject such as an animal or human. Administration can be effected in one dose, continuously or intermittently throughout the course of treatment. Methods of determining the most effective means and dosage of administration are known to those of skill in the art and will vary with the composition used for therapy, the purpose of the therapy, as well as the age, health or gender of the subject being treated. Single or multiple administrations can be carried out with the dose level and pattern being selected by the treating physician or in the case of pets and other animals, treating veterinarian.

Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. It is noted that dosage may be impacted by the route of administration. Suitable dosage formulations and methods of administering the agents are known in the art. Non-limiting examples of such suitable dosages may be as low as 10⁹ vector genomes to as much as 10¹⁷ vector genomes (or “viral particles”) per administration.

In some aspects of the methods described herein, the number of viral particles (e.g., rAAV viral vectors) administered to the subject ranges from about 10⁹ to about 10¹⁷. In some aspects, about 10¹⁰ to about 10¹², about 10¹¹ to about 10¹³, about 10¹¹ to about 10¹², about 10¹¹ to about 10¹⁴, about 10¹² to about 10¹⁶, about 10¹³ to about 10¹⁶, about 10¹⁴ to about 10¹⁵, about 5×10¹¹ to about 5×10¹², or about 10¹² to about 10¹³, or about 10¹¹ to about 10¹⁸ viral particles are administered to the subject.

In some aspects of the methods described herein, the number of viral particles (e.g., rAAV viral vectors) administered to the subject is at least about 10¹⁰, or at least about 10¹¹, or at least about 10¹², or at least about 10¹³, or at least about 10¹⁴, or at least about 10¹⁵, or at least about 10¹⁶, or at least about 10¹⁷ viral particles.

In some aspects of the methods described herein, the number of viral particles (e.g., rAAV viral vectors) administered to the subject can depend on the age of the subject. In non-limiting examples, a subject that is 7 years of age or older can be administered about 10×10¹⁴ viral particles, a subject that is about 4 years of age to about 7 years of age can be administered about 10×10¹⁴ viral particles, a subject that is about 3 years of age to about 4 years of age can be administered about 9×10¹⁴ viral particles, a subject that is about 2 years of age to about 3 years of age can be about 8.2×10¹⁴ viral particles, a subject that is about 1 year of age to about 2 years of age can be administered about 7.3×10¹⁴ viral particles, a subject that is about 0.5 years of age to about 1 year of age can be administered about 4×10¹⁴ viral particles, or a subject that is less than 0.5 years of age can be administered 3×10¹⁴ viral particles.

In some aspects, the amounts of viral particles in a composition, pharmaceutical composition, or the amount of viral particles administered to a patient can calculated based on the percentage of viral particles that are predicted to contain viral genomes.

In some aspects, rAAV viral vectors of the present disclosure can be introduced to the subject intravenously, intrathecally, intracerebrally, intraventricularly, intranasally, intratracheally, intra-aurally, intra-ocularly, or peri-ocularly, orally, rectally, transmucosally, inhalationally, transdermally, parenterally, subcutaneously, intradermally, intramuscularly, intracisternally, intranervally, intrapleurally, topically, intralymphatically, intracisternally; such introduction may also be intra-arterial, intracardiac, subventricular, epidural, intracerebral, intracerebroventricular, sub-retinal, intravitreal, intraarticular, intraperitoneal, intrauterine, intranerve or any combination thereof. In some aspects, the viral particles are delivered to a desired target tissue, e.g., to the lung, eye, or CNS, as non-limiting examples. In some aspects, delivery of viral particles is systemic. The intracisternal route of administration involves administration of a drug directly into the cerebrospinal fluid of the brain ventricles. It could be performed by direct injection into the cisterna magna or via a permanently positioned tube. In some aspects, the rAAV viral vectors of the present disclosure are administered intrathecally.

Administration of the rAAV vectors, rAAV viral vectors, compositions or pharmaceutical compositions of this disclosure can be effected in one dose, continuously or intermittently throughout the course of treatment. In some aspects, the rAAV vectors, rAAV viral vectors, compositions, or pharmaceutical compositions of this disclosure are parenterally administered by injection, infusion, or implantation.

In some aspects, the rAAV viral vectors of this disclosure show enhanced tropism for brain and cervical spine. In some aspects, the rAAV viral vectors of the disclosure can cross the blood-brain-barrier (BBB).

### Methods of Manufacture

A variety of approaches may be used to produce or rAAV viral vectors of the present disclosure. In some aspects, packaging is achieved by using a helper virus or helper plasmid and a cell line. The helper virus or helper plasmid contains elements and sequences that facilitate viral vector production. In another aspect, the helper plasmid is stably incorporated into the genome of a packaging cell line, such that the packaging cell line does not require additional transfection with a helper plasmid.

In some aspects, the cell is a packaging or helper cell line. In some aspects, the helper cell line is eukaryotic cell; for example, an HEK 293 cell or 293T cell. In some aspects, the helper cell is a yeast cell or an insect cell.

In some aspects, the cell comprises a nucleic acid encoding a tetracycline activator protein; and a promoter that regulates expression of the tetracycline activator protein. In some aspects, the promoter that regulates expression of the tetracycline activator protein is a constitutive promoter. In some aspects, the promoter is a phosphoglycerate kinase promoter (PGK) or a CMV promoter.

A helper plasmid may comprise, for example, at least one viral helper DNA sequence derived from a replication-incompetent viral genome encoding in trans all virion proteins required to package a replication incompetent AAV, and for producing virion proteins capable of packaging the replication-incompetent AAV at high titer, without the production of replication-competent AAV.

Helper plasmids for packaging AAV are known in the art, see, e.g., U.S. Patent Pub. No. 2004/0235174 A1, incorporated herein by reference. As stated therein, an AAV helper plasmid may contain as helper virus DNA sequences, by way of non-limiting example, the Ad5 genes E2A, E4 and VA, controlled by their respective original promoters or by heterologous promoters. AAV helper plasmids may additionally contain an expression cassette for the expression of a marker protein such as a fluorescent protein to permit the simple detection of transfection of a desired target cell.

The disclosure provides methods of producing rAAV viral vectors comprising transfecting a packaging cell line with any one of the AAV helper plasmids disclosed herein; and any one of the rAAV vectors disclosed herein. In some aspects, the AAV helper plasmid and rAAV vector are co-transfected into the packaging cell line. In some aspects, the cell line is a mammalian cell line, for example, human embryonic kidney (HEK) 293 cell line. The disclosure provides cells comprising any one of the rAAV vectors and/or rAAV viral vectors disclosed herein.

As used herein, the term “helper” in reference to a virus or plasmid refers to a virus or plasmid used to provide the additional components necessary for replication and packaging of any one of the rAAV vectors disclosed herein. The components encoded by a helper virus may include any genes required for virion assembly, encapsidation, genome replication, and/or packaging. For example, the helper virus or plasmid may encode necessary enzymes for the replication of the viral genome. Non-limiting examples of helper viruses and plasmids suitable for use with AAV constructs include pHELP (plasmid), adenovirus (virus), or herpesvirus (virus). In some aspects, the pHELP plasmid may be the pHELPK plasmid, wherein the ampicillin expression cassette is exchanged with a kanamycin expression cassette.

As used herein, a packaging cell (or a helper cell) is a cell used to produce viral vectors. Producing recombinant AAV viral vectors (rAAV viral vectors) requires Rep and Cap proteins provided in trans as well as gene sequences from Adenovirus that help AAV replicate. In some aspects, Packaging/helper cells contain a plasmid is stably incorporated into the genome of the cell. In other aspects, the packaging cell may be transiently transfected. Typically, a packaging cell is a eukaryotic cell, such as a mammalian cell or an insect cell.

### Kits

The isolated polynucleotides, rAAV vectors, rAAV viral vectors, compositions, and/or pharmaceutical compositions described herein may be assembled into pharmaceutical or diagnostic or research kits to facilitate their use in therapeutic, diagnostic, or research applications. In some aspects, the kits of the present disclosure include any one of the isolated polynucleotides, rAAV vectors, rAAV viral vectors, compositions, pharmaceutical compositions, host cells, isolated tissues, as described herein.

In some aspects, a kit further comprises instructions for use. Specifically, such kits may include one or more agents described herein, along with instructions describing the intended application and the proper use of these agents. In some aspects, the kit may include instructions for mixing one or more components of the kit and/or isolating and mixing a sample and applying to a subject. In some aspects, agents in a kit are in a pharmaceutical formulation and dosage suitable for a particular application and for a method of administration of the agents. Kits for research purposes may contain the components in appropriate concentrations or quantities for running various experiments.

The kit may be designed to facilitate use of the methods described herein and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. In some aspects, the compositions may be provided in a preservation solution (e.g., cryopreservation solution). Non-limiting examples of preservation solutions include DMSO, paraformaldehyde, and CryoStor® (Stem Cell Technologies, Vancouver, Canada). In some aspects, the preservation solution contains an amount of metalloprotease inhibitors.

In some aspects, the kit contains any one or more of the components described herein in one or more containers. Thus, in some aspects, the kit may include a container housing agents described herein. The agents may be in the form of a liquid, gel or solid (powder). The agents may be prepared sterilely, packaged in a syringe and shipped refrigerated. Alternatively, they may be housed in a vial or other container for storage. A second container may have other agents prepared sterilely. Alternatively, the kit may include the active agents premixed and shipped in a syringe, vial, tube, or other container. The kit may have one or more or all of the components required to administer the agents to a subject, such as a syringe, topical application devices, or IV needle tubing and bag.

### Further Definitions

Unless the context indicates otherwise, it is specifically intended that the various features of the invention described herein can be used in any combination. Moreover, the disclosure also contemplates that, in some aspects, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified aspects, embodiments, features, and terms intend to include both the recited aspect, embodiment, feature, or term and biological equivalents thereof.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2nd edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (RI. Freshney, ed. (1987)).

As used herein, the term “comprising” is intended to mean that the compositions and methods include the recited elements, but do not exclude others. As used herein, the transitional phrase “consisting essentially of” (and grammatical variants) is to be interpreted as encompassing the recited materials or steps and those that do not materially affect the basic and novel characteristic(s) of the recited embodiment. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the present disclosure. In each instance herein any of the terms “comprising,” “consisting essentially of,” and “consisting of” can be replaced with either of the other two terms, while retaining their ordinary meanings.

All numerical designations, e.g., pH, temperature, time, concentration, and molecular weight, including ranges, are approximations which are varied (+) or (−) by increments of 1.0 or 0.1, as appropriate, or, alternatively, by a variation of +/−15%, 10%, 5%, 2%. It is to be understood, although not always explicitly stated, that all numerical designations are preceded by the term “about”. It also is to be understood, although not always explicitly stated, that the reagents described herein are merely exemplary and that equivalents of such are known in the art. The term “about,” as used herein when referring to a measurable value such as an amount or concentration and the like, is meant to encompass variations of 20%, 10%, 5%, 1%, 0.5%, or even 0.1 of the specified amount. In some embodiments, “about” encompasses variations of up to 10 of the specified amount.

The terms “acceptable,” “effective,” or “sufficient” when used to describe the selection of any components, ranges, dose forms, etc. disclosed herein intend that said component, range, dose form, etc. is suitable for the disclosed purpose.

Also, as used herein, “and/or” refers to and encompasses any and all possible combinations of one or more of the associated listed items, as well as the lack of combinations when interpreted in the alternative (“or”).

Unless specifically recited, the term “host cell” includes a eukaryotic host cell, including, for example, fungal cells, yeast cells, higher plant cells, insect cells and mammalian cells. Non-limiting examples of eukaryotic host cells include simian, bovine, porcine, murine, rat, avian, reptilian and human, e.g., HEK293 cells and 293T cells.

The term “isolated” as used herein refers to molecules or biologicals or cellular materials being substantially free from other materials.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising, consisting essentially of, or consisting of purine and pyrimidine bases or other natural, chemically or biochemically modified, non-natural, or derivatized nucleotide bases.

A “gene” refers to a polynucleotide containing at least one open reading frame (ORF) that is capable of encoding a particular polypeptide or protein. A “gene product” or, alternatively, a “gene expression product” refers to the amino acid sequence (e.g., peptide or polypeptide) generated when a gene is transcribed and translated.

As used herein, “expression” refers to the two-step process by which polynucleotides are transcribed into mRNA and/or the process by which the transcribed mRNA is subsequently translated into peptides, polypeptides, or proteins. If the polynucleotide is derived from genomic DNA, expression may include splicing of the mRNA in a eukaryotic cell.

“Under transcriptional control” is a term well understood in the art and indicates that transcription of a polynucleotide sequence, usually a DNA sequence, depends on its being operatively linked to an element that contributes to the initiation of, or promotes, transcription. “Operatively linked” intends that the polynucleotides are arranged in a manner that allows them to function in a cell. In one aspect, promoters can be operatively linked to the downstream sequences.

The term “encode” as it is applied to polynucleotide sequences and/or nucleic acid sequences refers to a polynucleotide sequences and/or nucleic acid sequences which are said to “encode” an RNA molecule (e.g. an shRNA molecule) if, in their native state, the of the polynucleotide sequence and/or nucleic acid sequence corresponds the sequence of the shRNA molecule that is biologically active. The antisense strand is the complement of such a polynucleotide sequence and/or nucleic acid sequence, and the encoding sequence can be deduced therefrom.

The terms “equivalent” or “biological equivalent” are used interchangeably when referring to a particular molecule, biological material, or cellular material and intend those having minimal homology while still maintaining desired structure or functionality. Non-limiting examples of equivalent polypeptides include a polypeptide having at least about 60%, at least about 65%, at least about 70%, at least about 75%, at least about 80%, at least about 85%, at least about 90%, at least about 95% identity or at least about 99% identity to a reference polypeptide (for instance, a wild-type polypeptide); or a polypeptide which is encoded by a polynucleotide having at least about 70%, at least about 75%, at least about 80%, at least about 85%, at least about 90%, at least about 95% identity, at least about 97% sequence identity or at least about 99% sequence identity to the reference polynucleotide (for instance, a wild-type polynucleotide).

“Homology” or “identity” or “similarity” refers to sequence similarity between two peptides or between two nucleic acid molecules. Percent identity can be determined by comparing a position in each sequence that may be aligned for purposes of comparison. When a position in the compared sequence is occupied by the same base or amino acid, then the molecules are identical at that position. A degree of identity between sequences is a function of the number of matching positions shared by the sequences. “Unrelated” or “non-homologous” sequences share less than 40% identity, less than 25% identity, with one of the sequences of the present disclosure. Alignment and percent sequence identity may be determined for the nucleic acid or amino acid sequences provided herein by importing said nucleic acid or amino acid sequences into and using ClustalW (available at https://genome.jp/tools-bin/clustalw/). For example, the ClustalW parameters used for performing the protein sequence alignments found herein were generated using the Gonnet (for protein) weight matrix. In some aspects, the ClustalW parameters used for performing nucleic acid sequence alignments using the nucleic acid sequences found herein are generated using the ClustalW (for DNA) weight matrix.

A polynucleotide disclosed herein can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “plasmid” is a DNA molecule that is typically separate from and capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or, alternatively, the proteins produced may act as toxins under similar circumstances. It is known in the art that while plasmid vectors often exist as extrachromosomal circular DNA molecules, plasmid vectors may also be designed to be stably integrated into a host chromosome either randomly or in a targeted manner, and such integration may be accomplished using either a circular plasmid or a plasmid that has been linearized prior to introduction into the host cell.

“Plasmids” used in genetic engineering are called “plasmid vectors”. Many plasmids are commercially available for such uses. The gene to be replicated is inserted into copies of a plasmid containing genes that make cells resistant to particular antibiotics, and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria or eukaryotic cells containing a plasmid harboring the gene of interest, which can be induced to produce large amounts of proteins from the inserted gene.

In aspects where gene transfer is mediated by a DNA viral vector, such as an adenovirus (Ad) or adeno-associated virus (AAV), a vector construct refers to the polynucleotide comprising, consisting essentially of, or consisting of the viral genome or part thereof, and a transgene/exogenous polynucleotide sequence.

The term “tissue” is used herein to refer to tissue of a living or deceased organism or any tissue derived from or designed to mimic a living or deceased organism. The tissue may be healthy, diseased, and/or have genetic mutations. The biological tissue may include any single tissue (e.g., a collection of cells that may be interconnected), or a group of tissues making up an organ or part or region of the body of an organism. The tissue may comprise, consist essentially of, or consist of a homogeneous cellular material or it may be a composite structure such as that found in regions of the body including the thorax which for instance can include lung tissue, skeletal tissue, and/or muscle tissue. Exemplary tissues include, but are not limited to those derived from liver, lung, thyroid, skin, pancreas, blood vessels, bladder, kidneys, brain, biliary tree, duodenum, abdominal aorta, iliac vein, heart and intestines, including any combination thereof.

All references cited herein are incorporated herein by reference in their entirety.

### EXAMPLES

The examples described in this section is provided for illustration only and is in no way intended to limit the invention.

### Example 1: AAV Mediated miRNA Knockdown of Gys1 RNA Reduces Pathogenic Polyglucosan Bodies and Neuroinflammation in Adult Polyglucosan Body and Lafora Disease Mouse Models

This example describes an adeno-associated virus vector (AAV) mediated RNA-interference (RNAi) strategy to reduce glycogen synthase mRNA and PBs across the brain in APBD and LD mouse models. amiRNAs have several advantages over earlier-generation short hairpin loop RNAs. Without wishing to be bound by theory, earlier-generation short hairpin loop RNAs are believed to crowd out the complex that dices out their loops (DICER), leading to deficient processing of endogenous miRNAs and resultant toxicity. amiRNAs, on the other hand, are believed to be pre-processed in a regulated fashion to occupy DICER in regular order without disturbing the processing of other miRNAs (Boudreau et al., Mol Ther. 2009; 17(1):169-75; Toro Cabrera et al., Methods Mol Biol. 2016; 1382:67-80).

### Methods

### Study Design

The Gys1-targeting AAV-amiRNA, or phosphate-buffered saline (PBS), was administered at postnatal day 2 by intracerebroventricular (ICV) injection. Mice were sacrificed at 3 months of age, and the effects of the treatment on PB quantity and neuroinflammatory markers were analyzed.

### Plasmid Construction and Viral Packaging

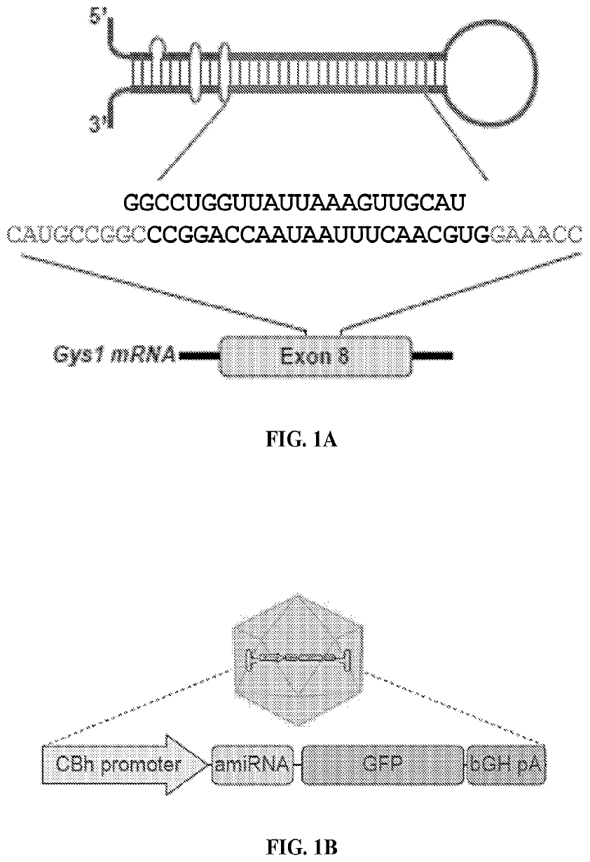

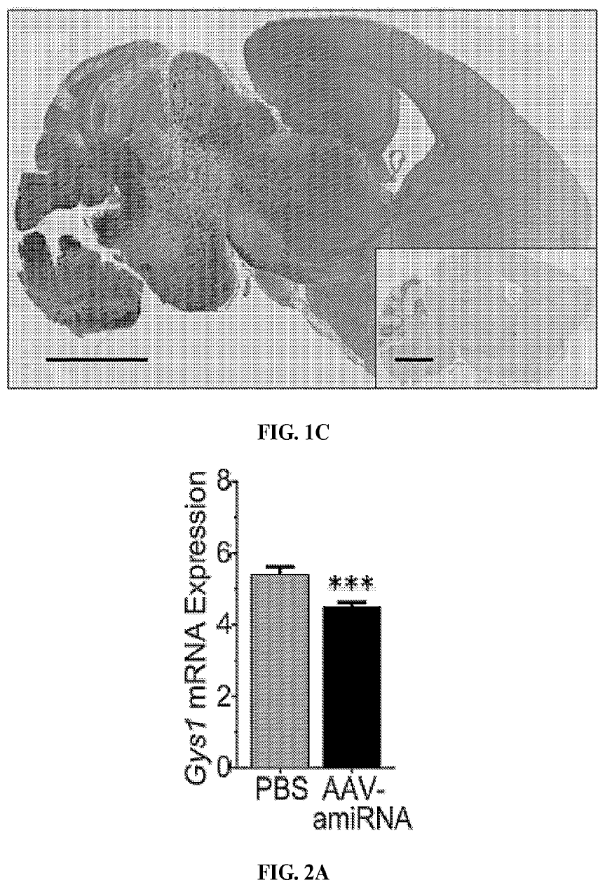

Gys1 targeting siRNA sequences were predicted using siSPOTR (Boudreau et al., Methods Enzymol. 2012; 507:275-96). Using Gibson Assembly (NEB), these were cloned into the natural human/mouse miR-30 pri-miRNA scaffold (Boudreau et al., Methods Enzymol. 2012; 507:275-96) and a green fluorescent protein (GFP) coding sequence containing AAV based expression vector (Gray et al., Hum Gene Ther. 2011; 22(9):1143-53; FIGS. 1A and 1B). The CBh promoter was used to drive expression of the amiRNA and GFP simultaneously. A 3′ bGH poly(A) signal stabilized the transcript (FIG. 1B). The cassette was flanked by AAV2 inverted terminal repeats and cloned in self-complementary fashion for enhanced post-transduction stability and expression. The final plasmid was packaged in AAV9 as described in Clement et al., Mol Ther Methods Clin Dev. 2016; 3:16002.

### Mice

APBD (Gbe1^{Y239S}) and LD (Epm2a^{-/-} and Epm2b^{-/-}) mouse models were described previously (see Ganesh et al., Human Molecular Genetics, 2002, 11(11): 1251-1262; Turnbull et al., Ann Neurol. 2010 December; 68(6):925-33; Akman et al., Human Molecular Genetics, 2015, 24(23): 6801-6810). Both sexes were used in approximately equal proportion in all experiments. 5×10¹¹ vector genomes (in 5 μl solution), or 5 μl PBS, were injected ICV, as described in Kawasaki et al., J Vis Exp. 2016(113). Mice were sacrificed by cervical dislocation, and brains were harvested and cut into two hemispheres, one fixed in formalin for paraffin embedding and histo- and immunohistochemistry, the other snap-frozen in liquid nitrogen, later ground into powder by mortar and pestle, and aliquoted into 30-40 mg powder in screwcap tubes for the genetic and biochemical experiments below.

### GFP Immunohistochemistry

Sections (5 μm) were mounted on glass slides, de-paraffinized and rehydrated by processing through xylene and decreasing concentrations of ethanol in water, and subjected to antigen retrieval using citrate buffer pH 6.0 (Sigma). Endogenous peroxidase activity was blocked for 10 min with BLOXALL solution (Vector labs). Sections were incubated with rabbit anti-GFP antibody (1:50, Santa Cruz) diluted in normal horse serum overnight at 4° C., then successively incubated with Amplifier Antibody and ImmPRESS Polymer Reagent (Vector labs) and the ImmPACT DAB EqV working solution (Vector labs) until desired stain intensity.

Gys1 mRNA Extraction and Quantification by Droplet Digital PCR

RNA was extracted using TriZol (Invitrogen) and purified using PureLink RNA Mini Kit (Invitrogen) following manufacturer instructions. cDNA was generated using the iScript Reverse Transcription SuperMix kit (Bio-Rad Laboratories). Gys1 RNA was quantified using the Bio-Rad QX200 Droplet Digital PCR (ddPCR) system as instructed by the manufacturer. Tfrc was used as reference gene. Custom designed TaqMan primers and probes (Thermo Fisher Scientific) were used for both Gys1 and Tfrc. ddPCR reactions were assembled using standard protocols. The 20 μL reaction mix consisted of 10 μL 2×ddPCR SuperMix for Probes (Bio-Rad Laboratories), 1 μL Gys1 assay mix (FAM-labeled), 1 μL reference Tfrc assay mix (VIC-labeled), 3 μL of cDNA, and 5 μL nuclease-free water. Cycling conditions were 95° C. for 10 min, 45 cycles of 94° C. for 30 see and 60° C. for 1 min, and 98° C. for 10 min. The resulting data was quantified by QuantaSoft v 1.4 (Bio-Rad). Results were expressed as ratio of Gys1/Tfrc. No-template controls were run in parallel with study samples. Primer/probe sets are listed in Table 1.

### GFP and GYS1 Western Blots

Tissue lysate was obtained by homogenizing frozen ground brain tissue using ice-cold RIPA lysis buffer (NaCl 150 mM, NP-40 1%, Sodium deoxycholate 0.5%, SDS 0.1%, Tris HCl 50 mM pH 8.0) containing protease inhibitors (1 mM PMSF, 5 μg/mL Leupetin, 10 μg/mLPepstatin, 20 KIU/mL Aprotinin, 50 mM NaF). Lysates were centrifuged at 14,000×g for 5 min at 4° C. and supernatants collected. Protein concentration was measured using the Bradford assay reagent (ThermoScientific). Standard curve for protein quantification was generated using serial dilution of albumin standard (ThermoScientific). Equal amounts of whole protein from each sample was subjected to SDS-PAGE using the TGX Stain-Free FastCast Acrylamide kit (BioRad). Protein bands were transferred to polyvinylidene difluoride (PVDF) membrane (Millipore) overnight at 4° C. Primary antibodies for GFP (1:1000, Santa Cruz) and for GYS1 (1:1000, Cell Signalling) were used. Protein densitometry was performed using Image Lab software (Bio-Rad Laboratories). Intensity of each protein band was normalized to the intensity of its corresponding whole protein lane image obtained on the same membrane.

### PASD Staining and LB Quantitation

Paraffin-embedded brain tissue was sectioned and stained using the periodic acid-Schiff diastase (PASD) method as described previously [19, 21]. Stained slides were scanned using the Hamamatsu Nanozoomer 2.0 HT digital slide scanner (40×objective), and the % area of hippocampus covered by LBs was quantified, as reported previously [24], using HistoQuant (3DHistech) by defining LBs signals based on pixel color. Values are expressed as % area.

### Quantification of Degradation-Resistant Glycogen

Aliquots of frozen ground brain were left out to thaw at room temperature for 1 hour to allow soluble glycogen to degrade, leaving behind degradation-resistant glycogen, as previously described [30]. The sample was then boiled in 30% KOH and glycogen precipitated in 70% ethanol with 57 mM sodium sulfate. Three further rounds of centrifugation and suspension in 67% ethanol with 15 mM LiCl followed, after which the pellet was resuspended in 100 mM sodium acetate pH 4.5 and digested with amyloglucosidase (Megazyme) in a 55° C. oven for 1 hour along with digestion of blank controls. Glucose amount was determined in triplicate for each sample using a modified version of Lowry and Passonneau. Briefly, samples and glucose standards were mixed with 170 μL of G6PDH reaction mix (200 mM tricine buffer/10 mM MgCl₂ pH 8, 0.66 mM NADP; 1 mM of ATP; 0.5 units of G6PDH). Absorbance at 340 nm was acquired before (Abs1) and after (Abs2) addition of hexokinase (0.6 units in 4 μl of 200 mM Tricine/KOH, pH 8, 10 mM MgCl₂). (Abs2−Abs1) of glucose standards was used to derive glucose quantity in samples. Following normalization to fresh weight, glucose quantity represents degradation-resistant glycogen.

### Quantitative Real-Time PCR

RNA was extracted as described above. Immune system genes Cxcl10, Ccl5, Lcn2 and C3 [5, 10] were quantified by quantitative real-time PCR (qRT-PCR) using the QuantStudio 7 Pro System thermo-cycler (ThermoFischer Scientific) and SYBR Green Master Mix (Bio-Rad Laboratories). Data are shown as fold change relative to control samples using the ΔΔC_{q} method with Rpl4 as an internal control gene. Primers are listed in Table 4.

TABLE 4
Primer and probe sequences used in this study.

| Gene | Forward Primer (5′ → 3′) | Reverse Primer (5′ → 3′) |
|---|---|---|
| Gys1 (ddPCR) | CAGAGCAAAGCACGAATCCA | CATAGCGGCCAGCGATAAAG |
| | (SEQ ID NO: 42) | (SEQ ID NO: 43) |
| Gysl Probe | TTATGGGCACCTGGAC | |
| (ddPCR) | (SEQ ID NO: 44) | |
| RTfrc (ddPCR) | TGCCTAATATACCTGTGCAA | TTCCTTCCATTTTTCCAAATAG |
| | ACAATC (SEQ ID NO: 45) | CT (SEQ ID NO: 46) |
| Tfrc Probe | CAAGAGCTGCTGCAGAA | |
| (ddPCR) | (SEQ ID NO: 47) | |
| Rpl4 | CCCTTACGCCAAGACTATGC | TGGAACAACCTTCTCGGATT |
| | (SEQ ID NO: 48) | (SEQ ID NO: 49) |
| Cxcl10 | AAGTGCTGCCGTCATTTTCT | ATAGGCTCGCAGGGATGATT |
| | (SEQ ID NO: 50) | (SEQ ID NO: 51) |
| Ccl5 | TGCCAACCCAGAGAAGAAGT | AGCAAGCAATGACAGGGAAG |
| | (SEQ ID NO: 52) | (SEQ ID NO: 53) |
| Lcn2 | GCCTCAAGGACGACAACATC | CACACTCACCACCCATTCAG |
| | (SEQ ID NO: 54) | (SEQ ID NO: 55) |
| C3 | CTGTGTGGGTGGATGTGAAG | TCCTGAGTGTCGTTTGTTGC |
| | (SEQ ID NO: 56) | (SEQ ID NO: 57) |

### Statistical Analysis

Data were analyzed and graphed using the GraphPad Prism software (v. 8.0.2; GraphPad Software). One-way ANOVA with Tukey's post-hoc test was performed for multiple comparisons. Student's unpaired t-test was used to compare single means. For all comparisons, statistical significance was set at p<0.05. Asterisks denote level of significance based on p value: *p<0.05, **p<0.01, ***p<0.001, and ****p<0.0001.

### Results

Design and Validation of the Artificial miRNA Construct

The amiRNA construct contains a Gys1-targeting double-stranded exogenous small RNA sequence embedded in an endogenous miRNA backbone (FIG. 1A). The sequence of the GYS-1 targeting sequences tested were UACGUUGAAAUUAUUGGUCCGG (SEQ ID NO: 40) and GAUUCGUAGAGCUUCCUCCCAA (SEQ ID NO: 41).

The functionality of multiple amiRNA constructs was tested in cell lines. N2A cells were transfected and Gys1 mRNA levels were quantified using qRT-PCR and transduction efficiencies were quantified using GFP fluorescence. The most potent amiRNA was selected for animal studies.

To confirm murine brain virus transduction and to assess transduction pattern and distribution, immunohistochemistry with an anti-GFP antibody was performed. GFP expression was strong and broadly distributed across the brain in all three mouse models (FIG. 1C).

AAV-amiRNA Decreases Gys1 mRNA and Protein Levels

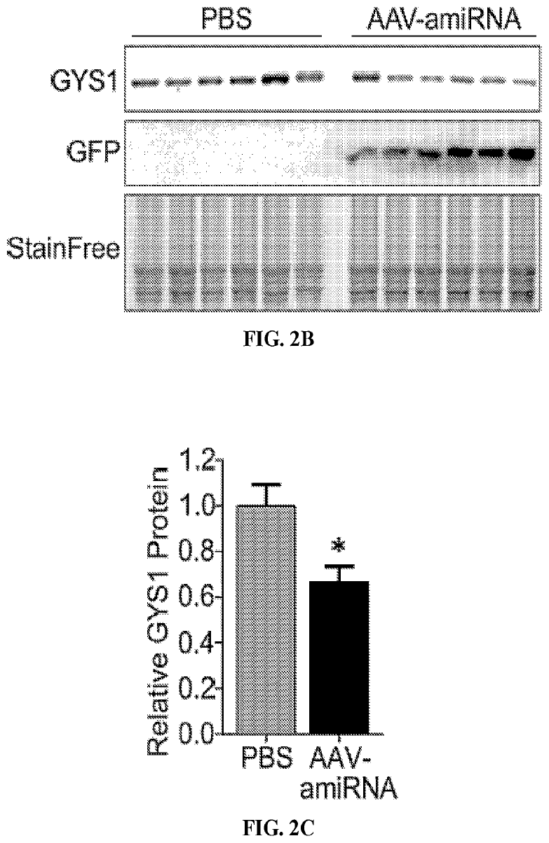

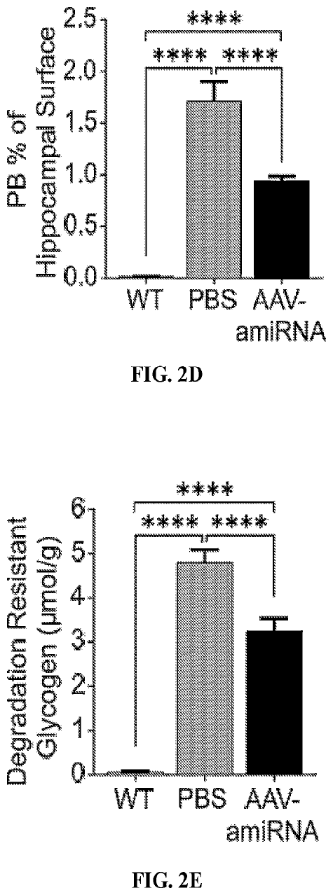

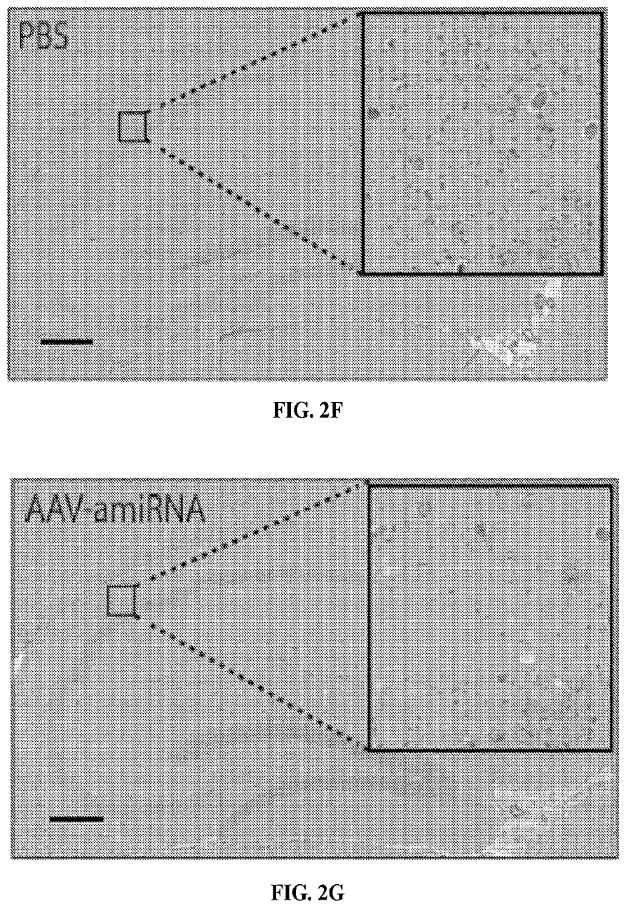

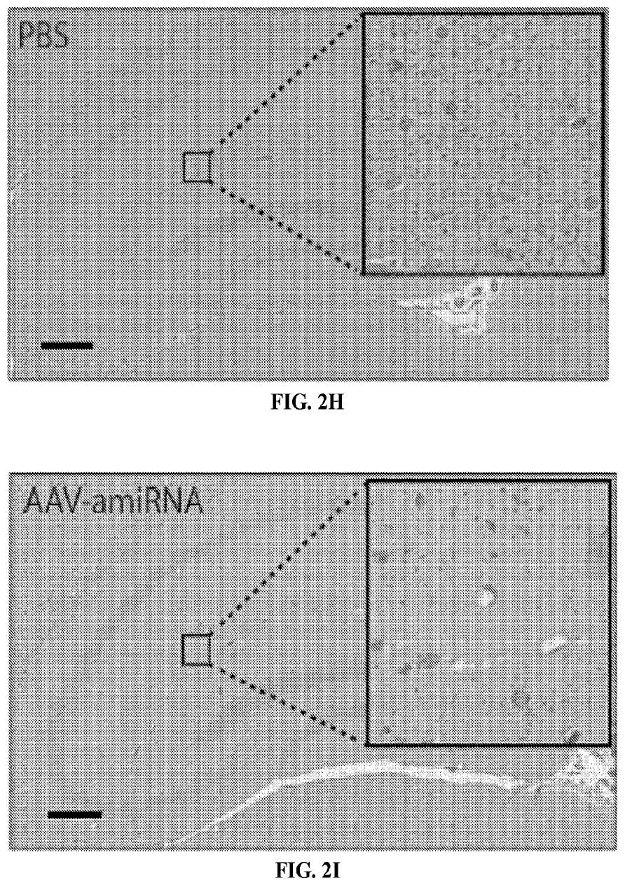

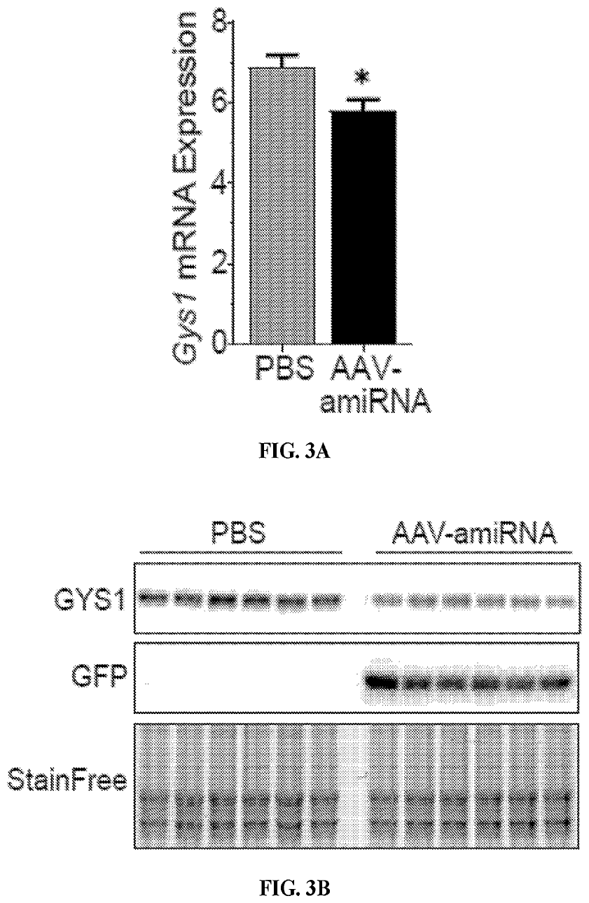

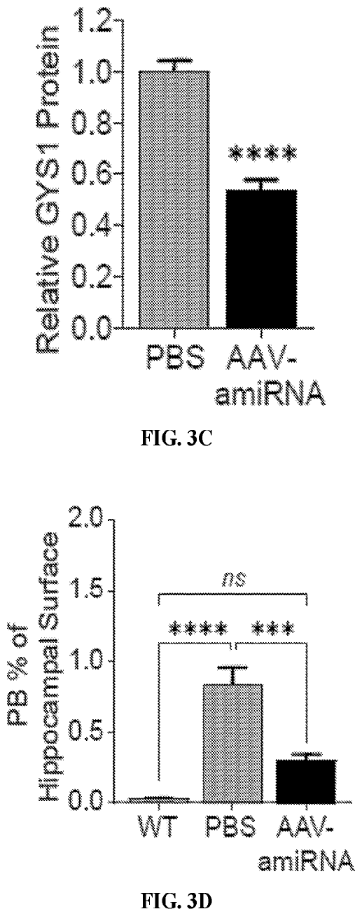

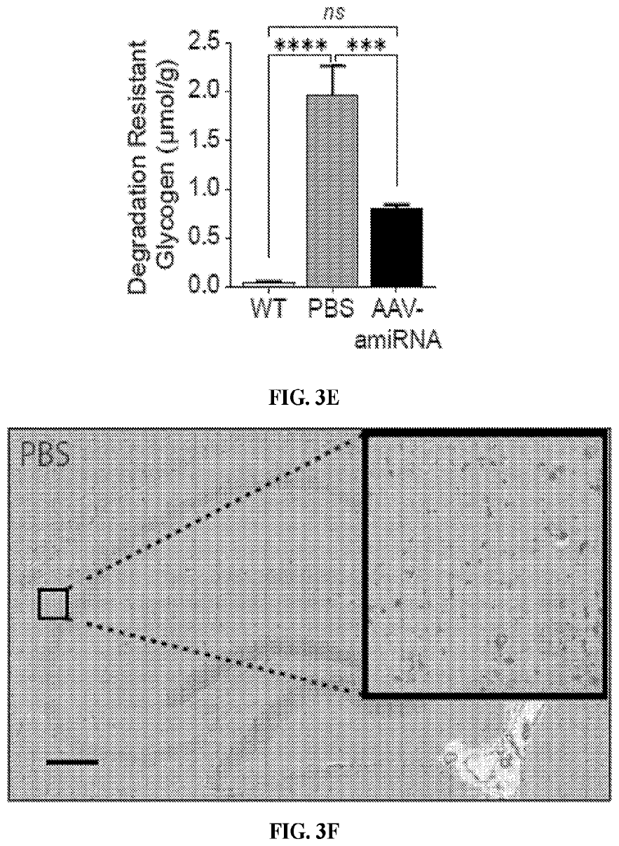

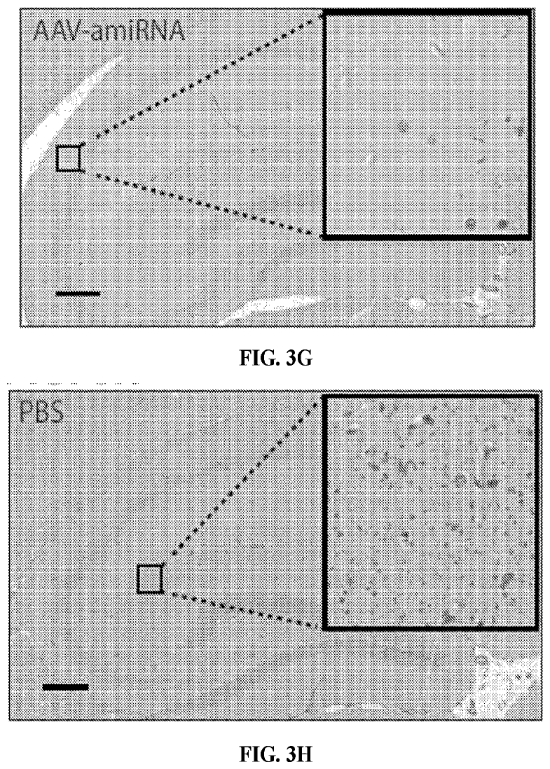

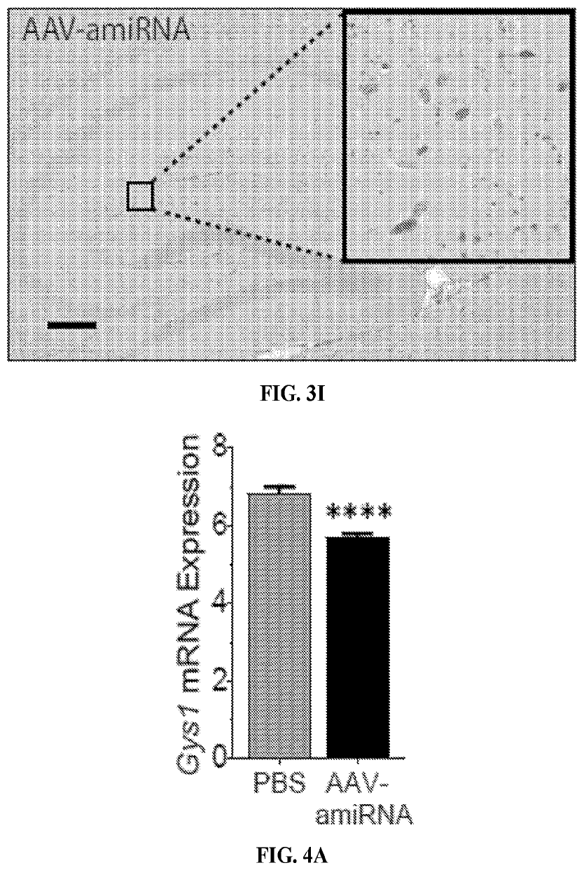

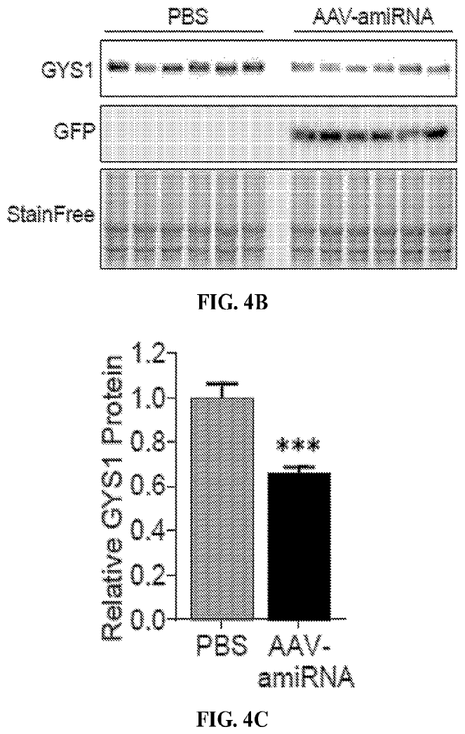

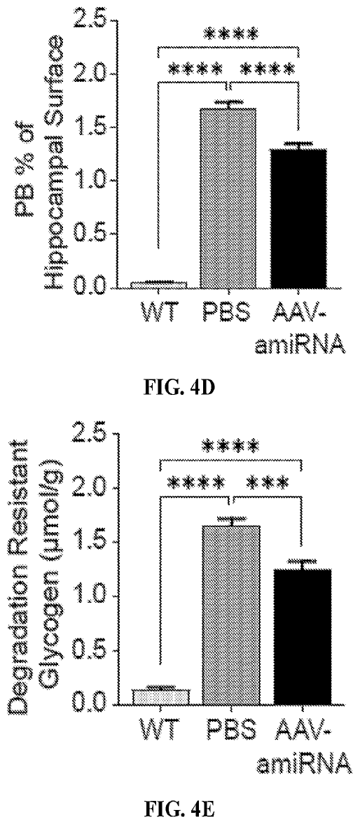

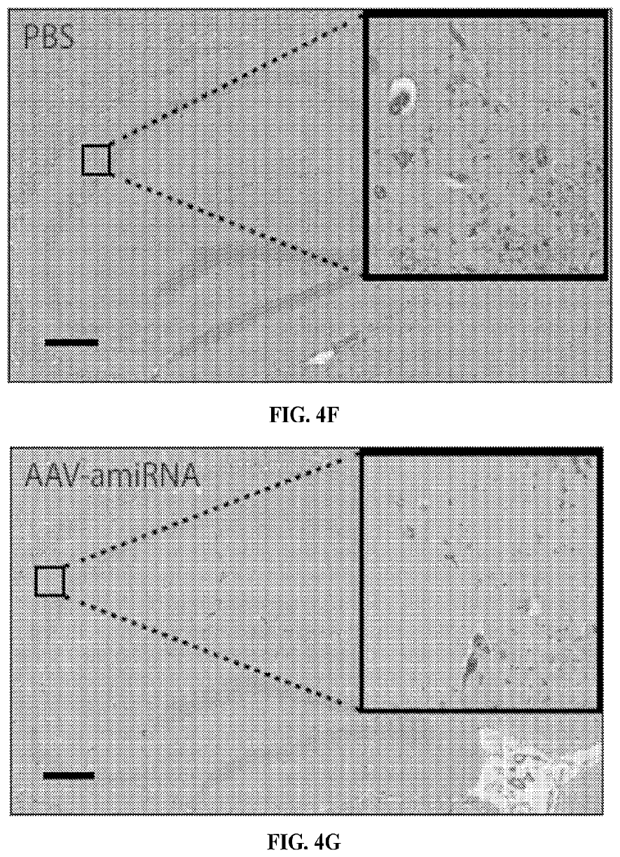

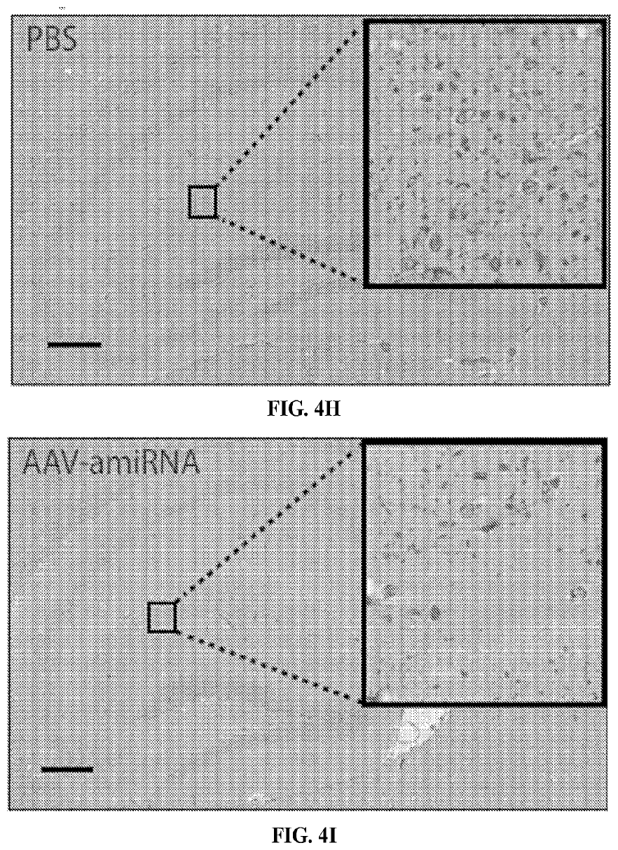

To quantify the degree of Gys1 mRNA knockdown in brain, ddPCR was performed on whole hemisphere extracts. The AAV-amiRNA-treated mice of all three genotypes showed an approximately 13 to 18% reduction in Gys1 mRNA (FIGS. 2A, 3A, and 4A). Quantitative western blotting on the same extracts showed a larger, 40 to 50%, reduction of GYS1 protein (FIGS. 2B, 2C, 3B, 3C, 4B, and 4C).

### AAV-amiRNA Reduces PBs and Degradation-Resistant Glycogen

At three months of age, PBs are clearly visible throughout the brain in all three disease models, and changes in PB amounts can be quantified histologically (Gumusgoz et al., Models. Neurotherapeutics. 2021; 18(2):1414-25; Ahonen et al., Brain. 2021). To measure the effect of AAV-amiRNA on PBs, sections from paraffin embedded hemispheres were stained with PASD and the % PASD signal per hippocampal area was quantified. AAV-amiRNA treated mice showed 40% to 60% reduced PB accumulations in the hippocampus compared to vehicle treated mice (FIGS. 2D, 2F-2I, 3D, 3F-3I, 4D, and 4F-4I).

Brain glycogen in APBD and LD comprises normal water-soluble glycogen and precipitated and aggregated glycogen (polyglucosans, PBs) (Souza et al., J Inherit Metab Dis. 2021; 44(3):534-43; Mitra et al., Rev Neurol (Paris) 2021; Sullivan et al., Int J Mol Sci. 2017; 18(8)). The soluble portion degrades at room temperature, while the insoluble does not. Biochemical quantification of the latter is a second standard way to measure PB load (Israelian et al., J Neurochem. 2021; 157(6):1897-910). AAV-amiRNA treated mice had 40 to 60% reduced degradation-resistant glycogen in whole-hemisphere extracts compare to vehicle treated mice (FIGS. 2E, 3E, and 4E), comparable to the PB reductions as measured histochemically.

### AAV-amiRNA Ameliorates PB-Associated Immune Activation

The LD mouse models are full knockouts of the Epm2a or Epm2b genes. They recapitulate the neuropathology of LD. The PBs appear first and are well-established by 3 months of age. At 16 months, there is astrogliosis and microgliosis, and transcriptomic studies show that 94 of misregulated mRNAs are immune or inflammatory pathway gene transcripts (Lahuerta et al., Mol Neurobiol. 2020; 57(3):1607-21). At this age, there are behavioral deficits, myoclonus, seizures and seizure susceptibility to proconvulsant drugs (Ganesh et al., Hum Mol Genet. 2002; 11(11):1251-62; Turnbull et al., Ann Neurol. 2010; 68(6):925-33).

The APBD mouse model has the most common human APBD mutation, Tyr239Ser (Orhan Akman et al., Hum Mol Genet. 2015; 24(23):6801-10). It exhibits larger amounts of PBs than LD by 3 months of age, and by 8 months astrogliosis, microgliosis and behavioral and motor deficits. Transcriptomics have not been carried out, but three (Cxcl10, Lcn2, and C3) of the above four neuroimmune genes are upregulated at 3 months of age.

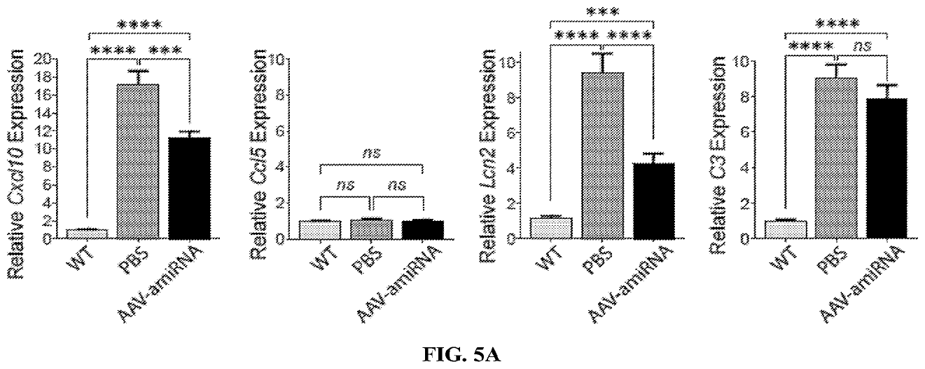

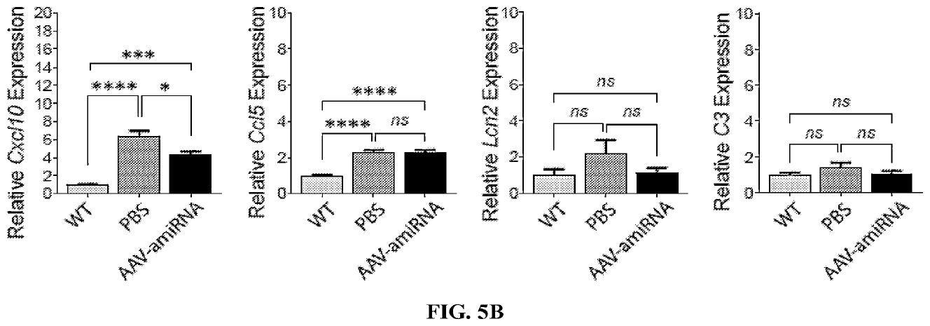

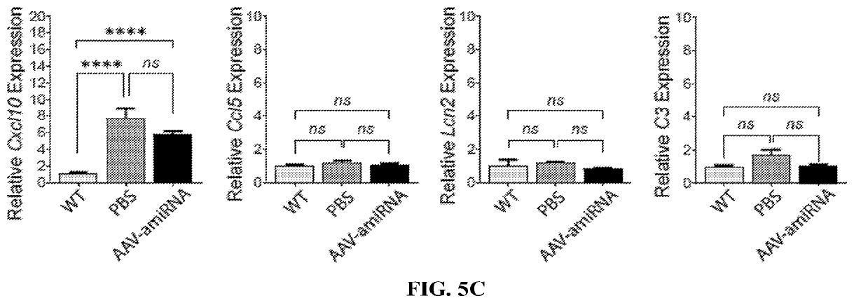

The expressions of Cxcl10, Ccl5, Lcn2 and C3 in wild-type and AAV-amiRNA or vehicle treated APBD and LD mice was quantified and upregulation of Cxcl10, Lcn2, and C3 in the APBD mice was confirmed. Expressions of two of these genes, Cxcl10 and Lcn2, was significantly reduced with the Gys1-targeting AAV-amiRNA. Levels of C3 also decreased, although not to statistical significance (FIG. 5A). In the Epm2a^{-/-} mice, two genes, Cxcl10 and Ccl5, were upregulated, and the level of Cxcl10 significantly corrected with the treatment (FIG. 5B). In the Epm2b^{-/-} mice, one gene, Cxcl10, was upregulated. This reduced with the treatment but not to statistical significance (FIG. 5C).

### Discussion

Downregulating glycogen synthase has emerged as a potential path to treating APBD and LD. Transgenic and CRISPR/Cas9 based knockout and knockdown experiments [5, 16-23] laid the groundwork for the development of an antisense oligonucleotide (ASO)-based therapy (Ahonen et al., Brain. 2021) and small molecule inhibitors of the glycogen synthase enzyme as potential oral drugs (Tang et al., J Med Chem. 2020; 63(7):3538-51). The main drawback for an ASO therapy is that it needs to be administered through repeat lumbar punctures multiples times a year lifelong. Challenges that face small molecules include that they do not immediately have the specificities of gene based therapies and, as in this case, require further development to allow blood brain barrier passage. A gene therapy based GYS1-directed single lifetime dose treatment for APBD and LD would overcome these limitations.

The core sequence of the amiRNA described in this example targets murine Gys1 RNA. Illustrative oligonucleotides that downregulate human GYS1 are shown in Table 1.

An important observation from the study described in this example is the disparity between the level of Gys1 mRNA reduction (13-18%) and the reductions of Gys1 protein, abnormal glycogen and PBs (40-60%) (FIGS. 2A-5I). This same disparity was noted in two other recent studies where Gys1 was downregulated by other means (Gumusgoz 2021; Nitschke 2021). Disconnect between expression and translation is not common but certainly not unknown (Maier et al., FEBS Lett. 2009; 583(24):3966-73). A practical implication of this disconnect is that the level of GYS1 protein appears to be exquisitely responsive to changes in the level of its mRNA, which could provide important advantage in clinical applications; for example, lower doses of viral genome of AAV-amiRNA may be required to achieve the desired result.

The basis of downregulating glycogen synthase for PB diseases is to rebalance the activities of glycogen synthase and branching enzyme and thus prevent the former from outpacing the latter and forming overlong glycogen branches. However, glycogen synthase downregulating therapies could also benefit others of the more than 15 glycogen storage diseases where normally structured (branched) glycogen accumulates in abnormally large amounts. By far the most common of these is Pompe disease due to acid maltase deficiency and accumulation of normally branched glycogen in lysosomes.

In summary, a virally delivered glycogen synthase downregulating miRNA effectively reduced the pathogenic PBs in the prototypical PB diseases APBD and LD, along with corrections of their early neuroimmune abnormalities.

### Example 2: Knockdown of GYS1 in HEK93 Cells Using amiRNA Molecule Directed Against Human GYS1

HEK293 human cells were transfected with amiRNA molecule directed against human GYS1. Cells were harvested 48 h after transfection, and GYS1 mRNA levels were determined using qPCR.

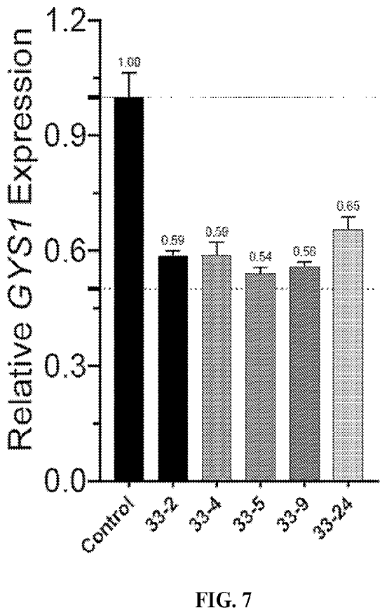

The transfection efficiency was ˜50-60%. The knockdown of GYS1 is shown in FIG. 7. The dotted line in FIG. 7 represents the 50% knockdown level. “33-2,” “33-4,” “33-5,” “33-9,” and “33-24” indicate the amiRNA2-33, amiRNA4-33, amiRNA5-33, amiRNA9-33, and amiRNA24-33 AAV vectors shown in Table 3, respectively.

### Methods

### Cell Culture and Transfection:

HEK293 cells (ATCC) were grown in DMEM (Gibco) containing 10% FBS and maintained at 37° C., with 5% CO2 under humidified condition. Cells were transfected with above amiRNA plasmids using Lipofectamine 3000 (ThermoFischer Scientific) following manufacturer instructions.

Total mRNA Extraction and Gys1 mRNA Quantification Using qPCR

Cells were harvested 48 hours after transfection. Total RNA was extracted using TriZol (Invitrogen) and purified using PureLink RNA Mini Kit (Invitrogen) following manufacturer instructions. cDNA was synthesized by iScript Reverse Transcription SuperMix kit (Bio-Rad Laboratories). Human Gys1 RNA was quantified using quantitative real-time PCR (qRT-PCR) using the QuantStudio 7 Pro System thermo-cycler (ThermoFischer Scientific) and SYBR Green Master Mix (Bio-Rad Laboratories). Following primers were used in qPCR: Gys1 forward 5′-CCGCTATGAGTTCTCCAACAAGG-3 (SEQ ID NO: 58) and reverse 5′-AGAAGGCAACCACTGTCTGCTC-3′ (SEQ ID NO: 59); GAPDH forward 5′-GCCACATCGCTCAGACAC-3 (SEQ ID NO: 60)′ and reverse 5′-GGCAACAATATCCACTTTACCAG-3′ (SEQ ID NO: 61). Data are shown as fold change relative to control (un-transfected HEK293 cells) using the ΔΔC_{q} method with GAPDH as an internal control gene.

What is claimed is:

1. An rAAV vector comprising at least one polynucleotide sequence encoding at least one artificial micro RNA (amiRNA) directed against GYS1, wherein the at least one amiRNA directed against GYS1 comprises the nucleic acid sequence set forth in any one of SEQ ID NOs: 35-39.

2. The rAAV vector of claim 1, wherein the at least one polynucleotide comprises the nucleic acid sequences set forth in any one of SEQ ID NOS: 1-5.

3. An rAAV vector of claim 1 or 2, wherein the rAAV vector further comprises a first AAV ITR sequence.

4. The rAAV vector of claim 3, wherein the first AAV ITR sequence is an AAV2 ITR sequence.

5. The rAAV vector of claim 3 or 4, wherein the first AAV ITR sequence comprises the sequence set forth in SEQ ID NO: 14

6. An rAAV vector of any one of claim 1-5, wherein the rAAV vector further comprises a second AAV ITR sequence.

7. The rAAV vector of claim 6, wherein the second AAV ITR sequence is an AAV2 ITR sequence.

8. The rAAV vector of claim 6 or 7, wherein the second AAV ITR sequence comprises the sequence set forth in SEQ ID NO: 15

9. The rAAV vector of any one of claims 1-8, wherein the rAAV vector further comprises a promoter sequence.

10. The rAAV vector of claim 9, wherein the promoter sequence is a CBh promoter sequence.

11. The rAAV vector of claim 10, wherein the CBh promoter sequence comprises the nucleic acid sequence set forth in SEQ ID NO: 26.

12. An rAAV vector comprising, in the 5′ to 3′ direction:
(a) a first AAV2 ITR sequence;
(b) a CBh promoter sequence;
(c) a polynucleotide sequence encoding for at least one amiRNA directed against GYS1 comprising the nucleic acid sequence of any one of SEQ ID NOs: 35-39; and
(d) a second AAV2 ITR sequence.

13. The rAAV vector of claim 12, wherein the rAAV vector comprises the sequence set forth in any one of SEQ ID NOs: 6-10.

14. An rAAV viral vector comprising:
(a) an AAV capsid protein; and
(b) an rAAV vector of any one of claims 1-13.

15. The rAAV viral vector of claim 14, wherein the AAV capsid protein is an AAV9 capsid protein.

16. A pharmaceutical composition comprising the rAAV vector of any one of claims 1-13 or the rAAV viral vector of claim 14 or 15; and at least one pharmaceutically acceptable excipient and/or additive.

17. A method for treating a subject having a glycogen storage disorder, the method comprising administering to the subject a therapeutically effective amount of the rAAV vector of any one of claims 1-13, the rAAV viral vector of claim 14 or 15, or the pharmaceutical composition of claim 16.

18. The method of claim 17, wherein the rAAV vector, the rAAV viral vector, or the pharmaceutical composition is administered to the subject at a dose ranging from about 10¹¹ to about 10¹⁸ viral vector particles.

19. The method of claim 17 or 18, wherein the rAAV vector, the rAAV viral vector, or pharmaceutical composition is administered intravenously or intrathecally.

20. The method of any one of claims 17-19, wherein the glycogen storage disorder is Lafora Disease or adult polyglucosan body disease.

21. The rAAV vector of any one of claims 1-13, the rAAV viral vector of claim 14 or 15, or the pharmaceutical composition of claim 16 for use in the treatment of a glycogen storage disorder.

22. The use of claim 21, wherein the rAAV vector, the rAAV viral vector, or the pharmaceutical composition is for administration to a subject at a dose ranging from about 10¹¹ to about 10¹⁸ viral vector particles.

23. The use of claim 21 or 22, wherein the rAAV vector, the rAAV viral vector, or pharmaceutical composition is for intravenous or intrathecal administration.

24. The method of any one of claims 21-23, wherein the glycogen storage disorder is Lafora Disease or adult polyglucosan body disease.
